(19) United States
(12) Patent Application Publication
(10) Pub. No.: US 2025/0388939 A1
(43) Pub. Date: Dec. 25, 2025

(54) RECOMBINANT HOST CELLS WITH IMPROVED PRODUCTION OF L-DOPA, DOPAMINE, S-NOROCLAURINE OR DERIVATIVES THEREOF

(71) Applicant: RIVER STONE BIOTECH APS, Copenhagen (DK)

(72) Inventors: Esben Halkjaer HANSEN, Copenhagen (DK); Jens HOUGHTON-LARSEN, Copenhagen (DK)

(21) Appl. No.: 19/248,782

(22) Filed: Jun. 25, 2025

Related U.S. Application Data
(62) Division of application No. 17/421,967, filed on Jul. 9, 2021, now Pat. No. 12,371,722.

(30) Foreign Application Priority Data
Jan. 11, 2019 (DK) PA201900035

(51) Int. Cl. C12P 13/00; C12N 9/02; C12N 15/81; C12R 1/865

(52) U.S. Cl. CPC C12P 13/001; C12N 9/0071; C12N 15/81; C12R 2001/865; C12Y 114/16002

(57) ABSTRACT

The present invention relates to a recombinant microbial host cell comprising an operative biosynthetic metabolic pathway capable of producing one or more compounds selected from the group consisting of L-dopa, dopamine, (S)-Norcoclaurine and derivatives thereof; said pathway comprising a heterologous L-tyrosine hydroxylase (TyrH) converting L-Tyrosine into L-dopa capable of increasing the cell production of the Compound compared to a reference L-tyrosine hydroxylase having the sequence set forth in SEQ ID NO: 58.

### CROSS REFERENCE

This application is divisional application of U.S. application Ser. No. 17/421,967, filed Jul. 9, 2021, which is a U.S. national phase application under 35 U.S.C. § 371 of International Application No. PCT/EP2020/050610, filed Jan. 10, 2020, which claims the benefit of DK Application No. PA201900035, filed Jan. 11, 2019, the disclosures of each of which are explicitly incorporated by reference herein in their entirety.

### SEQUENCE LISTING

This application contains a sequence listing which is submitted electronically and is hereby incorporated by reference in its entirety. The sequence listing submitted herewith is contained in the XML filed created Sep. 11, 2025 entitled “21-0818-WO-US-DIV_Sequence-Listing.xml” and is 141,649 bytes in size.

### FIELD OF THE INVENTION

The present invention relates to recombinant host cells producing the compounds L-DOPA, dopamine and(S)-Norcoclaurine or derivatives thereof using tyrosine hydroxylase; to recombinant polynucleotides comprising a sequence encoding tyrosine hydroxylase, operably linked to promoter nucleotide sequences facilitating expression of the tyrosine hydroxylase. Further, the invention relates to cell cultures comprising the host cell of the invention, to methods of producing the compounds of the invention; to fermentation liquids comprising the compounds resulting from such methods, to compositions comprising the fermentation liquid; to pharmaceutical preparations made from such compositions and to the use of such compositions and preparations.

### BACKGROUND OF THE INVENTION

L-3,4-dihydroxyphenylalanine (L-DOPA) is an intermediate metabolite/precursor in the biosynthetic pathway for many compounds, including benzylisoquinoline alkaloids (BIAs), where L-DOPA is a key precursor in the formation of dopamine and in turn(S)-norcoclaurine, which is the first committed intermediate in BIA pathways. BIA are known to have diverse pharmaceutical properties including, for example, analgesic, antimicrobial, antitussive, antiparasitic, cytotoxic, and anticancer properties (Hagel & Facchini, 2013, Plant Cell Physiol. 54 (5); 647-672). Thousands of distinct BIAs have been identified in plants, each of which derive from a common precursor: (S)-norcoclaurine (see e.g., Hagel & Facchini, 2013, Plant Cell Physiol. 54 (5); 647-672; Fossati et al., 2015, PLOS ONE 10 (4): e0124459).

While it is known that production of these complex alkaloid compounds, in planta, requires plant cells to perform a plethora of different enzyme mediated chemical reactions in concert (pathways).

While it is in principle understood that plant enzyme polypeptides and polynucleotides encoding them, are instrumental for in planta synthesis of alkaloids, many aspects of alkaloid pathways are yet to be explored, not only which polypeptides are relevant for producing a particular alkaloids in nature, but also which polypeptides can be can be implemented to produce alkaloids ex planta, for example in heterologous host cells, and in particular which polypeptides are capable of producing better yields of desired alkaloids when produced by ex planta biosynthetic manufacturing methods.

L-tyrosine hydroxylases are polypeptides involved in hydroxylating L-tyrosine into L-DOPA. Galanie et al.: “Complete biosynthesis of opioids in yeast”, Science, 2015, Vol 349, No. 6252, pages 1095-1100 pertains to an engineered biosynthetic pathway producing thebaine and hydrocodone in yeast including a genetically modified mammalian tyrosine hydroxylase from Rattus norvegicus. WO 2017/122189 (Yeda Research and Development Co) discloses sequences said to encode enzymes capable of converting tyrosine into L-DOPA and methods for producing L-DOPA in a cell using such enzymes. WO 2018/005553 (Facchini et al) asserts that BIAs can be produced in cells using the tyrosine hydroxylase CYP76AD1 for converting L-tyrosine to L-DOPA. WO2016/049364 (Martin et al.) and DELOACHE, C. W. et al.; Nature Chemical Biology; 2015; Vol. 11; pages 465-471, discloses a variant or mutant of CYP76AD1 tyrosine hydroxylases (referred to herein as SEQ ID NO: 58) said to provide for increased production of L-DOPA in host cells expressing this tyrosine hydroxylase.

### SUMMARY OF THE INVENTION

The inventors of the present invention have identified L-tyrosine Hydroxylases (TyrH's), which not only surprisingly integrate and work in recombinant host cells, but also exhibit significant improvements in producing L-DOPA and subsequently dopamine, (S)-norcoclaurine or derivatives thereof in the host cell over hitherto known best TyrH's. Accordingly, in a first aspect the invention provides a recombinant microbial host cell comprising an operative biosynthetic metabolic pathway capable of producing one or more target compounds selected from the group consisting of L-dopa, dopamine, (S)-Norcoclaurine and derivatives thereof; said pathway comprising one or more heterologous L-tyrosine hydroxylases (TyrH) converting L-Tyrosine into L-dopa capable of increasing the cell production of the target compound(s) compared to a reference L-tyrosine hydroxylase having the sequence set forth in SEQ ID NO: 58.

In a further aspect the invention provides a nucleic acid construct comprising a polynucleotide sequence encoding the TyrH of the invention, operably linked to one or more control sequences heterologous to the TyrH encoding polynucleotide.

In a further aspect the invention provides an expression vector comprising the nucleic acid construct of the invention.

In a further aspect the invention provides a recombinant microbial host cell comprising the nucleic acid construct or the vector of the invention.

In a further aspect the invention provides a cell culture, comprising the host cell of the invention. and a growth medium.

In a further aspect the invention provides a method for producing at least one target compound selected from the group consisting of one or more of L-dopa, dopamine and(S)-Norcoclaurine or a derivative thereof comprising
a) culturing the cell culture of the invention at conditions allowing the host cell to produce the target compound; and
b) optionally recovering and/or isolating the target compound.

In a further aspect the invention provides a fermentation liquid comprising the at least one target compound selected from L-dopa, dopamine, (S)-Norcoclaurine and derivatives thereof comprised in the cell culture of the invention.

In a further aspect the invention provides a composition comprising the fermentation liquid of the invention and one or more agents, additives and/or excipients.

In a further aspect the invention provides a method for preparing a pharmaceutical preparation comprising subjecting a composition of the invention to one or more steps of converting the target compound in the composition to a pharmaceutically active derivative selected from the group consisting of Berberine, Papaverine, Morphine, Sanguinarine, Noscapine, Neomorphine, hydrocodone, Codeine, Oxycodone, Oxymorphone, Dihydromorphine and buprenorphine; and mixing the derivative with one or more pharmaceutical grade additives and/or adjuvants.

In a further aspect the invention provides a pharmaceutical preparation obtainable from the method of the invention for preparing the pharmaceutical preparation.

In a final aspect the invention provides a method for treating pain or opioid poisoning in a mammal comprising administering the pharmaceutical preparation of the invention to the mammal.

### DESCRIPTION OF DRAWINGS AND FIGURES

FIG. 1 depicts the Shikimate pathway to L-tyrosine and additional steps for producing(s)-norcoclaurine.

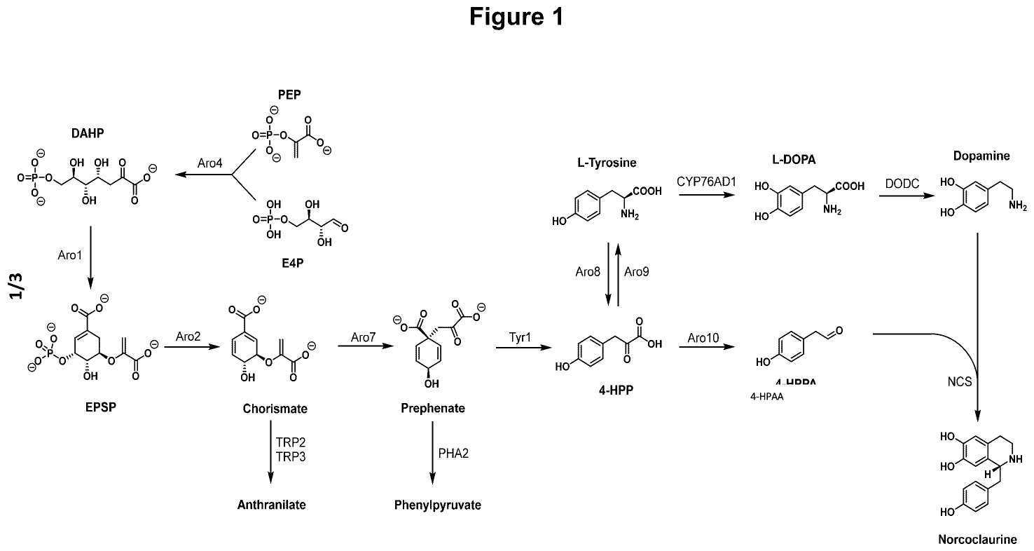

FIG. 2 depicts a range of compounds having pharmaceutical properties which are derivatives of(S)-norcoclaurine.

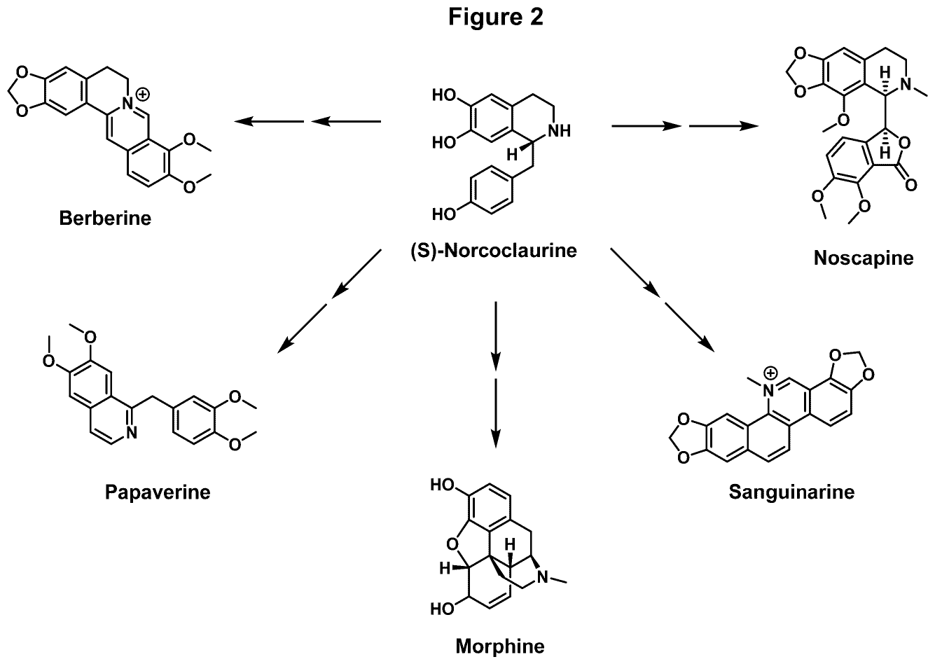

FIG. 3 depicts the pathway of steps for producing thebaine from glucose.

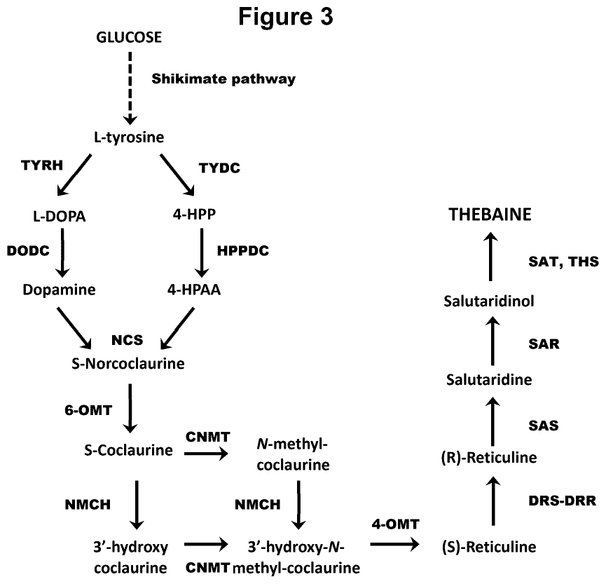

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION OF THE INVENTION

### Definitions

The term AUC as used herein refers to area under the curve, determined by the integration of the peaks representative of analytes described in Example 1.

The term “PEP” as used herein refers to phosphoenol pyruvate

The term “E4P” as used herein refers to erythrose-4-phosphate

The term “DAHP synthase” as used herein refers to an enzyme capable of DAHP synthase activity, thus having the ability to catalyze the reaction producing DAHP from PEP and E4P. Nonlimiting examples of DAHP synthases are ARO3; YDR035W; SGD:S000002442 and ARO4; YBR249C; SGD:S000000453 as disclosed in the saccharomyces genome database (SGD) and natively found in S. cerevisiae.

The term “DAHP” as used herein refers to 3-deoxy-D-arabino-2-heptulosonic acid 7-phosphate.

The term “EPSP synthase” as used herein refers to an enzyme capable of catalyzing the conversion of DAHP into EPSP. A nonlimiting example of an EPSP synthase is ARO1; YDR127W; SGD:S000002534 as disclosed in the saccharomyces genome database (SGD) and natively found in S. cerevisiae.

The term “EPSP” as used herein refers to 5-enolpyruvylshikimate-3-phosphate.

The term “chorismate synthase” as used herein refers to an enzyme capable of catalyzing the conversion of EPSP into chorismate. A nonlimiting example of a chorismite synthase is ARO2; YGL148W; SGD:S000003116 as disclosed in the saccharomyces genome database (SGD) and natively found in S. cerevisiae.

The term “prephenate dehydrogenase” as used herein refers to an enzyme capable of catalyzing the conversion of prephenate into 4-HPP. A nonlimiting example of a prephenate dehydrogenase is TYR1 (YBR166C; SGD:S000000370 as disclosed in the saccharomyces genome database (SGD) at www.yeastgenome.org) natively found in S. cerevisiae.

The term “4-HPP” as used herein refers to 4-hydroxyphenylpyruvate

The term “aromatic aminotransferase” as used herein refers to an enzyme capable of catalyzing the conversion of 4-HPP into L-tyrosine. Nonlimiting examples of aromatic aminotransferases are ARO8 and ARO9 (YGL202W; SGD:S000003170 and YHR137W; SGD:S000001179 as disclosed in the saccharomyces genome database (SGD) and natively found in S. cerevisiae.

The term “HPPDC” as used herein refers to hydroxyphenylpyruvate decarboxylase catalyzing 4-HPP into 4-HPAA. A nonlimiting example of an HPPDC is ARO10 (GenBank accession no. NP_010668.3) natively found in S. cerevisiae.

The term “4-HPAA” as used herein refers to 4-Hydroxyphenylacetaldehyde.

The term “TyrH” as used herein refers to tyrosine hydroxylase catalyzing L-tyrosine into L-DOPA.

The term “CPR” as used herein refers to P450 reductase catalyzing the electron transfer from NADPH to cytochrome P450, typically in the endoplasmic reticulum of a eukaryotic cell.

The term “Cytochrome P450 enzyme” or “P450 enzymes” or “P450” as used herein interchangeably refers to a family of monooxygenases enzymes containing heme as a cofactor. P450's are also known as “CYP's”.

The term “DODC” and TYDC” as used herein refers to L-dopa decarboxylase and tyrosine decarboxylase respectively catalyzing conversion of L-DOPA into dopamine and tyrosine into 4-HPP.

The term “MAO” as used herein refers to monoamine oxidase catalyzing conversion of dopamine to 3,4 DHPAA

The term “DHPAA” as used herein refers to 3,4-dihydroxyphenylacetaldehyde.

The term “NCS” as used herein refers to Norcoclaurine synthase catalyzing conversion of dopamine and 4-HPAA into Norcoclaurine.

The term “6-OMT” as used herein refers to 6-O-methyltransferase catalyzing conversion of(S)-norcoclaurine to(S)-Coclaurine

The term “CNMT” as used herein refers to Coclaurine-N-methyltransferase catalyzing conversion of(S)-Coclaurine to(S)-N-Methylcoclaurine and(S)-3′-hydroxycoclaurine to(S)-3′-hydroxy-N-methyl-coclaurine.

The term “NMCH” as used herein refers to N-methylcoclaurine 3′-monooxygenase catalyzing conversion of(S)-Coclaurine to(S)-3′-hydroxycoclaurine and(S)-N-Methylcoclaurine to(S)-3′-Hydroxy-N-Methylcoclaurine

The term “4′-OMT” as used herein refers to 3′-hydroxy-N-methyl-(S)-coclaurine 4′-O-methyltransferase catalyzing conversion of(S)-3′-Hydroxy-N-Methylcoclaurine to(S)-Reticuline.

The term “DRS-DRR” as used herein refers to 1,2-dehydroreticuline synthase-1,2-dehydroreticuline reductase complex catalyzing conversion of(S)-Reticuline to (R)-Reticuline.

The term “SAS” as used herein refers to salutaridine synthase catalyzing conversion of (R)-Reticuline to Salutaridine.

The term “SAR” as used herein refers to salutaridine reductase catalyzing conversion of Salutaridine to Salutaridinol.

The term “SAT” as used herein refers to salutaridinol 7-O-acetyltransferase catalyzing conversion of Salutaridinol to 7-O-acylsalutaridinol.

The term “THS” as used herein refers to thebaine synthase catalyzing conversion of 7-O-acylsalutaridinol to thebaine.

The term “BIA” or “benzylisoquinoline alkaloid” as used herein refers to a compound of the general formula:

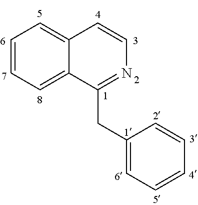

which is the structural backbone of many alkaloids with a wide variety of structures.

The term “heterologous” or “recombinant” and its grammatical equivalents as used herein refers to entities “derived from a different species or cell”. For example, a heterologous or recombinant polynucleotide gene is a gene in a host cell not naturally containing that gene, i.e. the gene is modified to a non-naturally occurring form or it is from a different species or cell type than the host cell.

The term “recombinant host cell” as used herein refers to host cell comprising and expressing heterologous or recombinant polynucleotide genes.

The term “substrate” or “precursor”, as used herein refers to any compound that can be converted into a different compound. For example, L-tyrosine can be a substrate for TyrH and can be converted into L-DOPA. For clarity, substrates and/or precursors include both compounds generated in situ by a enzymatic reaction in a cell or exogenously provided compounds, such as exogenously provided organic molecules which the host cell can metabolize into a desired compound.

The term “metabolic pathway” as used herein is intended to mean two or more enzymes acting sequentially in a live cell to convert chemical substrate(s) into chemical product(s). Enzymes are characterized by having catalytic activity, which can change the chemical structure of the substrate(s). An enzyme may have more than one substrate and produce more than one product. The enzyme may also depend on cofactors, which can be inorganic chemical compounds or organic compounds such as proteins for example enzymes (co-enzymes). The CPR that reduces the Cytochrome P450 is an example of an enzymatic co-factor. The term “operative biosynthetic metabolic pathway” refers to a metabolic pathway that occurs in a live recombinant host, as described herein.

The term “in vivo”, as used herein refers to within a living cell, including, for example, a microorganism or a plant cell.

The term “in vitro”, as used herein refers to outside a living cell, including, without limitation, for example, in a microwell plate, a tube, a flask, a beaker, a tank, a reactor and the like.

Term “endogenous” or “native” as used herein refers to a gene or a polypeptide in a host cell which originates from the same host cell.

The term “deletion” as used herein refers to manipulation of a gene so that it is no longer present or partially present, so that the gene is not expressed in a host cell.

The term “disruption” as used herein refers to the genetic manipulation of a gene or any of the machinery participating in the expression the gene, so that it is no longer expressed in a host cell. Non-limiting examples of methods of genetic disruption include nonsense mutations, knockouts, knockins, antisense silencing, and so on.

The term “attenuation” or “downregulation” as used herein refers to manipulation of a gene or any of the machinery participating in the expression the gene, so that it the expression of the gene is reduced as compared to expression without the manipulation.

The terms “substantially” or “approximately” or “about”, as used herein refers to a reasonable deviation around a value or parameter such that the value or parameter is not significantly changed. These terms of deviation from a value should be construed as including a deviation of the value where the deviation would not negate the meaning of the value deviated from. For example, in relation to a reference numerical value the terms of degree can include a range of values plus or minus 10% from that value. For example, using these deviating terms can also include a range deviations plus or minus such as plus or minus 9%, 8%, 7%, 6%, 5%, 4%, 3%, 2%, or 1% from a specified value.

The term “and/or” as used herein is intended to represent an inclusive “or”. The wording X and/or Y is meant to mean both X or Y and X and Y. Further the wording X, Y and/or Z is intended to mean X, Y and Z alone or any combination of X, Y, and Z.

The term “isolated” as used herein about a compound, refers to any compound, which by means of human intervention, has been put in a form or environment that differs from the form or environment in which it is found in nature. Isolated compounds include but is no limited to compounds of the invention for which the ratio of the compounds relative to other constituents with which they are associated in nature is increased or decreased. In an important embodiment the amount of compound is increased relative to other constituents with which the compound is associated in nature.

In an embodiment the compound of the invention may be isolated into a pure or substantially pure form. In this context a substantially pure compound means that the compound is separated from other extraneous or unwanted material present from the onset of producing the compound or generated in the manufacturing process. Such a substantially pure compound preparation contains less than 10%, such as less than 8%, such as less than 6%, such as less than 5%, such as less than 4%, such as less than 3%, such as less than 2%, such as less than 1%, such as less than 0.5% by weight of other extraneous or unwanted material usually associated with the compound when expressed natively or recombinantly. In an embodiment the isolated compound is at least 90% pure, such as at least 91% pure, such as at least 92% pure, such as at least 93% pure, such as at least 94% pure, such as at least 95% pure, such as at least 96% pure, such as at least 97% pure, such as at least 98% pure, such as at least 99% pure, such as at least 99.5% pure, such as 100% pure by weight.

The term “non-naturally occurring” as used herein about a substance, refers to any substance that is not normally found in nature or natural biological systems. In this context the term “found in nature or in natural biological systems” does not include the finding of a substance in nature resulting from releasing the substance to nature by deliberate or accidental human intervention. Non-naturally occurring substances may include substances completely or partially synthetized by human intervention and/or substances prepared by human modification of a natural substance.

The term “% identity” is used herein about the relatedness between two amino acid sequences or between two nucleotide sequences.

The term “% identity” as used herein about amino acid sequences refers to the degree of identity in percent between two amino acid sequences obtained when using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

$$\frac{identical amino acids residues}{Length of alignment - total number of gaps in alignment} \times 100$$

The term “% identity” as used herein about nucleotide sequences refers to the degree of identity in percent between two nucleotide sequences obtained when using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

$$\frac{identical deoxyribonucleotides}{Length of alignment - total number of gaps in alignment} \times 100$$

The protein sequences of the present invention can further be used as a “query sequence” to perform a search against sequence databases, for example to identify other family members or related sequences. Such searches can be performed using the BLAST programs. Software for performing BLAST analyses is publicly available through the National Center for Biotechnology Information.

BLASTP is used for amino acid sequences and BLASTN for nucleotide sequences. The BLAST program uses as defaults:
Cost to open gap: default=5 for nucleotides/11 for proteins
Cost to extend gap: default=2 for nucleotides/1 for proteins
Penalty for nucleotide mismatch: default=−3
Reward for nucleotide match: default=1
Expect value: default=10
Wordsize: default=11 for nucleotides/28 for megablast/3 for proteins

Furthermore, the degree of local identity between the amino acid sequence query or nucleic acid sequence query and the retrieved homologous sequences is determined by the BLAST program. However only those sequence segments are compared that give a match above a certain threshold. Accordingly, the program calculates the identity only for these matching segments. Therefore, the identity calculated in this way is referred to as local identity.

The term “cDNA” refers to a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

The term “coding sequence” refers to a nucleotide sequence, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The term “control sequence” as used herein refers to a nucleotide sequence necessary for expression of a polynucleotide encoding a polypeptide. A control sequence may be native (i.e., from the same gene) or heterologous or foreign (i.e., from a different gene) to the polynucleotide encoding the polypeptide. Control sequences include, but are not limited to leader sequences, polyadenylation sequence, pro-peptide coding sequence, promoter sequences, signal peptide coding sequence, translation terminator (stop) sequences and transcription terminator (stop) sequences. To be operational control sequences usually must include promoter sequences, transcriptional and translational stop signals. Control sequences may be provided with linkers for the purpose of introducing specific restriction sites facilitating ligation of the control sequences with a coding region of a polynucleotide encoding a polypeptide.

The term “expression” includes any step involved in the production of a polypeptide including, but not limited to, transcription, post-transcriptional modification, translation, post-translational modification, and secretion.

The term “expression vector” refers to a DNA molecule, either single- or double stranded, either linear or circular, which comprises a polynucleotide encoding a polypeptide and is operably linked to control sequences that provide for its expression. Expression vectors include expression cassettes for the integration of genes into a host cell as well as plasmids and/or chromosomes comprising such genes.

The term “host cell” refers to any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

The term “nucleic acid construct” refers to a nucleic acid molecule, either single- or double stranded, which is isolated from a naturally occurring gene or is modified to contain segments of nucleic acids in a manner that would not otherwise exist in nature or which is synthetic, and which comprises one or more control sequences.

The term “operably linked” refers to a configuration in which a control sequence is placed at an appropriate position relative to the coding polynucleotide such that the control sequence directs expression of the coding polynucleotide.

The terms “nucleotide sequence and “polynucleotide” are used herein interchangeably.

The term “comprise” and “include” as used throughout the specification and the accompanying claims as well as variations such as “comprises”, “comprising”, “includes” and “including” are to be interpreted inclusively. These words are intended to convey the possible inclusion of other elements or integers not specifically recited, where the context allows.

The articles “a” and “an” are used herein refers to one or to more than one (i.e. to one or at least one) of the grammatical object of the article. By way of example, “an element” may mean one element or more than one element.

Terms like “preferably”, “commonly”, “particularly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

The term “cell culture” as used herein refers to a culture medium comprising a plurality of recombinant host cells of the invention. A cell culture may comprise a single strain of recombinant host or may comprise two or more distinct host strains. The culture medium may be any medium that may comprise a recombinant host, e.g., a liquid medium (i.e., a culture broth) or a semi-solid medium, and may comprise additional components, e.g., a carbon source such as dextrose, sucrose, glycerol, or acetate; a nitrogen source such as ammonium sulfate, urea, or amino acids; a phosphate source; vitamins; trace elements; salts; amino acids; nucleobases; yeast extract; aminoglycoside antibiotics such as G418 and hygromycin B.

### Recombinant Host Cells

The invention provides the first aspect recombinant microbial host cell comprising an operative biosynthetic metabolic pathway capable of producing one or more target compounds selected from the group consisting of L-dopa, dopamine, (S)-Norcoclaurine and derivatives thereof; said pathway comprising one or more heterologous L-tyrosine hydroxylases (TyrH) converting L-Tyrosine into L-dopa capable of increasing the cell production of the target compound(s) compared to a host cell using the hitherto best known reference TyrH having the sequence set forth in SEQ ID NO: 58. In a particular embodiment the host cell increases production of the target compound(s) by at least 50%, such as at least 100%, such as least 150%, such as at least 200%. In particular the inventors have found a group of TyrH which performs particularly well and in an embodiment the one or more TyrH of the invention has at least 70% identity to a polypeptide selected from the group consisting of SEQ ID NO: 2; to SEQ ID NO: 4; to SEQ ID NO: 10; to SEQ ID NO: 6; to SEQ ID NO: 24; to SEQ ID NO: 8; to SEQ ID NO: 12; to SEQ ID NO: 14; and/or to SEQ ID NO: 16. In a more specific embodiment the TyrH has at least 75%, such as at least 80%, such as at least 90%, such as at least 95%, such as at least 99%, such as at least 100% identity to a polypeptide selected from the group consisting of SEQ ID NO: 2; to SEQ ID NO: 4; to SEQ ID NO: 10; to SEQ ID NO: 6; to SEQ ID NO: 24; to SEQ ID NO: 8; to SEQ ID NO: 12; to SEQ ID NO: 14; and/or to SEQ ID NO: 16.

In an embodiment, the operative biosynthetic metabolic pathway in the host cell of the invention further comprises and expresses one or more genes encoding additional pathway enzyme polypeptides selected from the group consisting of:
a) DAHP synthase;
b) EPSP synthase
c) chorismate synthase;
d) chorismate mutase;
e) prephenate dehydrogenase;
f) aromatic aminotransferase;
g) CPR;
h) DODC;
i) TYDC;
j) HPPDC;
k) MAO;
l) NCS;
m) 6-OMT;
n) CNMT;
o) NMCH;
p) 4′-OMT;
q) DRS-DRR;
r) SAS;
s) SAR;
t) SAT and
u) THS.

In an embodiment the host cell comprise all enzyme polypeptides required to produce a desired compound from simple nutrient substrates such as glucose fed from a fermentation medium. However, since substrates and precursors may be provided to the host cell exogenously, the host cell pathway may comprise any combination of selected pathway enzyme polypeptides, depending on the exogenously provided precursor and the compound desired to be produced by the host cell.

In an embodiment the operative pathway in the host cell comprises DAHP synthase; EPSP synthase chorismate synthase; chorismate mutase; prephenate dehydrogenase; aromatic aminotransferase; CPR; DODC; TYDC; HPPDC; and NCS. More specifically the chorismate mutase; CPR; DODC; TYDC; and NCS may all be heterologous to the host cell.

In a further embodiment the operative pathway in the host cell comprises DAHP synthase; EPSP synthase chorismate synthase; chorismate mutase; prephenate dehydrogenase; aromatic aminotransferase; CPR; DODC; TYDC; HPPDC; NCS; 6-OMT; CNMT; NMCH; 4′-OMT; DRS-DRR; SAS; SAR;

SAT; and optionally THS. Conversion of 7-O-acylsalutaridinol into thebaine may occur to a certain extent spontaneously, but the rate can be significantly increased by inclusion of THS.

In an embodiment the corresponding:
a) DAHP synthase is a native yeast DAHP synthase, such as the ARO3; YDR035W; SGD:S000002442 or ARO4; YBR249C; SGD:S000000453 as disclosed in the saccharomyces genome database (SGD);
b) EPSP synthase is a native yeast EPSP synthase such as the ARO1; YDR127W; SGD:S000002534 as disclosed in the saccharomyces genome database (SGD);
c) chorismate synthase is a native yeast chorismate synthase such as the ARO2; YGL148W; SGD:S000003116 as disclosed in the saccharomyces genome database (SGD);
d) chorismate mutase is a native yeast chorismate mutase and/or chorismate mutase which has at least 70%, such as at least 75%, such as at least 80%, such as at least 90%, such as at least 95%, such as at least 99%, such as at least 100% identity to the chorismate synthase of SEQ ID NO: 77, or alternatively the ARO7; YPRO60C; SGD:S000006264 as disclosed in the saccharomyces genome database (SGD);
e) prephenate dehydrogenase is a native yeast prephenate dehydrogenase such as the TYR1; YBR166C; SGD:S000000370 as disclosed in the saccharomyces genome database (SGD);
f) aromatic aminotransferase is a native yeast aromatic aminotransferase such as the ARO8; YGL202W; SGD:S000003170 or ARO9; YHR137W; SGD:S000001179 as disclosed in the saccharomyces genome database (SGD);
g) CPR has at least 70%, such as at least 75%, such as at least 80%, such as at least 90%, such as at least 95%, such as at least 99%, such as at least 100% identity to the CPR of SEQ ID NO: 76;
h) DODC has at least 70%, such as at least 75%, such as at least 80%, such as at least 90%, such as at least 95%, such as at least 99%, such as at least 100% identity to the DODC of SEQ ID NO: 60; and/or is encoded by the gene disclosed in GenBank accession no. AE015451.
i) TYDC is encoded by the gene disclosed in GenBank accession nos. P54768 (Papaver somniferum); GenBank accession nos. U08597 (Papaver somniferum); or GenBank accession no. AF314150 (Thalictrum flavum);
j) HPPDC is encoded by the gene disclosed in GenBank accession no. NP_010668.3 (S. cerevisiae);
k) MAO is encoded by the gene disclosed in GenBank accession no. AB010716 (Micrococcus luteus);
l) NCS has at least 70%, such as at least 75%, such as at least 80%, such as at least 90%, such as at least 95%, such as at least 99%, such as at least 100% identity to the NCS of SEQ ID NO: 61 or SEQ ID NO: 75; and/or is the NCS of SEQ ID NO: 24 disclosed in WO2018/029282 (S. cerevisiae codon optimised) or the NCS's disclosed in DK patent application PA 2017 70533 or is encoded by the gene disclosed in GenBank accession no. AB267399.2 (Coptis japonica):
m) 6-OMT has at least 70%, such as at least 75%, such as at least 80%, such as at least 90%, such as at least 95%, such as at least 99%, such as at least 100% identity to the 6-OMY of SEQ ID NO: 62; or is the 6-OMT encoded by the gene disclosed in GenBank accession no. Q6WUC1 (Papaver somniferum) or GenBank accession no. D29811 (Coptis japonica);
n) CNMT has at least 70%, such as at least 75%, such as at least 80%, such as at least 90%, such as at least 95%, such as at least 99%, such as at least 100% identity to the CNMT of SEQ ID NO: 63; or is the CNMT encoded by the gene disclosed in GenBank accession no. Q948P7 (Coptis japonica) or GenBank accession no. AY610508 (Thalictrum flavum) or GenBank accession no. AY217336 (Papaver somniferum);
o) NMCH has at least 70%, such as at least 75%, such as at least 80%, such as at least 90%, such as at least 95%, such as at least 99%, such as at least 100% identity to the NMCH of SEQ ID NO: 65; or is the NMCH encoded by the gene disclosed in GenBank accession no. 064899 (Papaver somniferum);
p) 4′-OMT has at least 70%, such as at least 75%, such as at least 80%, such as at least 90%, such as at least 95%, such as at least 99%, such as at least 100% identity to the 4′-OMT of SEQ ID NO: 66; or is the 4′-OMT encoded by the gene disclosed in GenBank accession no. Q9LEL5 (Coptis japonica);
q) DRS-DRR has at least 70%, such as at least 75%, such as at least 80%, such as at least 90%, such as at least 95%, such as at least 99%, such as at least 100% identity to the DRS-DRR of SEQ ID NO: 68; or is the DRS-DRR encoded by the gene disclosed in GenBank accession no. PODKI7 (Papaver somniferum) or the DRS-DRR disclosed in Smolke et al.; Science. 2015 Sep. 4; 349 (6252): 1095-1100;
r) SAS has at least 70%, such as at least 75%, such as at least 80%, such as at least 90%, such as at least 95%, such as at least 99%, such as at least 100% identity to the SAS of SEQ ID NO: 70; or is the SAS encoded by the gene disclosed in GenBank accession no. EF451150 (Papaver somniferum);
s) SAR has at least 70%, such as at least 75%, such as at least 80%, such as at least 90%, such as at least 95%, such as at least 99%, such as at least 100% identity to the SAR of SEQ ID NO: 71; or is the SAR encoded by the gene disclosed in GenBank accession no. DQ316261 (Papaver somniferum)
t) SAT has at least 70%, such as at least 75%, such as at least 80%, such as at least 90%, such as at least 95%, such as at least 99%, such as at least 100% identity to the SAT of SEQ ID NO: 73; or is the SAT encoded by the gene disclosed in GenBank accession no. AF339913 (Papaver somniferum); and
u) THS has at least 70%, such as at least 75%, such as at least 80%, such as at least 90%, such as at least 95%, such as at least 99%, such as at least 100% identity to the THS of SEQ ID NO: 79 or SEQ ID NO: 80.

In a particular embodiment the the operative biosynthetic metabolic pathway of the invention comprises:
a) chorismate mutase having at least 95% identity to the chorismate synthase of SEQ ID NO: 77;
b) CPR having at least 95% to the CPR of SEQ ID NO: 76;
c) DODC having at least 95% to the DODC of SEQ ID NO: 60;
d) TyrH having at least 95% to the TyrH of SEQ ID NO: 2;
e) NCS having at least 95% to the NCS of SEQ ID NO: 61 or SEQ ID NO: 75;
f) 6-OMT having at least 95% to the 6-OMT of SEQ ID NO: 62;
g) CNMT having at least 95% to the CNMT of SEQ ID NO: 63;
h) NMCH having at least 95% to the NMCH of SEQ ID NO: 65;
i) 4′-OMT having at least 95% to the 4′-OMT of SEQ ID NO: 66;
j) DRS-DRR having at least 95% to the DRS-DRR of SEQ ID NO: 68;
k) SAS which has at least 95% to the SAS of SEQ ID NO: 70;
l) SAR which has at least 95% to the SAR of SEQ ID NO: 71;
m) SAT having at least 95% to the SAT of SEQ ID NO: 73; and
n) THS having at least 95% to the THS of SEQ ID NO: 79 or SEQ ID NO: 80.

The recombinant host cell of the invention is capable of producing one or more target compounds selected from L-dopa, dopamine and(S)-Norcoclaurine or derivatives thereof. In an embodiment the derivatives of L-dopa, dopamine and(S)-Norcoclaurine is a benzylisoquinoline alkaloid (BIA) and more specifically the BIA may selected from one or more of(S)-Norcoclaurine; (S)-Norlaudanosoline; (S)-Coclaurine; (S)-3′-Hydroxy-coclaurine; (S)-N-Methylcoclaurine; (S)-3′-Hydroxy-N-Methylcoclaurine; (S)-Reticuline; (R)-Reticuline; Salutaridine; Salutaridinol; and Thebaine. In particular the BIA is Thebaine.

One or more enzyme polypeptides of the operative biosynthetic metabolic pathway of invention are heterologous to the recombinant host cell host and particularly a plurality of enzyme polypeptides are heterologous such as 2, 3, 4, 5, 6, 7, 8, 9, 10, 11, 12, 13, 14, 15, 16, 17, 17, 18, 19 or 20 of the pathway enzyme polypeptides are heterologous to the host cell.

The host cell is in one embodiment a eukaryotic cell selected from the group consisting of mammalian, insect, plant, or fungal cells. The host cell may be a fungal cell selected from phylas consisting of Ascomycota, Basidiomycota, Neocallimastigomycota, Glomeromycota, Blastocladiomycota, Chytridiomycota, Zygomycota, Oomycota and Microsporidia. In particular the host cell is a yeast cell selected from the group consisting of ascosporogenous yeast (Endomycetales), basidiosporogenous yeast, and Fungi Imperfecti yeast (Blastomycetes), particularly a yeast cell is selected from the genera consisting of Saccharomyces, Kluveromyces, Candida, Pichia, Debaromyces, Hansenula, Yarrowia, Zygosaccharomyces, and Schizosaccharomyces.For specific species the yeast host cell may be selected from the species consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica. In another embodiment the host cell is filamentous fungus. Suitable filamentous fungal host cell may be selected among the phylas consisting of Ascomycota, Eumycota and Oomycota, particularly selected from the genera of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma. More specially a filamentous fungal host cell may be selected among the species of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporiuminops, Chrysosporiumkeratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride.

The host cell of the invention may also be further genetically modified to provide an increased amount of substrate for at least one enzyme polypeptide of the operative biosynthetic metabolic pathway and/or the host cell may be further genetically modified to exhibit increased tolerance towards one or more substrates, intermediates, or product molecules from enzyme polypeptides of the operative biosynthetic metabolic pathway.

In the alternative the host cell may be a plant cell for example of the genus Physcomitrella. In addition to plant cells the invention also provides an isolated plant, e.g., a transgenic plant, plant part comprising the pathway and TyrH of the invention and producing the compounds of the invention in useful quantities. The compound may be recovered from the plant or plant part. The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn). Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana. Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilization of the invention are also considered plant parts, e.g., embryos, endosperms, aleurone and seed coats. Also included within the scope of the present invention are the progeny of such plants, plant parts, and plant cells. The transgenic plant or plant cells comprising the operative pathway of the invention and produce the compound of the invention may be constructed in accordance with methods known in the art. In short, the plant or plant cell is constructed by incorporating one or more expression vectors of the invention into the plant host genome or chloroplast genome and propagating the resulting modified plant or plant cell into a transgenic plant or plant cell. The expression vector conveniently comprises the nucleic acid construct of the invention. The choice of regulatory sequences, such as promoter and terminator sequences and optionally signal or transit sequences, is determined, for example, on the basis of when, where, and how the pathway polypeptides is desired to be expressed. For instance, the expression of a gene encoding a pathway enzyme polypeptide may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86:506. For constitutive expression, the 358-CaMV, the maize ubiquitin 1, or the rice actin 1 promoter may be used (Franck et al., 1980, Cell 21:285-294; Christensen et al., 1992, Plant Mol. Biol. 18:675-689; Zhang et al., 1991, Plant Cell 3:1155-1165). Organ-specific promoters may be, for example, a promoter from storage sink tissues such as seeds, potato tubers, and fruits (Edwards and Coruzzi, 1990, Ann. Rev. Genet. 24:275-303), or from metabolic sink tissues such as meristems (Ito et al., 1994, Plant Mol. Biol. 24:863-878), a seed specific promoter such as the glutelin, prolamin, globulin, or albumin promoter from rice (Wu et al., 1998, Plant Cell Physiol. 39:885-889), a Vicia faba promoter from the legumin B4 and the unknown seed protein gene from Vicia faba (Conrad et al., 1998, J. Plant Physiol. 152:708-711), a promoter from a seed oil body protein (Chen et al., 1998, Plant Cell Physiol. 39:935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiol. 102:991-1000), the chlorella virus adenine methyltransferase gene promoter (Mitra and Higgins, 1994, Plant Mol. Biol. 26:85-93), the aldP gene promoter from rice (Kagaya et al., 1995, Mol. Gen. Genet. 248:668-674), or a wound inducible promoter such as the potato pin2 promoter (Xu et al., 1993, Plant Mol. Biol. 22:573-588). Likewise, the promoter may be induced by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals. A promoter enhancer element may also be used to achieve higher expression in the plant. For instance, the promoter enhancer element may be an intron that is placed between the promoter and the polynucleotide encoding a polypeptide or domain. For instance, Xu et al., 1993, supra, disclose the use of the first intron of the rice actin 1 gene to enhance expression. The selectable marker gene and any other parts of the expression construct may be chosen from those available in the art. The nucleic acid construct or expression vector is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244:1293; Potrykus, 1990, Bio/Technology 8:535; Shimamoto et al., 1989, Nature 338:274). Agrobacterium tumefaciens-mediated gene transfer is a method for generating transgenic dicots (for a review, see Hooykas and Schilperoort, 1992, Plant Mol. Biol. 19:15-38) and for transforming monocots, although other transformation methods may be used for these plants. A method for generating transgenic monocots is particle bombardment (microscopic gold or tungsten particles coated with the transforming DNA) of embryonic calli or developing embryos (Christou, 1992, Plant J. 2:275-281; Shimamoto, 1994, Curr. Opin. Biotechnol. 5:158-162; Vasil et al., 1992, Bio/Technology 10:667-674). An alternative method for transformation of monocots is based on protoplast transformation as described by Omirulleh et al., 1993, Plant Mol. Biol. 21:415-428. Additional transformation methods include those described in U.S. Pat. Nos. 6,395,966 and 7, 151,204 (both incorporated herein by reference in their entirety). Following transformation, the transformants having incorporated the expression vector or nucleic acid construct of the invention are selected and regenerated into whole plants according to methods well known in the art. Often the transformation procedure is designed for the selective elimination of selection genes either during regeneration or in the following generations by using, for example, co-transformation with two separate T-DNA constructs or site specific excision of the selection gene by a specific recombinase. In addition to direct transformation of a particular plant genotype with a nucleic acid construct of the invention, transgenic plants may be made by crossing a plant comprising the construct to a second plant lacking the construct. For example, a nucleic acid construct encoding a TyrH of the invention can be introduced into a particular plant variety by crossing, without the need for ever directly transforming a plant of that given variety. Therefore, the invention encompasses not only a plant directly regenerated from cells which have been transformed in accordance with the invention, but also the progeny of such plants. As used herein, progeny may refer to the offspring of any generation of a parent plant prepared in accordance with the present invention. Such progeny may include a nucleic acid construct of the invention. Crossing results in the introduction of a transgene into a plant line by cross pollinating a starting line with a donor plant line. Non-limiting examples of such steps are described in U.S. Pat. No. 7,151,204. Plants may be generated through a process of backcross conversion. For example, plants include plants referred to as a backcross converted genotype, line, inbred, or hybrid. Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

### Nucleotide Constructs

The invention also provides a nucleic acid construct comprising a polynucleotide sequence encoding the TyrH of the invention, operably linked to one or more control sequences heterologous to the TyrH encoding polynucleotide.

Polynucleotides may be manipulated in a variety of ways allow expression of the TyrH. Manipulation of the polynucleotide prior to its insertion into an expression vector may be desirable or necessary depending on the expression vector. The techniques for modifying polynucleotides utilizing recombinant DNA methods are well known in the art.

The control sequence may be a promoter, which is a polynucleotide that is recognized by a host cell for expression of a polynucleotide. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell. The promoter may be an inducible promoter.

Examples of suitable promoters for directing transcription of the nucleic acid construct of the invention in a filamentous fungal host cell are promoters obtained from the genes for Aspergillus nidulans acetamidase, Aspergillus niger neutral a-amylase, Aspergillus niger acid stable a-amylase, Aspergillus niger or Aspergillus awamori glucoamylase (glaA), Aspergillus gpdA promoter, Aspergillus oryzae TAKA amylase, Aspergillus oryzae alkaline protease, Aspergillus oryzae triose phosphate isomerase, A. niger or A. awamori endoxylanase (xInA) or B-xylosidase (xInD), Fusarium oxysporum trypsin-like protease (WO 96/00787), Fusarium venenatum amyloglucosidase (WO2000/56900), Fusarium venenatum Dania (WO200056900), Fusarium venenatum Quinn (WO200056900), Rhizomucor miehei lipase, Rhizomucor miehei aspartic proteinase, Trichoderma reesei β-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei β-xylosidase, as well as the NA2-tpi promoter and mutant, truncated, and hybrid promoters thereof. NA2-tpi promoter is a modified promoter from an Aspergillus neutral α-amylase gene in which the untranslated leader has been replaced by an untranslated leader from an Aspergillus triose phosphate isomerase gene. Examples of such promoters include modified promoters from an Aspergillus niger neutral α-amylase gene in which the untranslated leader has been replaced by an untranslated leader from an Aspergillus nidulans or Aspergillus oryzae triose phosphate isomerase gene. Other examples of promoters are the promoters described in WO2006/092396, WO2005/100573 and WO2008/098933, incorporated herein by reference.

Examples of suitable promoters for directing transcription of the nucleic acid construct of the invention in a yeast host include the glyceraldehyde-3-phosphate dehydrogenase promoter, PgpdA or promoters obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8:423-488. Selecting a suitable promoter for expression in yeast is well know and is well understood by persons skilled in the art.

The control sequence may also be a transcription terminator, which is recognized by a host cell to terminate transcription. The terminator is operably linked to the 3′-terminus of the polynucleotide encoding the polypeptide. Any terminator that is functional in the host cell may be used.

Useful terminators for filamentous fungal host cells are obtained from the genes for Aspergillus nidulans anthranilate synthase, Aspergillus niger glucoamylase, Aspergillus niger a-glucosidase, Aspergillus oryzae TAKA amylase, and Fusarium oxysporum trypsin-like protease.

Useful terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae a-factor, and Saccharomyces cerevisiae alcohol dehydrogenase/glyceraldehyde-3-phosphate dehydrogenase (ADH2/GAP).

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Useful polyadenylation sequences for filamentous fungal host cells are obtained from the genes for Aspergillus nidulans anthranilate synthase, Aspergillus niger glucoamylase, Aspergillus niger a-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15:5983-5990.

It may also be desirable to add regulatory sequences that regulate expression of the polypeptide relative to the growth of the host cell. Examples of regulatory systems are those that cause expression of the gene to be turned on or off in response to a chemical or physical stimulus, including the presence of a regulatory compound.

In filamentous fungi, the Aspergillus niger glucoamylase promoter, Aspergillus oryzae TAKA a-amylase promoter, and Aspergillus oryzae glucoamylase promoter may be used.

In yeast, the ADH2 system or GAL 1 system may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals.

In a particular embodiment the TyrH encoding polynucleotide in the nucleic acid construct of the invention is selected from the group of:
a) a polynucleotide having at least 70%, such at least 75%, such as at least 80%, such as at least 90%, such as at least 95%, such as at least 99%, such as at least 100% identity to SEQ ID NO: 1;
b) a polynucleotide having at least 70%, such at least 75%, such as at least 80%, such as at least 90%, such as at least 95%, such as at least 99%, such as at least 100% identity to SEQ ID NO: 3;
c) a polynucleotide having at least 70%, such at least 75%, such as at least 80%, such as at least 90%, such as at least 95%, such as at least 99%, such as at least 100% identity to SEQ ID NO: 9;
d) a polynucleotide having at least 70%, such at least 75%, such as at least 80%, such as at least 90%, such as at least 95%, such as at least 99%, such as at least 100% identity to SEQ ID NO: 5;
e) a polynucleotide having at least 70%, such at least 75%, such as at least 80%, such as at least 90%, such as at least 95%, such as at least 99%, such as at least 100% identity to SEQ ID NO: 23;
f) a polynucleotide having at least 70%, such at least 75%, such as at least 80%, such as at least 90%, such as at least 95%, such as at least 99%, such as at least 100% identity to SEQ ID NO: 7;
g) a polynucleotide having at least 70%, such at least 75%, such as at least 80%, such as at least 90%, such as at least 95%, such as at least 99%, such as at least 100% identity to SEQ ID NO: 11;
h) a polynucleotide having at least 70%, such at least 75%, such as at least 80%, such as at least 90%, such as at least 95%, such as at least 99%, such as at least 100% identity to SEQ ID NO: 13; and
i) a polynucleotide having at least 70%, such at least 75%, such as at least 80%, such as at least 90%, such as at least 95%, such as at least 99%, such as at least 100% identity to SEQ ID NO: 15.

### Expression Vectors

The invention also provides an expression vector comprising the nucleic acid construct of the invention. Various nucleotide sequences in addition to the nucleic acid construct of the invention may be joined together to produce a recombinant expression vector, which may include one or more convenient restriction sites to allow for insertion or substitution of the polynucleotide sequence encoding the TyrH of the invention at such sites. The recombinant expression vector may be any vector (e.g., a plasmid or virus) that can be conveniently subjected to recombinant DNA procedures and can bring about expression of the TyrH encoding polynucleotide. The choice of the vector will typically depend on the compatibility of the vector with the host cell into which the vector is to be introduced. The vector may be a linear or closed circular plasmid. The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may, when introduced into the host cell, integrate into the genome and replicate together with the chromosome(s) into which it has been integrated. Furthermore, a single vector or plasmid or two or more vectors or plasmids that together contain the total DNA to be introduced into the genome of the host cell, or a transposon, may be used. The vector may contain one or more selectable markers that permit easy selection of transformed, transfected, transduced, or the like cells. A selectable marker is a gene from which the product provides for biocide or viral resistance, resistance to heavy metals, prototrophy to auxotrophs, and the like.

Useful selectable markers for filamentous fungal host cell include amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Aspergillus nidulans or Aspergillus oryzae amdS and pyrG genes and a Streptomyces hygroscopicus bar gene are particularly useful in Aspergillus cells.

Useful selectable markers for yeast host cells include, but are not limited to, ADE2, HIS3, LEU2, LYS2, MET3, TRP1, and URA3.

The vector preferably contains element(s) that permits integration of the vector into the host cell's genome or permits autonomous replication of the vector in the cell independent of the genome.

For integration into the host cell genome, the vector may rely on the polynucleotide encoding the polypeptide or any other element of the vector for integration into the genome by homologous or non-homologous recombination. Alternatively, the vector may contain additional polynucleotides for directing integration by homologous recombination into the genome of the host cell at precise location(s) in the chromosome(s). To increase the likelihood of integration at a precise location, the integrational elements should contain a sufficient number of nucleic acids, such as 100 to 10,000 base pairs, such as 400 to 10,000 base pairs, and such as 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” refers to a polynucleotide that enables a plasmid or vector to replicate in vivo.

Useful origins of replication for filamentous fungal cell include AMA 1 and ANS1 (Gems et al., 1991, Gene 98:61-67; Cullen et al., 1987, Nucleic Acids Res. 15:9163-9175; WO 00/24883). Isolation of the AMA 1 gene and construction of plasmids or vectors comprising the gene can be accomplished using the methods disclosed in WO 00/24883.

Useful origins of replication for yeast host cell are the 2 micron origin of replication, ARS1, ARS4, the combination of ARS1 and CEN3, and the combination of ARS4 and CEN6.

More than one copy of a polynucleotide encoding the TyrH or other pathway enzyme polypeptides of the invention may be inserted into a host cell to increase production of an enzyme's polypeptide. An increase in the copy number can be obtained by integrating one or more additional copies of the enzyme coding sequence into the host cell genome or by including an amplifiable selectable marker gene with the polynucleotide, so that cells containing amplified copies of the selectable marker gene—and thereby additional copies of the polynucleotide—can be selected by cultivating the cells in the presence of the appropriate selectable agent. The procedures used to ligate the elements described above to construct the recombinant expression vectors of the present invention are well known to one skilled in the art (see, e.g., Sambrook et al., 1989, supra).

Accordingly, the invention also provides a recombinant host cell comprising the nucleic acid construct or the expression vector of the invention. In particular host cell comprise multiple copies of the TyrH coding polynucleotide sequence and/or of polynucleotide sequences encoding one or more pathway enzyme polypeptides of the invention. Moreover, one or more native genes of the host cell of the invention can be attenuated, disrupted and/or deleted. In one embodiment the host cell is a S. cerevisiae strain modified to delete the native gene ARI1; YGL157W; SGD:S000003125 as disclosed in the saccharomyces genome database (SGD).

### Cultures

The invention also provides a cell culture, comprising the host cell of the invention. and a growth medium. Suitable growth medium for host cells such as plant cell lines, filamentous fungi and/or yeast are known in the art.

### Methods of Producing Compounds of the Invention.

The invention also provides a method for producing at least one target compound selected from the group consisting of one or more of L-dopa, dopamine and(S)-Norcoclaurine or a derivative thereof comprising
a) culturing the cell culture of the invention at conditions allowing the host cell to produce the target compound; and
b) optionally recovering and/or isolating the target compound.

The cell culture is cultivated in a nutrient medium suitable for production of the compound of the invention and/or propagating cell count using methods known in the art. For example, the culture may be cultivated by shake flask cultivation, or small-scale or large-scale fermentation (including continuous, batch, fed-batch, or solid-state fermentations) in laboratory or industrial fermenters in a suitable medium and under conditions allowing the pathway to operate to produce the compound of the invention and optionally to be recovered and/or isolated.

The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). The selection of the appropriate medium may be based on the choice of host cell and/or based on the regulatory requirements for the host cell. Such media are in the art. The medium may, if desired, contain additional components favoring the transformed expression hosts over other potentially contaminating microorganisms. Accordingly, in an embodiment a suitable nutrient medium comprise a carbon source (e.g. glucose, maltose, molasses, starch, cellulose, xylan, pectin, lignocellolytic biomass hydrolysate, etc.), a nitrogen source (e. g. ammonium sulphate, ammonium nitrate, ammonium chloride, etc.), an organic nitrogen source (e.g. yeast extract, malt extract, peptone, etc.) and inorganic nutrient sources (e.g. phosphate, magnesium, potassium, zinc, iron, etc.).

The cultivating of the host cell may be performed over a period of from about 0.5 to about 30 days. The cultivation process may be a batch process, continuous or fed-batch process, suitably performed at a temperature in the range of 0-100° C. or 0-80° C., for example, from about 0° C. to about 50° C. and/or at a pH, for example, from about 2 to about 10. Preferred fermentation conditions for yeast and filamentous fungi are a temperature in the range of from about 25° C. to about 55° C. and at a pH of from about 3 to about 9. The appropriate conditions are usually selected based on the choice of host cell. Accordingly, in an embodiment the method of the invention further comprises one or more elements selected from:
a) culturing the cell culture in a nutrient medium;
b) culturing the cell culture under aerobic or anaerobic conditions
c) culturing the cell culture under agitation;
d) culturing the cell culture at a temperature of between 25 to 50° C.;
e) culturing the cell culture at a pH of between 3-9; and
f) culturing the cell culture for between 10 hours to 30 days.

The target compound(s) of the invention may be recovered and or isolated using methods known in the art. For example, the compound(s) may be recovered from the nutrient medium by conventional procedures including, but not limited to, collection, centrifugation, filtration, extraction, spray-drying, evaporation, or precipitation. The compound may be isolated by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989). In a particular embodiment the recovering and/or isolation step of the method of the invention comprises separating a liquid phase of the host cell or cell culture from a solid phase of the host cell or cell culture to obtain a supernatant comprising the at least one target compound and subjecting the supernatant to one or more steps selected from:

a) contacting the supernatant with one or more adsorbent resins in order to obtain at least a portion of the produced target compound;

b) contacting the supernatant with one or more ion exchange or reversed-phase chromatography columns in order to obtain at least a portion of the target compound; and

c) crystallizing or extracting the target compound from the supernatant; and

d) evaporating the solvent of the from the supernatant to concentrate or precipitate the target compound;
thereby recovering and/or isolating the target compound.

Not all conversion steps of pathway to produce the target compound of the invention need to occur in vivo in the host cell, so in a particular embodiment one or more of these steps are carried out in vitro. Accordingly, in an embodiment the method of the invention comprises at least one pathway step which is performed in vitro. Preferred target compounds to be produced using the method of the invention are listed supra.

### Fermentation Liquids

The invention also provides a fermentation liquid comprising the at least one target compound of the invention comprised in the cultivated cell culture of the invention. Preferably, at least 50%, such as at least 75%, such as at least 95%, such as at least 99% of the host cells of the culture are lysed and preferably at least 50%, such as at least 75%, such as at least 95%, such as at least 99% of solid cellular material has separated from the liquid. In an embodiment the fermentation liquid further comprises one or more compounds selected from:
a) Precursor or products of the operative biosynthetic metabolic pathway producing the at least one target compound of the invention;
b) supplemental nutrients comprising trace metals, vitamins, salts, yeast nitrogen base, YNB, and/or amino acids; and
wherein the concentration of the compound of the invention at least 1 mg/L fermentation liquid.

Preferably, the concentration of the at least target compound in the fermentation liquid is at least 5 mg/L, such as at least 10 mg/L, such as at least 20 mg/l, such as at least 50 mg/L, such as at least 100 mg/L, such as at least 500 mg/L, such as at least 1000 mg/L, such as at least 5000 mg/L, such as at least 10000 mg/L, such as at least 50000 mg/L.

### Compositions

In a further aspect the invention provides a composition comprising the fermentation liquid of the invention and one or more agents, additives and/or excipients. Agents, additives and/or excipients includes formulation additives, stabilising agent and fillers.

The composition of the invention may be formulated into a dry solid form by using methods known in the art. Further, the composition may be in dry form such as a spray dried, spray cooled, lyophilized, flash frozen, granular, microgranular, capsule or microcapsule form made using methods known in the art.

The composition of the invention may also be formulated into liquid stabilized form using methods known in the art. Further, the composition may be in liquid form such as a stabilized liquid comprising one or more stabilizers such as sugars and/or polyols (e.g. sugar alcohols) and/or organic acids (e.g. lactic acid).

### Pharmaceutical Preparations

The invention further provides a method for preparing a pharmaceutical preparation comprising subjecting a composition of the invention to one or more steps of converting the target compound of the invention in the composition to a pharmaceutically active derivative selected from the group consisting of Berberine, Papaverine, Morphine, Sanguinarine, Noscapine, Neomorphine, hydrocodone, Codeine, Oxycodone, Oxymorphone, Dihydromorphine and buprenorphine; and mixing the derivative with one or more pharmaceutical grade additives and/or adjuvants. The target compound of the invention may be converted by chemical conversion, by in vitro enzymatic conversion or by in vivo enzymatic conversion or any combination of the said conversion methodology. In one embodiment the compound of the invention is thebaine and the thebaine is converted to a pharmaceutically active thebaine derivative selected from the group consisting of Morphine, neomorphine, hydrocodone, Codeine, Oxycodone, Oxymorphone, Dihydromorphine, etorphine and buprenorphine. In another embodiment the compound of the invention is(S)-norcoclaurine and the(S)-norcoclaurine is converted to a pharmaceutically active derivative selected from the group consisting of Berberine, Papaverine, Sanguinarine, and Noscapine.

### Method of Use

The invention further provides a pharmaceutical preparation obtainable or obtained from the method of the invention converting the compound of the invention into a pharmaceutically active derivative. The pharmaceutical preparation may be used as a medicament to treat alleviate a disease or pathological conditions, particularly in a mammal. The pharmaceutical preparation may be used as an analgesic, an antimicrobial, an antitussive, an antiparasitic, an cytotoxic, an antipoisoning and/or an anticancer agent. In addition, the invention also provides a method for treating pain, infectious conditions, tussive conditions, parasitic conditions, cytotoxic conditions, opiate poisoning conditions and/or cancerous conditions in a mammal comprising administering a therapeutically effective amount of the pharmaceutical preparation of the invention to the mammal. The mammal is preferably a human, a livestock and/or pet animal.

### EXAMPLES

### Materials and Methods

### Materials

Chemicals used in the examples herein e.g. for buffers and substrates are commercial products of at least reagent grade.

### Strains

S288C is a common strain of S. cerevisiae available eg. from American Type Culture Collection (ATCC #204508™).

The S. cerevisiae strain (BY4741) used throughout these examples can be derived from S288C using the methodology of Brachmann C B, et al.; Yeast 14(2):115-32; 1998 and/or Winston F, et al.; Yeast 11(1):53-5; 1995. BY4741 strains can also be obtained commercially from ATTC or EUROSCARF.

### Example 1—Analytical Procedures

Metabolites were separated and identified by reversed-phase UPLC-MS using an Agilent 1290 UPLC coupled to an Ultivo Triple Quadrupole using the following settings:
Mobile Phase A. 0.1% aqueous solution of formic acid;
Mobile Phase B: 0.1% solution of formic acid in Acetonitrile;
Column: Kinetex™ 1.7 μm XB-C18 100 Å, 2.1×100 mm from Phenomenex™.

The elution gradient shown in Table 1 was used with the UPLC conditions shown in Table 2. Table 3 shows the mass spectrometer settings and parameters used and table 4 shows the target compound, retention time, parent ion, transition ions (MRM) as well as dwell time, fragmentor voltage, and collision energy used.

TABLE 1
Gradient for UPLC

| Time (min) | % B |
|---|---|
| 0 | 2 |
| 0.30 | 2 |
| 3.00 | 25 |
| 3.40 | 100 |
| 3.90 | 100 |
| 4 | 2 |
| 5 | 2 |

TABLE 2
UPLC conditions

| Parameter | Setting |
|---|---|
| Injection volume | 2 μl |
| Column Temperature | 30° C. ± 4° C. |
| Injection method | Flow through needle |
| Flow | 0.4 mL/min |
| Auto sampler temperature | 10° C. ± 2° C. |
| Reconditioning wash | 2% Acetonitrile (in H₂O), 5 sec |
| Weak wash | 20% Methanol (in H₂O), 5 sec |
| Strong wash | 30% Acetonitrile, 30% Methanol, |
| | 30% 2-propanol, 10% H₂O, 10 sec |
| Seal wash | 20% 2-Propanol (in H₂O) |

TABLE 3
Mass spectrometer source and detector settings
(Ultivo Triple Quadrupole)

| Source Parameter | Setting |
|---|---|
| Ion Source | Electrospray Positive Mode (ESI+) |
| Capillary Voltage | 3.5 kV |
| Nozzle Voltage | 500 V |
| Source Gas Temperature | 290° C. |
| Source Gas Flow | 12 L/min |
| Source Sheath Gas Temperature | 380° C. |
| Source Sheath Gas Flow | 12 L/min |
| Nebulizer | 30 psi |
| Mode | MS/MS |
| Collision | See Table 4 |

TABLE 4
Multiple reaction monitoring targets and conditions (ESI+)

| | Retention | Parent | Daughter | Dwell | Fragmentor | Collision |
|---|---|---|---|---|---|---|
| Target | time | ion | ion | time | voltage | energy |
| compound | (min) | (m/z) | (m/z) | (ms) | (V) | (V) |
| Dopamine | 0.7 | 154 | 137 | 200 | 110 | 5 |
| Norcoclaurine | 2.29 | 272 | 255 | 200 | 110 | 5 |

### Example 2—Construction of a Saccharomyces cerevisiae Strain for Production of Dopamine and Norcoclaurine

A BY4741 S. cerevisiae strain was modified to delete the native gene ARI1; YGL157W; SGD:S000003125 as disclosed in the saccharomyces genome database (SGD), by replacing the ORF encoding ARI1 with the KanMX dominant selection marker cassette (see Walker, M E et al.; FEMS Yeast Res. 2003 December; 4 (3): 339-47).

This strain was further modified to express an N-terminally truncated Coptis japonica Norcoclaurine Synthase (d19CjNCS—SEQ ID NO: 75). The truncation replaced the first 19 amino acids of SEQ ID NO: 75 with a methionine thereby removing a putative signal peptide. The gene was expressed using the well known S. cerevisiae PGK1 promoter, and the expression cassette was integrated in site XII-2 with the gene HIS3 as selection marker for growth on media lacking histidine (described by Mikkelsen, M D et al. (Metab. Eng. 14, Issue 2, 104-111 (2012)).

Using the S. cerevisiae gene integration and expression system developed by Mikkelsen, M D et al. (Metab. Eng. 14, Issue 2, 104-111 (2012)) (genes synthesized by Twist Bioscience, San Francisco, CA, USA, expression cassettes containing genes encoding the Pseudomonas putida DOPA decarboxylase (PpDODC-SEQ ID NO: 60), a Beta vulgaris CYP450 reductase (BvCPR1-SEQ ID NO: 76), a feed-back resistant S. cerevisiae ARO7 (ARO7fbr-SEQ ID NO: 77) and a gene encoding a CYP450 family 76 protein (SEQ ID NO: 2, 4, 6, 8, 10, 12, 14, 16, 18, 20, 22, 24, 26, 28. 30, 32, 34, 36, 38, 40, 42, 44, 46, 48, 50, 52, 54, 56 or 58) were integrated into the site XI-5 of the ARI1 deleted S. cerevisiae strain with Norcoclaurine Synthase expression described above. The twenty-nine CYP450s were tested separately for tyrosine hydroxylase activity in the strain background described. The genes were selected for testing based upon sequence homology to a double mutant to the CYP76AD1 from Beta vulgaris (CYP76AD1, W13L, F309L—SEQ ID NO: 58—also published in WO16049364) and the activities of the strains were compared to a strain containing the double mutant protein. Selection for transformants was done using the well known Kluyveromyces lactis LEU2 marker available e.g. from EUROSCARF and growth on media lacking leucine. Where all enzymes where expressed and active the recombinant S. cerevisiae strain produced norcoclaurine as the end product.

### Example 3 Production of Norcoclaurine and Dopamine Using Different CYP76 Tyrosine Hydroxylases

The recombinant S. cerevisiae transformants of example 2 were grown in triplicate in 96 deep-well plates in 500 μL liquid of the well known synthetic complete (SC) media available e.g. from Sigma Aldrich lacking histidine and leucine, for 3 days at 30° C. with shaking at 230 rpm in a Kuhner Climo-Shaker ISF1-X. Culture samples for LC-MS were prepared by extraction as follows: 96% ethanol and culture sample were mixed 1:1 and incubated on a heating block at 80° C. for 10 min. After heating, cells were pelleted in an Eppendorff tabletop centrifuge by centrifugation and the supernatant was then transferred to a new tube and diluted 1:5 in water.

As can be seen in table 5 (average of triplicate measurements), a number of tyrosine hydroxylases (CYP450 of family 76 (CYP76)) showed surprisingly good capabilities of producing L-dopa, dopamine and(S)-norcoclaurine when co-expressed with the DOPA decarboxylase (PpDODC-SEQ ID NO: 60) and Norcoclaurine Synthase (d19CjNCS-SEQ ID NO: 75) as described above. For several of the tested CYP76's production of L-dopa, dopamine and(S)-norcoclaurine was unexpectedly high compared to the modified CYP76AD1 disclosed by DeLoache, W. C. et al Nat. Chem. Biol., 11, 465-471 (2015), most notably by the spinach CYP76 SoCYP76ADr9. Other tested CYP450s are also more active than the modified CYP76AD1 as shown by the larger production of dopamine and/or norcoclaurine. Accordingly, it is contemplated that these CYP450s are tyrosine hydroxylases and not also cyclo-dopa synthases like the native BvCYP76AD1 from B. vulgaris described in WO16049364 A3.

TABLE 5

| Tyrosine Hydroxylase | | | Norcoclaurine | Dopamine |
|---|---|---|---|---|
| CYP76 | Sequence | Species | mg/l | AUC |
| AnCYP76ADr17 | SEQ ID NO: 10 | Abronia nealleyi | 0.729 | 3991 |
| SoCYP76ADr9 | SEQ ID NO: 2 | Spinacia oleracea | 0.650 | 15010 |
| BvCYP76ADr10 | SEQ ID NO: 8 | Beta vulgaris | 0.647 | 5009 |
| BvCYP76ADr8 | SEQ ID NO: 12 | Beta vulgaris | 0.633 | 2897 |
| BvCYP76ADr7 | SEQ ID NO: 14 | Beta vulgaris | 0.517 | 2745 |
| BvCYP76ADr6 | SEQ ID NO: 16 | Beta vulgaris | 0.496 | 2535 |
| OfCYP76ADr12 | SEQ ID NO: 4 | Opuntia ficus-indica | 0.477 | 9549 |
| FICYP76ADr11 | SEQ ID NO: 6 | Froelichia latifolia | 0.388 | 9301 |
| AoCYP76ADr16 | SEQ ID NO: 24 | Acleisanthes obtuse | 0.250 | 1080 |
| BvCYP76AD1 | SEQ ID NO: 58 | Beta vulgaris | 0.242 | 1109 |
| PdCYP76ADr21 | SEQ ID NO: 22 | Phytolacca dioica | 0.145 | 1113 |
| MmCYP76ADr18 | SEQ ID NO: 26 | Mirabilis multiflora | 0.142 | 816 |
| EvCYP76ADr20 | SEQ ID NO: 20 | Ercilla volubilis | 0.125 | 1236 |
| PaCYP76ADr19 | SEQ ID NO: 32 | Phytolacca americana | 0.076 | 405 |
| CbCYP76ADr28 | SEQ ID NO: 18 | Cleretum bellidiforme | 0.075 | 2032 |
| AoCYP76ADr24 | SEQ ID NO: 28 | Acleisanthes obtuse | 0.075 | 739 |
| AnCYP76ADr27 | SEQ ID NO: 30 | Abronia nealleyi | 0.062 | 530 |
| CqCYP76ADr5 | SEQ ID NO: 34 | Chenopodium quinoa | 0.053 | 260 |
| CqCYP76ADr4 | SEQ ID NO: 38 | Chenopodium quinoa | 0.034 | 209 |
| MmCYP76ADr22 | SEQ ID NO: 36 | Mirabilis multiflora | 0.032 | 238 |
| AnCYP76ADr23 | SEQ ID NO: 42 | Abronia nealleyi | 0.008 | 139 |
| PaCYP76ADr14 | SEQ ID NO: 40 | Phytolacca americana | 0.008 | 157 |
| CqCYP76ADr13 | SEQ ID NO: 50 | Chenopodium quinoa | 0.000 | 54 |
| MjCYP76ADr26 | SEQ ID NO: 54 | Mirabilis jalapa | 0.000 | 12 |
| MmCYP76ADr25 | SEQ ID NO: 56 | Mirabilis multiflora | 0.000 | 10 |
| Neg K | — | — | 0.000 | 8 |
| SoCYP76ADr1 | SEQ ID NO: 48 | Spinacia oleracea | 0.000 | 71 |
| SoCYP76ADr15 | SEQ ID NO: 52 | Spinacia oleracea | 0.000 | 30 |
| SoCYP76ADr2 | SEQ ID NO: 44 | Spinacia oleracea | 0.000 | 111 |
| SoCYP76ADr3 | SEQ ID NO: 46 | Spinacia oleracea | 0.000 | 98 |

1. A recombinant microbial host cell comprising an operative biosynthetic metabolic pathway capable of producing one or more target compounds selected from the group consisting of L-dopa, dopamine, (S)-Norcoclaurine, and derivatives thereof;
wherein said pathway comprises one or more heterologous L-tyrosine hydroxylases (TyrH) capable of converting L-Tyrosine into L-dopa and capable of increasing the cell production of the one or more target compounds compared to a reference L-tyrosine hydroxylase having the sequence set forth in SEQ ID NO: 58, wherein the one or more heterologous TyrH is a polypeptide having at least 70%, such as at least 75%, such as at least 80%, such as at least 90%, such as at least 95%, such as at least 99%, such as at least 100% sequence identity to SEQ ID NO: 4.

2. The host cell of claim 1, wherein the operative biosynthetic metabolic pathway further comprises one or more enzymes of a benzylisoquinoline alkaloid (BIA) pathway.

3. The host cell of claim 2, wherein the one or more enzymes of the BIA pathway is selected from the group consisting of:
a) 3-deoxy-D-arabino-2-heptulosonic acid 7-phosphate (DAHP) synthase;
b) 5-enolpyruvylshikimate-3-phosphate (EPSP) synthase
c) chorismate synthase;
d) chorismate mutase, wherein the chorismate mutase has at least 70% sequence identity to SEQ ID NO: 77;
e) prephenate dehydrogenase;
f) aromatic aminotransferase;
g) P450 reductase (CPR), wherein the CPR has at least 70% sequence identity to SEQ ID NO: 76;
h) L-dopa decarboxylase (DODC), wherein the DODC has at least 70% sequence identity to SEQ ID NO: 60;
i) Tyrosine decarboxylase (TYDC);
j) hydroxyphenylpyruvate decarboxylase (HPPDC);
k) Norcoclaurin synthase (NCS), wherein the NCS has at least 70% sequence identity to SEQ ID NO: 61 or SEQ ID NO: 75;
l) 6-O-methyltransferase (6-OMT), wherein the 6-OMT has at least 70% sequence identity to SEQ ID NO: 62;
m) Coclaurine-N-methyltransferase (CNMT), wherein the CNMT has at least 70% sequence identity to SEQ ID NO: 63;
n) N-methylcoclaurine 3′-monooxygenase (NMCH), wherein the NMCH has at least 70% sequence identity to SEQ ID NO: 65;
o) 3′-hydroxy-N-methyl-(S)-coclaurine 4′-O-methyltransferase (4′-OMT), wherein the 4′-OMT has at least 70% sequence identity to SEQ ID NO: 66;
p) 1,2-dehydroreticuline synthase-1,2-dehydroreticuline reductase (DRS-DRR), wherein the DRS-DRR has at least 70% sequence identity to SEQ ID NO: 68;
q) salutaridine synthase (SAS), wherein the SAS has at least 70% sequence identity to SEQ ID NO: 70;
r) salutaridine reductase (SAR), wherein the SAR has at least 70% such as at least 99%, such as at least 100% sequence identity to SEQ ID NO: 71;
s) salutaridinol 7-O-acetyltransferase (SAT), wherein the SAT has at least 70%, sequence identity to SEQ ID NO: 73; and
t) Thebaine synthase (THS), wherein the THS has at least 80% sequence identity to SEQ ID NO: 79 or SEQ ID NO: 80.

4. The host cell of claim 1, wherein the one or more target compounds is a benzylisoquinoline alkaloid.

5. The host cell of claim 4, wherein the benzylisoquinoline alkaloid is selected from the group consisting of:
a) (S)-Norcoclaurine;
b) (S)-Norlaudanosoline;
c) (S)-Coclaurine;
d) (S)-3′-Hydroxy-coclaurine;
e) (S)-N-Methylcoclaurine;
f) (S)-3′-Hydroxy-N-Methylcoclaurine;
g) (S)-Reticuline;
h) (R)-Reticuline;
i) Salutaridine;
j) Salutaridinol; and
k) Thebaine.

6. The host cell of claim 5, wherein the benzylisoquinoline alkaloid is Thebaine.

7. The host cell of claim 1, wherein the host cell is a yeast cell.

8. The host cell of claim 7, wherein the yeast cell is a S. cerevisiae.

9. The host cell of claim 7, wherein the yeast cell is a S. cerevisiae strain modified by deletion, disruption or downregulation of the native gene ARI1.

10. A cell culture, comprising the host cell of claim 1 and a fermentation liquid.

11. A method for producing at least one target compound selected from the group consisting of one or more of L-dopa, dopamine, (S)-Norcoclaurine, and derivatives thereof comprising:
a) culturing the cell culture of claim 10 at conditions allowing the host cell to produce the at least one target compound; and
b) optionally recovering and/or isolating the at least one target compound.

12. The method of claim 11, wherein at least one step of producing the at least one target compound is performed in vitro.

13. The method of claim 11, wherein the at least one target compound is a benzylisoquinoline alkaloid selected from the group consisting of:
a) (S)-Norcoclaurine;
b) (S)-Norlaudanosoline;
c) (S)-Coclaurine;
d) (S)-3′-Hydroxy-coclaurine;
e) (S)-N-Methylcoclaurine;
f) (S)-3′-Hydroxy-N-Methylcoclaurine;
g) (S)-Reticuline;
h) (R)-Reticuline;
i) Salutaridine;
j) Salutaridinol; and
k) Thebaine.

14. A fermentation liquid comprising the cell culture of claim 10 and at least one target compound selected from L-dopa, dopamine, (S)-Norcoclaurine, and derivatives thereof comprised in the cell culture of claim 10.

15. The fermentation liquid of claim 14, further comprising one or more compounds selected from:
a) precursor or products of the operative biosynthetic metabolic pathway producing the at least one target compound;
b) supplemental nutrients comprising; and
wherein the concentration of the at least one target compound is at least 1 mg/l liquid.

16. A composition comprising the fermentation liquid of claim 14 and one or more agents, additives and/or excipients.

17. A method for preparing a pharmaceutical preparation comprising subjecting the composition of claim 16 to one or more steps of converting the target compound in the composition to a pharmaceutically active derivative selected from the group consisting of Berberine, Papaverine, Morphine, Sanguinarine, Noscapine, Neomorphine, hydrocodone, Codeine, Oxycodone, Oxymorphone, Dihydromorphine, and buprenorphine; and mixing the pharmaceutically active derivative with one or more pharmaceutical grade additives and/or adjuvants.

18. The method of claim 17, wherein the target compound is converted by chemical conversion, by in vitro enzymatic conversion or by in vivo enzymatic conversion or any combination of the said conversions.


SEQUENCE LISTING (80 sequences)

SEQ ID NO: 1 - DNA, 1500 bp, Spinacia oleracea
  source 1..1500: mol_type = other DNA
atggaaaacaccaccttggccttgattttgccaattttgttcatctgcttccacctgatccattccttcatttgccaatccagaaagtcctctaaattgccaccaggtccaaaaagaatgccaatctttggtaacatcttcgacttgggtgaaaagccacatagatcttttgctaacttggctaagattcacggtccattggtttctttgaagttgggttctatcactaccatcgttgtttcatcagctgaagttgccaaagaaatgttcttgaagaacgaccagttgttgtccaacagaactattccagattctgttagagctggtgatcacgataagttgtctatgtcttggttgccagtttctgctaaatggcgtaacttgagaaagatttctgctgtgcagctgttgtctaatcaaagattagatgcttcccaagctcaaagacaagctaaagtcaaacagttgttggcttacgttcaagactgttctaaaaagggtcaaccagttgatattggtagagctgcttttactacctccttgaacttgttgtcaaacaccttcttctctgttgaattggcctctcatgaatcctctgtttctcaagaattcaagcagttgatgtggaacatgatggaagaaattggtagaccaaactacgctgattacttcccaattttgggttacgttgatcctttcggtatcagaagaagattggctgcttactttgaccaattgatcgttgtgttccaagacatcatcagagaaagacaaaaagtcaggtctacgaatggttctaacgctaagcaaactaacgatatcttggacaccttgttgaacttgcatggtgaaaacgaattgtccatgggtgaaatcaatcatttgttggccgatattttcaacgctggtactgatacaactgcttctactttggaatgggctatgactgaattggttaagaacccaaatatgatgggtagagtccagaacgaaattgaacaagctttgggtagagattgctcctccattcaagaatctgacatcttgaaattgccatacttgcaggccattatcaaagagactttgagattgcatccaccaaccgtttttttgttgccaagaaaagctgatactgacgtcgaattgaacggttatttggttccaaagaacgcccaagttttggttaatttgtgggctataggtagagatccaaaggtttgggaaaatccagaagttttcttgccagaacgtttcttgaactccgaaattgatgttaagggcagagattttgagttgttgccatttggtgctggtagaagaatttgtccaggtttgacattggcttacaggatgttgaatttgatgttggctaccttggttaacaactacgattggaaattggaggatggtatgaacccagaaaatttggatatggacgaaaagttcggtattaccttgcaaaaggttaacccattgagagccattccaattccaagatga

SEQ ID NO: 2 - protein, 499 aa, Spinacia oleracea
MENTTLALILPILFICFHLIHSFICQSRKSSKLPPGPKRMPIFGNIFDLGEKPHRSFANLAKIHGPLVSLKLGSITTIVVSSAEVAKEMFLKNDQLLSNRTIPDSVRAGDHDKLSMSWLPVSAKWRNLRKISAVQLLSNQRLDASQAQRQAKVKQLLAYVQDCSKKGQPVDIGRAAFTTSLNLLSNTFFSVELASHESSVSQEFKQLMWNMMEEIGRPNYADYFPILGYVDPFGIRRRLAAYFDQLIVVFQDIIRERQKVRSTNGSNAKQTNDILDTLLNLHGENELSMGEINHLLADIFNAGTDTTASTLEWAMTELVKNPNMMGRVQNEIEQALGRDCSSIQESDILKLPYLQAIIKETLRLHPPTVFLLPRKADTDVELNGYLVPKNAQVLVNLWAIGRDPKVWENPEVFLPERFLNSEIDVKGRDFELLPFGAGRRICPGLTLAYRMLNLMLATLVNNYDWKLEDGMNPENLDMDEKFGITLQKVNPLRAIPIPR

SEQ ID NO: 3 - DNA, 1485 bp, Opuntia ficus-indica
  source 1..1485: mol_type = other DNA
atggatcatgctattttggctaccttggtctgcattttgttcgtttgttttcatttgttgtggcgtaggtcctacaagaactcttctaaattgccaccaggtccaaaaccagttccaatttttggtaacatcttcgagttgggtgaaaagccacatagatcttttgctgatttggccaagattcacggtccattgatttctttgaaattgggttctgtcacgacgatcgttgtttcttcttctgatgttgccaaagaaatgttcttgaagcacgatcaagttttcgccaacagaactattccagattctgttagagctggtaaccacgataagttgtctatgtcttggttgccagtttctgctaaatggcgtaacatgagaaagatttctgctgtccaactgttgtctacccaaagattggattctaatcaaggtttgagacaagccaaggttcaacagttgttggaatacgttcaagaatgttgcaaaaagggtcaaccagttgatattggtagagctgcttttactacctccttgaacttgttgtctaacaccttcttctctatggaattggctcaacattcatcctctgcttctcaagaattcaagcaattgatgtggtgcatcatggaagaaattggtagaccaaattacgccgattacttcccaattttgggttactttgatccattcggtatcagacgtagattgactgcttacttcgacaagttgattgccatcttccaagatatcatccacgaaagattgaaggctagatctactggttcctcttctaccaacgatattttggacactctgctgaacttgtaccaagaaaacgaattgtctatggacgagatcaaccacttgttggttgatatttttgatgccggtactgataccactgcttctactttggaatgggctatggctgaattggttaagaacccagaaattatgattaaggcccaggatgagattaacgctgctttgggtaaagattgctccgttatccatgaatccgatatcgttaagttgccatacttgcaagccattgtcaaagaaaccttgagattgcatccaccaactgtttttttgttgcctagaaaagccgatttggatgttgagttgtatggttacgttgttccaaagaacgcccaaatcttggttaatttgtgggctataggtagagatccaaaggtttggtctaatccagaagttttctcaccagaaagattcttggactctaccattgatgttaagggtagagattttgagttgttgccatttggtgcaggtagaagaatttgtccaggtttgactttggcttccagaatgttgaatttgatgttggccactttggtccataacttcaactggaaattagaggatggtatgatcccaaaggatttggatatgactgaaaagttcggtattaccttgcagaaggttaacccattgcaagttattcccatccaaaagtaa

SEQ ID NO: 4 - protein, 494 aa, Opuntia ficus-indica
MDHAILATLVCILFVCFHLLWRRSYKNSSKLPPGPKPVPIFGNIFELGEKPHRSFADLAKIHGPLISLKLGSVTTIVVSSSDVAKEMFLKHDQVFANRTIPDSVRAGNHDKLSMSWLPVSAKWRNMRKISAVQLLSTQRLDSNQGLRQAKVQQLLEYVQECCKKGQPVDIGRAAFTTSLNLLSNTFFSMELAQHSSSASQEFKQLMWCIMEEIGRPNYADYFPILGYFDPFGIRRRLTAYFDKLIAIFQDIIHERLKARSTGSSSTNDILDTLLNLYQENELSMDEINHLLVDIFDAGTDTTASTLEWAMAELVKNPEIMIKAQDEINAALGKDCSVIHESDIVKLPYLQAIVKETLRLHPPTVFLLPRKADLDVELYGYVVPKNAQILVNLWAIGRDPKVWSNPEVFSPERFLDSTIDVKGRDFELLPFGAGRRICPGLTLASRMLNLMLATLVHNFNWKLEDGMIPKDLDMTEKFGITLQKVNPLQVIPIQK

SEQ ID NO: 5 - DNA, 1496 bp, Froelichia latifolia
  source 1..1496: mol_type = other DNA
tggacaacactaccttggccgttgttttgtctgttatgttcgttttgttccacctgttgaaaaccatcttcaccaactcttctaacaacaaattgccaccaggtccaaagagaatgccaatttttggtaacatcttcgacttgggtgaaaagccacatagatcttttgctaacttcgccaaaattcacggtccattgatttctttgaagttgggttctgttacgacgatcgttgtttcttctgcttctgttgctgaagagatgttcttgaagaatgatcaagctttggccaacagaaccattccagattctgttagagctggtgatcatgacaaattgtctatgtcttggttgccagtttctgctaaatggcgtaacttgagaaagatttctgctgtccagttgttgtctaaccagagattggatgcttctcaaccattgagacaagctaaagttaagcagttgttggcctacgttcaaaactgctctgaaaagaatcaagccgttgatattggtagagctgctttcactacttccttgaacttgttgtccaacaccttcttctctatggaattggcttctcatgaatcctctgcttcccaagaattcaaacaattgatgtggaacatcatggaagaaatcggtagaccaaattacgccgattttttcccaatcttgggttacattgatccattcggtatcagaagaagattggctggttacttcgataagttgatcgatgttttccaggacatcatcagagaaagacaaaagattagagcctctaacgctaacggtactaagcaaacttctgatatcttggacaccctgttgaagttgtacgaagataacgaattatccatgggtgagatcaaccacttgttggttgatatttttgatgccggtactgataccactgcttctactttggaatgggctatggctgaattggttaagaatccagaaatgatggttagagcccagaacgaaattgaagaagttttgggtaaagactgctccaacatccaagaatccgatatttctaagttgccatacttgcaggccatcatcaaagagtctttaagattgcatccaccaaccgtttttctgttgccaagaaaagctgatgttgatgtcgaattatacggttacgttgttccaaagaacgcccaagttttggttaatttgtgggctataggtagagatccaaaggtttggaaaaacccagaagttttctcaccagaaaggttcttggaatgcgatattgattacaagggtagagactttgagttgttgccatttggtgctggtagaagaatttgtccaggtttgactttggcttacaggatgttgaatttgatgttggctaccttgttgcacaactacaattggaaattgggtgatgatatggacccaaaggacttggatatggaagagaaatttggtatcaccctgcagaagattaagccattgcaagttattccagttgccaggtaa

SEQ ID NO: 6 - protein, 498 aa, Froelichia latifolia
MDNTTLAVVLSVMFVLFHLLKTIFTNSSNNKLPPGPKRMPIFGNIFDLGEKPHRSFANFAKIHGPLISLKLGSVTTIVVSSASVAEEMFLKNDQALANRTIPDSVRAGDHDKLSMSWLPVSAKWRNLRKISAVQLLSNQRLDASQPLRQAKVKQLLAYVQNCSEKNQAVDIGRAAFTTSLNLLSNTFFSMELASHESSASQEFKQLMWNIMEEIGRPNYADFFPILGYIDPFGIRRRLAGYFDKLIDVFQDIIRERQKIRASNANGTKQTSDILDTLLKLYEDNELSMGEINHLLVDIFDAGTDTTASTLEWAMAELVKNPEMMVRAQNEIEEVLGKDCSNIQESDISKLPYLQAIIKESLRLHPPTVFLLPRKADVDVELYGYVVPKNAQVLVNLWAIGRDPKVWKNPEVFSPERFLECDIDYKGRDFELLPFGAGRRICPGLTLAYRMLNLMLATLLHNYNWKLGDDMDPKDLDMEEKFGITLQKIKPLQVIPVAR

SEQ ID NO: 7 - DNA, 1497 bp, Beta vulgaris
  source 1..1497: mol_type = other DNA
atggataacgctaccttggccgttatcttgtctattttgttcgttttctaccacatcttcaagtcgttcttcaccaactcttcatctagaagattgccaccaggtccaaaaccagttccaatttttggtaacatcttcgacttgggtgaaaagccacatagatcttttgctaacttgtccaagattcacggtccattgatttctttgaagttgggttctgttacgacgatcgttgtttcttctgcttctgttgctgaagagatgttcttgaagaatgatcaagctttggccaacagaaccattccagattctgttagagctggtgatcatgacaaattgtctatgtcttggttgccagtttctcaaaagtggcgtaacatgagaaagatttctgctgttcagttgctgtccaatcaaaagttggatgcttctcaaccattgagacaagctaaagttaagcagttgttgtcctacgttcaagtctgctctgaaaaaatgcaaccagttgatattggtagagctgctttcactacttccttgaacttgttgtctaacaccttcttctccattgaattggcctctcatgaatcttcagcttcccaagaattcaagcaattgatgtggaacatcatggaagaaatcggtagaccaaattacgccgattttttcccaatcttgggttacattgatccattcggtatcagaagaagattggctggttacttcgataagttgatcgatgttttccaggacatcatcagagaaagacagaagttgagatcctctaattcttctggtgctaagcaaactaacgacatcttggatactttgttgaagttgcacgaagataacgagttgtctatgccagaaatcaaccacttgttggttgatattttcgatgctggtactgataccactgcttctactttggaatgggctatggctgaattggttaagaatccagaaatgatgaccaaggtccagatcgaaattgaacaagctttaggtaaggattgcttggacatccaagaatccgatatttctaagttgccatacttgcaggccatcatcaaagagactttgagattgcatccaccaaccgtttttttgttgccaagaaaggctgataacgatgttgagttgtatggttacgttgttccaaagaacgctcaagttttggttaacttgtgggctataggtagagatccaaaggtttggaaaaacccagaagttttctcaccagaaaggttcttggattgcaacattgattacaagggtagagacttcgagttgttgccatttggtgctggtagaagaatttgtccaggtttgactttggcttacaggatgttgaatttgatgttggctaccttgttgcagaactacaattggaaattggaggatggtatcaacccaaaggacttggatatggacgaaaagtttggtatcaccttgcaaaaggttaagccattgcaagttattcccgttccaagatga

SEQ ID NO: 8 - protein, 498 aa, Beta vulgaris
MDNATLAVILSILFVFYHIFKSFFTNSSSRRLPPGPKPVPIFGNIFDLGEKPHRSFANLSKIHGPLISLKLGSVTTIVVSSASVAEEMFLKNDQALANRTIPDSVRAGDHDKLSMSWLPVSQKWRNMRKISAVQLLSNQKLDASQPLRQAKVKQLLSYVQVCSEKMQPVDIGRAAFTTSLNLLSNTFFSIELASHESSASQEFKQLMWNIMEEIGRPNYADFFPILGYIDPFGIRRRLAGYFDKLIDVFQDIIRERQKLRSSNSSGAKQTNDILDTLLKLHEDNELSMPEINHLLVDIFDAGTDTTASTLEWAMAELVKNPEMMTKVQIEIEQALGKDCLDIQESDISKLPYLQAIIKETLRLHPPTVFLLPRKADNDVELYGYVVPKNAQVLVNLWAIGRDPKVWKNPEVFSPERFLDCNIDYKGRDFELLPFGAGRRICPGLTLAYRMLNLMLATLLQNYNWKLEDGINPKDLDMDEKFGITLQKVKPLQVIPVPR

SEQ ID NO: 9 - DNA, 1512 bp, Abronia nealleyi
  source 1..1512: mol_type = other DNA
atggaaaacaccaccttgggtgttattttggctactattttcttgaccttccacatcatgaagatgctgttgtctccaaacaaagctaaattgccaccaggtccaagaccattgccaattattggtaacattttggagttgggtgataagccacatagatcttttgctaacttggctaaaatctacggtccattgattaccttgagattgggttctgttactaccgttgttgtctcttcatctcaagttgccaaagaaatgttcttgaagaacgatcaatccttggccaacagaactattccagattctgttagagctggtaaccatcacaaattgtctatgtcttggttgccagtttctgctaagtggcgtaatttcagaaagatttctgccgttgagttgttgtcctctcaaagattggatgcttctcaagctcatagacaagctaaggttgaacagttgatcgaatacgtcaaagagtgctctaagattggtcaatgcgttgatattggtagattggctttcaccacttccttgaacttgttgtctaacaccttcttcagcaaagaattggcctctttggattctaacaacgcccaagaattcaagcaattgatgtggtgcattatggaagaaatcggtagaccaaattacgctgattacttcccaattttgggttacgttgatccattcggtgttagaagaagattgtccagatacttcgaccagttgattgaagtgttccaagaaatcatcagagagaggttgtctaagaacaacgaaaaggttgataacaagaacgacattttggccactttgctgcacttgtacaaacagaatgaattgtccatggatgagatcaaccacttgttggttgatattttcgatgctggtactgataccactgcttctactttggaatgggctatgtctgaattgatcaagaacccacatattatgtccaaggctcaagctgaagttagaagggctactgtttctcatggtggtgctactgttgctgctgttcaagaatctgatatttccaacttgccttacatccagtccatcatcaaagaaactttgagattgcatccaccaaccgtttttttgttgccaagaaaagctgatgttgacgttcagttgtacggttatgttgttccaaagaacgctcaagttttggttaacttgtgggcaattggtagagatccaaatgtttggccagatccagaagtttttagaccagaaaggtttatggattgcggtgttgatgttaagggtagagactttgaattattgccatttggtgccggtagaagaatttgtcctggtttgtctttggcttacaggatgttgaatctgatgttggctaatctggttcactctttcgattggaaattgccagattccggtaatggttttggttctgatcctaatggtttggacatggacgaaaagtttggtatcaccttgcaaaaagtcgaaccattgaaggttattccaacgccaagatga

SEQ ID NO: 10 - protein, 503 aa, Abronia nealleyi
MENTTLGVILATIFLTFHIMKMLLSPNKAKLPPGPRPLPIIGNILELGDKPHRSFANLAKIYGPLITLRLGSVTTVVVSSSQVAKEMFLKNDQSLANRTIPDSVRAGNHHKLSMSWLPVSAKWRNFRKISAVELLSSQRLDASQAHRQAKVEQLIEYVKECSKIGQCVDIGRLAFTTSLNLLSNTFFSKELASLDSNNAQEFKQLMWCIMEEIGRPNYADYFPILGYVDPFGVRRRLSRYFDQLIEVFQEIIRERLSKNNEKVDNKNDILATLLHLYKQNELSMDEINHLLVDIFDAGTDTTASTLEWAMSELIKNPHIMSKAQAEVRRATVSHGGATVAAVQESDISNLPYIQSIIKETLRLHPPTVFLLPRKADVDVQLYGYVVPKNAQVLVNLWAIGRDPNVWPDPEVFRPERFMDCGVDVKGRDFELLPFGAGRRICPGLSLAYRMLNLMLANLVHSFDWKLPDSGNGFGSDPNGLDMDEKFGITLQKVEPLKVIPTPR

SEQ ID NO: 11 - DNA, 1500 bp, Beta vulgaris
  source 1..1500: mol_type = other DNA
atggacaacactaccttggccttgatcttgtcatctttgttcgtttgcttccagttgatcaggtctttcattaaccataccaagaagtccaacaaattgccaccaggtccaaaaagaatgccaattttcggtaacatcttcgacttgggtgaaaagccacatagatcttttgctaacttggctaagattcacggtccattggtttctttacaattgggttctgttactaccgtcgttgtttcttctgctgatgttgctaaagaaatgttcttgaagaacgatcaagctttggccaacagaactattccagattctgttagagctggtgatcacgataagttgtctatgtcttggttgccagtttctgctaaatggcgtaacttgagaaagatttctgctgtccaactgttgtctacccaaagattggatgcttctcaagctcatagacaatctaaggttcaacagttgttggaatacgttcacgattgctctaaaaagggtcaaccagttgatattggtagagctgcttttactacctccttgaacttgttgtctaacaccttcttctctgttgaattggcctctcatgaatcttcagcttcccaagaattcaagcaattgatgtggaacatcatggaagaaatcggtagaccaaattacgccgattttttcccaattttgggttacttggatccattcggtatcagaagaagattggctggttacttcgaccaattgattgctgttttccaagacatcattggtgagagacaaaaaatcagatccgctaatttgtctggtggtaagcaaactaacgatatcttggataccctgctgaacttgtacgacgaaaaagaattatccatgggtgagatcaaccacttgttggttgatatttttgatgccggtactgataccactgcttctactttggaatgggctatggctgaattggttaagaacccatatatgatggttaaggtccaggacgaaattgaaaaggctattggtaaaggttgctccatggttcaagaatccgatatttctaagttgccatacttgcaggccatcatcaaagaaactttgagattgcatccaccaaccgtttttttgttgccaagaaaagctgatgcagatgttgagttgtatggttacatcgttccaaagaatgctcaagtcttggttaacttgtgggctataggtagagatccaaaggtttggaaaaacccagaagttttctcaccagaaaggttcttggaatccaacattgattacaagggtagagactttgagttgttgccatttggtgctggtagaagaatttgtccaggtttgactttggcttacaggatgttgaacttaatgatggccaacttcttgcactcttacgattggaaattggaagatggtatgcaccctaaggatttggatatggacgaaaagtttggtatcaccttgcaaaaggttaagccattgcaagttattccagtgccaagatga

SEQ ID NO: 12 - protein, 499 aa, Beta vulgaris
MDNTTLALILSSLFVCFQLIRSFINHTKKSNKLPPGPKRMPIFGNIFDLGEKPHRSFANLAKIHGPLVSLQLGSVTTVVVSSADVAKEMFLKNDQALANRTIPDSVRAGDHDKLSMSWLPVSAKWRNLRKISAVQLLSTQRLDASQAHRQSKVQQLLEYVHDCSKKGQPVDIGRAAFTTSLNLLSNTFFSVELASHESSASQEFKQLMWNIMEEIGRPNYADFFPILGYLDPFGIRRRLAGYFDQLIAVFQDIIGERQKIRSANLSGGKQTNDILDTLLNLYDEKELSMGEINHLLVDIFDAGTDTTASTLEWAMAELVKNPYMMVKVQDEIEKAIGKGCSMVQESDISKLPYLQAIIKETLRLHPPTVFLLPRKADADVELYGYIVPKNAQVLVNLWAIGRDPKVWKNPEVFSPERFLESNIDYKGRDFELLPFGAGRRICPGLTLAYRMLNLMMANFLHSYDWKLEDGMHPKDLDMDEKFGITLQKVKPLQVIPVPR

SEQ ID NO: 13 - DNA, 1503 bp, Beta vulgaris
  source 1..1503: mol_type = other DNA
atggacaacactaccttggccttgatcttgtcatctttgttcgtttgcttccagttgatcaggtcctttattaaccatgccaagaagtctaacaaattgccaccaggtccaaagagaatgccaatttttggtaacatcttcgacttgggtgaaaagccacatagatcttttgctaacttggctaagattcacggtccattggtttctttacaattgggttctgttactaccgtcgttgtttcttctgctgatgttgctaaagaaatgttcttgaagaacgatcaagctttggccaacagaactattccagattctgttagagctggtgatcacgataagttgtctatgtcttggttgccagtttctgctaaatggcgtaacttgagaaagatttctgctgtccaactgttgtctacccaaagattggatgcttctcaagctcatagacaatctaaggttcaacagttgttggaatacgttcacgattgctctaaaaagggtcaaccagttgatattggtagagctgcttttactacctccttgaacttgttgtctaacaccttcttctctgttgaattggcctctcatgaatcttcagcttcccaagaattcaagcaattgatgtggaacatcatggaagaaatcggtagaccaaattacgccgattttttcccaattttgggttacttggatccattcggtatcagaagaagattggctggttacttcgaccaattgattgctgttttccaagacatcattggtgagagacaaaaaatcagatccgctaatttgtctggtggtaagcaaactaccaacgatattttggataccctgctgaacttgtacgacgagaaagaattatctatgggtgagatcaaccacttgttggttgatatttttgatgccggtactgataccactgcttctactttggaatgggctatggctgaattggttaagaatccagatatgatggttaaggtccaggacgaaattgaacaagctattggtaaaggttgctccatggttcaagaatctgatatctctaagttgccatacttgcaggccattatcaaagaaaccttgagattgcatccaccaaccgtttttttgttgccaagaaaagctgatgcagatgttgagttgtatggttacgttgttccaaagaatgctcaagtcttggttaacttgtgggctataggtagagatccaaaggtttggaaaaacccagaagttttctcaccagaaaggttcttggaatccaacattgattacaagggtagagactttgagttgttgccatttggtgctggtagaagaatttgtccaggtttgactttggcttacaggatgttgaatttgatgatggccaacttcttgcactcttacgattggaaattggaagatggtatgcaccctaaggatttggatatggacgaaaagtttggtatcaccttgcaaaaggttaagccattgcaagttattccagtgccaagatga

SEQ ID NO: 14 - protein, 500 aa, Beta vulgaris
MDNTTLALILSSLFVCFQLIRSFINHAKKSNKLPPGPKRMPIFGNIFDLGEKPHRSFANLAKIHGPLVSLQLGSVTTVVVSSADVAKEMFLKNDQALANRTIPDSVRAGDHDKLSMSWLPVSAKWRNLRKISAVQLLSTQRLDASQAHRQSKVQQLLEYVHDCSKKGQPVDIGRAAFTTSLNLLSNTFFSVELASHESSASQEFKQLMWNIMEEIGRPNYADFFPILGYLDPFGIRRRLAGYFDQLIAVFQDIIGERQKIRSANLSGGKQTTNDILDTLLNLYDEKELSMGEINHLLVDIFDAGTDTTASTLEWAMAELVKNPDMMVKVQDEIEQAIGKGCSMVQESDISKLPYLQAIIKETLRLHPPTVFLLPRKADADVELYGYVVPKNAQVLVNLWAIGRDPKVWKNPEVFSPERFLESNIDYKGRDFELLPFGAGRRICPGLTLAYRMLNLMMANFLHSYDWKLEDGMHPKDLDMDEKFGITLQKVKPLQVIPVPR

SEQ ID NO: 15 - DNA, 1500 bp, Beta vulgaris
  source 1..1500: mol_type = other DNA
atggacaacactaccttggccttgatcttgtcatctttgttcgtttgcttccagttgatcaggtcctttattaaccatgccaagaagtctaacaaattgccaccaggtccaaagagaatgccaatttttggtaacatcttcgacttgggtgaaaagccacatagatcttttgctaacttggctaagattcacggtccattggtttctttacaattgggttctgttactaccgtcgttgtttcttctgctgatgttgctaaagaaatgttcttgaagaacgatcaagctttggccaacagaactattccagattctgttagagctggtgatcacgataagttgtctatgtcttggttgccagtttctgctaaatggcgtaacttgagaaagatttctgctgtccaactgttgtctacccaaagattggatgcttctcaagctcatagacaatctaaggttcaacagttgttggaatacgttcacgattgctctaaaaagggtcaaccagttgatattggtagagctgcttttactacctccttgaacttgttgtctaacaccttcttctctgttgaattggcctctcatgaatcttcagcttcccaagaattcaagcaattgatgtggaacatcatggaagaaatcggtagaccaaattacgccgattttttcccaattttgggttacttggatccattcggtatcagaagaagattggctggttacttcgaccaattgattgctgttttccaagacatcattggtgagagacaaaaaatcagatccgctaatttgtctggtggtaagcaaactaacgatatcttggataccctgctgaacttgtacgacgaaaaagaattatccatgggtgagatcaaccacttgttggttgatatttttgatgccggtactgataccactgcttctactttggaatgggctatggctgaattggttaagaatccagatatgatggttaaggtccaggacgaaattgaacaagctattggtaaaggttgctccatggttcaagaatctgatatctctaagttgccatacttgcaggccattatcaaagaaaccttgagattgcatccaccaaccgtttttttgttgccaagaaaagctgatgcagatgttgagttgtatggttacgttgttccaaagaatgctcaagtcttggttaacttgtgggctataggtagagatccaaaggtttggaaaaacccagaagttttctcaccagaaaggttcttggaatccaacattgattacaagggtagagactttgagttgttgccatttggtgctggtagaagaatttgtccaggtttgactttggcttacaggatgttgaatttgatgatggccaacttcttgcactcttacgattggaaattggaagatggtatgcaccctaaggatttggatatggacgaaaagtttggtatcaccttgcaaaaggttaagccattgcaagttattccagtgccaagatga

SEQ ID NO: 16 - protein, 499 aa, Beta vulgaris
MDNTTLALILSSLFVCFQLIRSFINHAKKSNKLPPGPKRMPIFGNIFDLGEKPHRSFANLAKIHGPLVSLQLGSVTTVVVSSADVAKEMFLKNDQALANRTIPDSVRAGDHDKLSMSWLPVSAKWRNLRKISAVQLLSTQRLDASQAHRQSKVQQLLEYVHDCSKKGQPVDIGRAAFTTSLNLLSNTFFSVELASHESSASQEFKQLMWNIMEEIGRPNYADFFPILGYLDPFGIRRRLAGYFDQLIAVFQDIIGERQKIRSANLSGGKQTNDILDTLLNLYDEKELSMGEINHLLVDIFDAGTDTTASTLEWAMAELVKNPDMMVKVQDEIEQAIGKGCSMVQESDISKLPYLQAIIKETLRLHPPTVFLLPRKADADVELYGYVVPKNAQVLVNLWAIGRDPKVWKNPEVFSPERFLESNIDYKGRDFELLPFGAGRRICPGLTLAYRMLNLMMANFLHSYDWKLEDGMHPKDLDMDEKFGITLQKVKPLQVIPVPR

SEQ ID NO: 17 - DNA, 1488 bp, Cleretum bellidiforme
  source 1..1488: mol_type = other DNA
atggactacaccaccttggtcatgattctgtctattgtgttcttctgctacaacctgttcaacttgttgttcactagaaagaacactaagttgccaccaggtccaaagactattccaattttcggtaacatcttcgagttgggtaaaaagccacatcaatcttttgctaacttggctaagattcacggtccattgatgtctttgaaattgggttctgttacgaccatcgttgtttcatctgctgaagttgctagagaaatgttcttgaagaacgaccagttgttgtccaatagaactgttccaaattctgttaccgctggtgatcatcataagactactatgtcttggttgccagtttctcaaaagtggcgtaacttcagaaagattaccgctgttcatttgttgtccccacaaagattggattcttgtcaagctttgagacaagctaaggttaagcagttgttcaactacatccatgaatgtgctcaaaagggtgaagctgttgatattggtaaagctgctttcaccacttccttgaacttgttgagcaatttgttcttctccgttgaattggccaaccacaaatcttcatcttcccaagaattcaagcagctgatctggaacattatggaagatatcggtaagccaaactacgctgattactttccagttttgaagtacgttgatccatccggtattagaagaagattggcttctaacttcaacaagctgatcgatgttttccagggtttcatcagattgagaatgtctaccaattcttcttgcggtgctactaatccaaatgatgttttggatgtcctgctgaacttgtacaaaggtgatgatttgaacatggacgagatcaaccatttgttggttgatattttcgatgccggtactgataccacttcttctacttttgaatgggctatggctgaattggttaagaacccaaaaatgatgaagaaggcccaagccgaaatccaacaagttttgggtaaagactccatcatcagagaatccgatattccaaatatgccatacttgcaggccatcatcaaagagactttgagattgcatccaccaaccgtttttttgttgccaagaaaagctgatgctgatgttgagttgtatggttacgttgttccaaagaacgcccaaatcttggttaatttgtgggctttgggtagagatcctttggtttggaaatctcctaatgtgttcaagccagaaaggttcttgggttccgaaattgatttcaagggaagagattttggcttgttgccatttggtgctggtagaagaatttgtccaggtatgaatttggcctacagaatgttgactttgatgttggctactctgttgcaatctttcgattggaaagttgctgatggtacaaacccacaagatatggatatggacgaaaagtttggtatcgccttgcaaaaaactaccccattgcaaattatccccgtctacaagtactga

SEQ ID NO: 18 - protein, 495 aa, Cleretum bellidiforme
MDYTTLVMILSIVFFCYNLFNLLFTRKNTKLPPGPKTIPIFGNIFELGKKPHQSFANLAKIHGPLMSLKLGSVTTIVVSSAEVAREMFLKNDQLLSNRTVPNSVTAGDHHKTTMSWLPVSQKWRNFRKITAVHLLSPQRLDSCQALRQAKVKQLFNYIHECAQKGEAVDIGKAAFTTSLNLLSNLFFSVELANHKSSSSQEFKQLIWNIMEDIGKPNYADYFPVLKYVDPSGIRRRLASNFNKLIDVFQGFIRLRMSTNSSCGATNPNDVLDVLLNLYKGDDLNMDEINHLLVDIFDAGTDTTSSTFEWAMAELVKNPKMMKKAQAEIQQVLGKDSIIRESDIPNMPYLQAIIKETLRLHPPTVFLLPRKADADVELYGYVVPKNAQILVNLWALGRDPLVWKSPNVFKPERFLGSEIDFKGRDFGLLPFGAGRRICPGMNLAYRMLTLMLATLLQSFDWKVADGTNPQDMDMDEKFGIALQKTTPLQIIPVYKY

SEQ ID NO: 19 - DNA, 1485 bp, Ercilla volubilis
  source 1..1485: mol_type = other DNA
atggatcataccaccttggccatgattttgtctgctattttcctgttgtacaatttggccaaggccatcttttctcattctaacacaaaattgccaccaggtccaaaaccagttccaatttttggtaacatcttcgagttgggtgaaaagccacatagatcttttgctaacttggctaagattcacggtccattgattactttgaagttgggttctgttacgaccatcgttgtttcatctgctgaagttgctaaagaaatgttcctgaagaacgatttgccattggctaatagaaacgttccaaactctgttactgctggtgatcatcataagttgactatgtcttggttgccagtttctccaaagtggaaaaccttcagaaagattaccgctgttcatttgttgtccccacaaagattggattcttgtcaagctttgagacacaccaaggttaagcaattgcatcaatacgttcaagaatgcgctaaaagaggtgaaccagttgatattggtaaggctgcttttactacctccttgaacttgttgtctaacctgttcttctctgttgaattggctaaccatacctcctcatcttcccaagaattcaaagaattgatctgggagatcatggaagatatcggtaagccaaattacgctgattacttcccaattttgaagtgcgttgatccatggggtattagaagaagattggcttctaacttcgacaagttgatcgaggtttttcagggtttcatcagaaagagattgtctaccggttccttttctgctattactccaaatgatgttctggacgtgttgctgaatctgttgaaagaaaaagaactgaacatgggcgaaatcaaccacttgttggttgatatttttgatgccggtactgataccacttcttctacatttgaatgggctatggctgagttggtcagaaatcaagaaatgatgaagaaggcccaagacgaaatcgaacaagttttgggtaaagatgccatcatccaagaatccgatattccaaaaatgccatacttgcaggccattatcaaagaaaccttgagattgcatccaccaaccgtttttttgttgccaagaaaggctacttctaacgttgagttgtatggttacgttgttccaaagaacgcccaaatcttggttaatttgtgggctattggtagagatccaaaggtttgggataatccaaacatgttctctccagaaaggttcttgaactccgaaattgatgttaagggtagagactttggtttgttgccttttggtgctggtagaagaatttgtccaggtatgaatttggcctacagaatgttgactttgatgttggctactctgttgcaatctttcgattggaaattaggtgatggtgttaacccaaaggatttggacatggaagaaaagtttggtatcgccttgcaaaagactaagccattgcaagttattcccgtcttgaagtactga

SEQ ID NO: 20 - protein, 494 aa, Ercilla volubilis
MDHTTLAMILSAIFLLYNLAKAIFSHSNTKLPPGPKPVPIFGNIFELGEKPHRSFANLAKIHGPLITLKLGSVTTIVVSSAEVAKEMFLKNDLPLANRNVPNSVTAGDHHKLTMSWLPVSPKWKTFRKITAVHLLSPQRLDSCQALRHTKVKQLHQYVQECAKRGEPVDIGKAAFTTSLNLLSNLFFSVELANHTSSSSQEFKELIWEIMEDIGKPNYADYFPILKCVDPWGIRRRLASNFDKLIEVFQGFIRKRLSTGSFSAITPNDVLDVLLNLLKEKELNMGEINHLLVDIFDAGTDTTSSTFEWAMAELVRNQEMMKKAQDEIEQVLGKDAIIQESDIPKMPYLQAIIKETLRLHPPTVFLLPRKATSNVELYGYVVPKNAQILVNLWAIGRDPKVWDNPNMFSPERFLNSEIDVKGRDFGLLPFGAGRRICPGMNLAYRMLTLMLATLLQSFDWKLGDGVNPKDLDMEEKFGIALQKTKPLQVIPVLKY

SEQ ID NO: 21 - DNA, 1494 bp, Phytolacca dioica
  source 1..1494: mol_type = other DNA
atggatcataccaccttggccatgattttgtctgctattttcctgctgtacaacttggttaagttggccattttctctcagtctaacacaaaattgccaccaggtccaaaaccattgccaatttttggtaacatcttcgagttgggtgataagccacatagatcttttgctaacttggctaagattcacggtccattgattactttgaagttgggttctatcacgaccatcgttgtttcatctgctgaagttgctaaagaaatgttcttgaagaacgatcaaccactggctaatagaaacgttccaaattctgttactgctggtgatcatcataagttgactatgtcttggttgccagtttctccaaagtggaaaaccttcagaaagattaccgctgttcatttgttgtccccacaaagattggatgcttgtcaagctttgagacataccaacgttaagcaattgcacgaatacgttcaagaatgtgctcaaagaggtcaaccagttgatattggtaaagctgctttcaccacttccttgaacttgttgtctaacttgttcttctccgttgaattggctaaccatacctcttctaactcccaagaattcaaagaattgatctgggacatcatggaagatatcggtaagccaaattacgctgattacttcccagttttgaagtgtgttgatccatggggtattagaagaaggttggaatctaacttcgacaagttgatcgaggtctttcaaggtttcatcagaaagagattgtctaccggttctttctctgcttctgaaactactccaaatgatgttttggacgtcctgctgaatctgttcaaagaaaaagaactgaacatgggcgagatcaaccatttgttggttgatatttttgatgccggtactgataccacttcttctacttttgaatgggctatggctgaattggttagaaacccagatatgatgaagaaggcccaagacgaaattgaacaagttttgggtagagatgccatcatccaagaatctgatattccaaagatgccatacttgcaggccattatcaaagaaactttgagattgcatccaccaaccgtttttttgttgccaagaaaggctactaccaatgttgacttgtatggttacgttgttccaaagaacgcccaaatcttggttaatttgtgggctattggtagagatccaactgtttgggataatccaaatatgttctctccagagaggttcttgaactccgatattgatgtaaagggtagagactttggtttgttgccttttggtgctggtagaagaatttgtccaggtatgaatttggcctacagaatgttgacattgatgttggctactctgttgcaatctttcaactggaaattaggtgatggtgttaacccaaaggatttggatatggacgaaaagtttggtatcgccttgcaaaaaactaagccattgcaagtcattcccgtgttcaagtactaa

SEQ ID NO: 22 - protein, 497 aa, Phytolacca dioica
MDHTTLAMILSAIFLLYNLVKLAIFSQSNTKLPPGPKPLPIFGNIFELGDKPHRSFANLAKIHGPLITLKLGSITTIVVSSAEVAKEMFLKNDQPLANRNVPNSVTAGDHHKLTMSWLPVSPKWKTFRKITAVHLLSPQRLDACQALRHTNVKQLHEYVQECAQRGQPVDIGKAAFTTSLNLLSNLFFSVELANHTSSNSQEFKELIWDIMEDIGKPNYADYFPVLKCVDPWGIRRRLESNFDKLIEVFQGFIRKRLSTGSFSASETTPNDVLDVLLNLFKEKELNMGEINHLLVDIFDAGTDTTSSTFEWAMAELVRNPDMMKKAQDEIEQVLGRDAIIQESDIPKMPYLQAIIKETLRLHPPTVFLLPRKATTNVDLYGYVVPKNAQILVNLWAIGRDPTVWDNPNMFSPERFLNSDIDVKGRDFGLLPFGAGRRICPGMNLAYRMLTLMLATLLQSFNWKLGDGVNPKDLDMDEKFGIALQKTKPLQVIPVFKY

SEQ ID NO: 23 - DNA, 1512 bp, Acleisanthes obtusa
  source 1..1512: mol_type = other DNA
atggaaaacaccaccttgggtgttattctggccattattttcttgaccttccacgtcatgaagctgttgttgtcatctaacaaagctaaattgccaccaggtccaaaaccattgccaattattggtaacatcttggagttgggtgataagccacatagatcttttgctaacttggctaagattcacggtccattgattactttgaagttgggttctgttacgactatcgttgtctcttcatctgaagttgccaaagaaatgttcttgaagaacgatcaagctttggccaacagaactattccagattctgttagagctggtaaccacgataagttgtctatgtcttggttgccagtttctccaaaatggcgtaacttgagaaagatttctgccgttcaactgctgtcatctcaaagattggatgcttctcaagctcacagacaagttaaggttgaacagttgatcgaatacgtcaaagagtgctctaagattggtcaatgcgttgatattggtagagttgctttcaccacttccttgaacttgttgtctaacaccttcttcagcaaagaattggcctctttcgattctaacaacgcccaagaattcaagcaattgatgtggtgcattatggaagaaatcggtagaccaaattacgccgattactttccaattttgggttacgttgatccattcggtgttagaagaagattgtccagatacttcgaccagttgattgaagtgttccaacagatcattagagagaggttgtctaaggataacaagatcgtcgataacaacaacgatgttttggctactttgctgcacttgtacaaacagaacgaattgtctatggacgagatcaaccatttgttggttgatattttcgatgccggtactgatacaactgcttctactttggaatgggctatgtccgaattgattaagaacccacatattatgattaaggcccaagccgaagttagacaagctacttcttctagaggtggtgctactgttgttgatgttcaagaatccgatatcaacaacttgccttacattcaggccatcatcaaagaatccttgagattgcatccaccaaccgtttttttgttgccaagaaaagctgatgttgacgttcagttgtacggttatttggttccaaagaatgcccaagttttggttaacttgtgggctataggtagagatccaaatgtttggccagatccagaagtttttagaccagaaagattcttggattgcgaagttgatgttaagggtagagattttgagttgttgccatttggtgctggtagaagaatttgtcctggtttgtctttggcttacaggatgttgaatctgatgttggctaatctggttcattccttcgattggaaattgccaaacgtcgaaaacaagtctggttctgatccagatgaattggatatggacgaaaagttcggtatcaccttgcaaaaagttaagccattgcaaatcatccccatgtccagatga

SEQ ID NO: 24 - protein, 503 aa, Acleisanthes obtusa
MENTTLGVILAIIFLTFHVMKLLLSSNKAKLPPGPKPLPIIGNILELGDKPHRSFANLAKIHGPLITLKLGSVTTIVVSSSEVAKEMFLKNDQALANRTIPDSVRAGNHDKLSMSWLPVSPKWRNLRKISAVQLLSSQRLDASQAHRQVKVEQLIEYVKECSKIGQCVDIGRVAFTTSLNLLSNTFFSKELASFDSNNAQEFKQLMWCIMEEIGRPNYADYFPILGYVDPFGVRRRLSRYFDQLIEVFQQIIRERLSKDNKIVDNNNDVLATLLHLYKQNELSMDEINHLLVDIFDAGTDTTASTLEWAMSELIKNPHIMIKAQAEVRQATSSRGGATVVDVQESDINNLPYIQAIIKESLRLHPPTVFLLPRKADVDVQLYGYLVPKNAQVLVNLWAIGRDPNVWPDPEVFRPERFLDCEVDVKGRDFELLPFGAGRRICPGLSLAYRMLNLMLANLVHSFDWKLPNVENKSGSDPDELDMDEKFGITLQKVKPLQIIPMSR

SEQ ID NO: 25 - DNA, 1512 bp, Mirabilis multiflora
  source 1..1512: mol_type = other DNA
atggaaaacaccatgttgggtgttattctggctactattttcttgaccttccacatcatgaagatgttcttgtctccatctaaagctaaattgccaccaggtccaagaccattgccaattattggtaacattttggagttgggtgataagccacatagatcttttgctaacttggctaagattcacggtccattggttactttgaaattgggttctgttacgactatcgtcgtgtcatcttctgaagttgctaaagaaatgttcctgaagaacgatcaaccattggccaacagaactattccagattctgttagagctggtaaccacgataagttgtctatgtcttggttgccagtttcaccaaaatggcgtaacttgagaaagatttctgctgtccaactgttgtctacccaaagattggatgcttctcaagctcatagacaagccaagattcaacaattgatcgagtacatcaagaagtgctccaagattggtcaatacgttgatattggtcaagttgccttcaccacttctttgaacttgttgtctaacaccttcttcagcaaagaattggcctctttcgattctaacaacgcccaagagtttaagcaattgatgtggtgcattatggaagaaatcggtagaccaaattacgccgattactttcctattttgggttacgttgatccattcggtgctagaagaagattgtctagatacttcgaccagttgatcgaagttttccaagtcatcattagagaaaggttgacccacgataacaacatcgttggtaacaacaacgatattttggctaccttgctggacttgtacaaacagaatgaattgtccatggatgagatcaaccacttgttggttgatattttcgatgctggtactgataccactgcttctactttggaatgggctatgtctgaattgatcaagaacccacatattatggtcaaggcccaagaagaagttagacaagctactatgtctagaggtggtgctactgttgctgaaattcaagaatccgatatcaacaacttgccctacattcagtccattgtcaaagaaaccttgagattgcatccaccaactgtttttttgttgccaagaaaggctgaagttgatgttcagttgttcggttatgttgttccaaagaacgctcaagttttggttaacttgtgggctattggtagagatccaaatgtttggccagatccagaagtttttaagccagaaagattcatggattgcgaaatcgatgttaagggtagagattttgagttgttgccatttggtgctggtagaagaatttgtccaggtttgtctttggcttacaggatgttgaatttgatgttggctaacatggtccattccttcgaatggaaattgccaggtgttgaaaatggtttcggttccgaaatggattctttggatatggacgaaaagttcggtatcaccttgcaaaaagttcagccattgaaggttatcccagtctctagataa

SEQ ID NO: 26 - protein, 503 aa, Mirabilis multiflora
MENTMLGVILATIFLTFHIMKMFLSPSKAKLPPGPRPLPIIGNILELGDKPHRSFANLAKIHGPLVTLKLGSVTTIVVSSSEVAKEMFLKNDQPLANRTIPDSVRAGNHDKLSMSWLPVSPKWRNLRKISAVQLLSTQRLDASQAHRQAKIQQLIEYIKKCSKIGQYVDIGQVAFTTSLNLLSNTFFSKELASFDSNNAQEFKQLMWCIMEEIGRPNYADYFPILGYVDPFGARRRLSRYFDQLIEVFQVIIRERLTHDNNIVGNNNDILATLLDLYKQNELSMDEINHLLVDIFDAGTDTTASTLEWAMSELIKNPHIMVKAQEEVRQATMSRGGATVAEIQESDINNLPYIQSIVKETLRLHPPTVFLLPRKAEVDVQLFGYVVPKNAQVLVNLWAIGRDPNVWPDPEVFKPERFMDCEIDVKGRDFELLPFGAGRRICPGLSLAYRMLNLMLANMVHSFEWKLPGVENGFGSEMDSLDMDEKFGITLQKVQPLKVIPVSR

SEQ ID NO: 27 - DNA, 1485 bp, Acleisanthes obtusa
  source 1..1485: mol_type = other DNA
atggaccaaactaccttggccatgttgttgtctgcattatacctgctgtacaacctgtacaaggttatcttcactcagtccaattctaaattgccaccaggtccaaaaccattgccaattttgggtaacattttcgaggttggtaacaagccacatagatcttttgctaacttggctaagattcacggtccattgattactttgaagttgggttctgttacgaccatcgttgtttcttcagctaaagttgccgaagagatgttcttgaagaatgatttgccattggccaacagaaacgttccaaattctgttactgctggtgatcatcataagttgactatgtcttggttgccagtttctccaaagtggaaaaccttcagaaagattaccgctgttcatttgttgtccccacaaagattggatgcttgtcaagctttgagacatgctaaggttaagcaattgcacgaatacgttcaagattgtgctaaaaagggtcaagccgttgatattggtaaagctgcttttactacctccttgaacttgctgtctaacttgttcttctctgttgaattggctcaacacacctcttcatcttcccaacatttcaaagaattgatctgggacatcatggaagatatcggtaagccaaattacgctgattatttcccagctttgaagtgtgttgatccatggggtattagaagaagattggctgctaatttcgaaaggttgatcgatgttttccagggtttcattagacagaggttgtccattaactcttctactgttacttctgcctctgatgttttggatgtcttgctgaacttgtacaaagaaaaagaactgaacatgggcgagatcaaccatttgttggttgatatttttgatgccggtactgataccacttcttctacatttgaatgggctatggctgaattggttagacatccagaaattatgaagaaggcccaagacgaaatcgaacaagttttaggtaaggatgccattatccaagaagccgatattccaaaaatgccatacttgcaagccatcatcaaagaaaccttgagattgcatccaccaaccgtttttttgttgccaagaaaggctactaccaacgtcgaattatatggttacgttgttccaaagaacgcccaaatcttggttaatttgtgggctattggtagagatccattggtttgggataatccaaacaagttctctccagaaaggttcttgaactccgatattgatgttaagggtagagactttggtttgttgccttttggtgctggtagaagaatttgtccaggtatgaatttggcctacagaatcttgactttgatgttggcaactctgttgcaatctttcgaatggatggttgaaaatggtgaaaaccctgaagatttggacatggacgaaaaatttggtatcgccttgcaaaagactaagccattggaaattatcccagtcatcaagcactga

SEQ ID NO: 28 - protein, 434 aa, Acleisanthes obtusa
IHGPLITLKLGSVTTIVVSSAKVAEEMFLKNDLPLANRNVPNSVTAGDHHKLTMSWLPVSPKWKTFRKITAVHLLSPQRLDACQALRHAKVKQLHEYVQDCAKKGQAVDIGKAAFTTSLNLLSNLFFSVELAQHTSSSSQHFKELIWDIMEDIGKPNYADYFPALKCVDPWGIRRRLAANFERLIDVFQGFIRQRLSINSSTVTSASDVLDVLLNLYKEKELNMGEINHLLVDIFDAGTDTTSSTFEWAMAELVRHPEIMKKAQDEIEQVLGKDAIIQEADIPKMPYLQAIIKETLRLHPPTVFLLPRKATTNVELYGYVVPKNAQILVNLWAIGRDPLVWDNPNKFSPERFLNSDIDVKGRDFGLLPFGAGRRICPGMNLAYRILTLMLATLLQSFEWMVENGENPEDLDMDEKFGIALQKTKPLEIIPVIKH

SEQ ID NO: 29 - DNA, 1488 bp, Abronia nealleyi
  source 1..1488: mol_type = other DNA
atggactacttgaccatcgtcatgttggtgtctatcgttttcttcctgtactccttgttgaagatgatgttcatcactcattccaacgctcaattgccaccaggtccaaaacctatgccattgattggtaacattttggagattggtgaaaagccacatagatcctttgctaacttggctaaatctcatggtccattgatgtctttgagattgggtagagttactaccatcgttgtttcatctgctgaagttgccaaagaaatgttcttgaagaacgaccaatctttgtgcgatagatgtgttccaaattctgttactgctggtgatcatcataagttgactatgtcttggttgccagtttcaccaaagtggaagaacttcagaaagattaccgctgttcatttgttgtctccacaaagactagatgcttgtcatgctttgagacatgctaaagttaagcagttgtacgagtacattcaagaatgcgctattaagggtgaagccgttgatattggtaaagctgcttttactacctccttgaacttgctgtctaacttgttcttctctgttgaattggctcatcatacctctaacacctctcaagaattcaagcaattgatctgggacatcatggaagatatcggtaagccaaattacgctgattacttcccactgttgaaatacgttgatccattgggtatcagacatagattggctgctaatttcgataagctgatcgacgttttccagtcctttattagaaagaggctgctgtcctcttattcttctgctacttctttgaacgatgtcttggacgttttgttgaagctgtacaaagaaaaggctttgaacatgggtgaaatcaaccacttgttggttgatattttcgatgctggtactgataccacctctaatacttttgaatgggctatggccgaattgatcagacatccaattatgatgagaagggcccaaactgaaattgctttggttttgggtaaagacccaaccatcaaagaagctgacgttgctaatatgccatacttgcaagccattatcaaagaaaccttgagattgcatccaccaaccgtttttttgttgccaagaaaggctattaccgacgttaagttgtatggttacgttgtcccaaagaactcccaaatcttggttaatttgtgggccattggtagagatccaaaggtttggaacaatccaaacgaattcatgccagacagattcttgaactccgatattgatgttaagggtagagactttggtttgttgccttttggtgctggtagaagaatttgtccaggtatgaatttggcctacagaatgttgactttgatgttggctactctgttgcaatctttcgattggaaattgccacatggtattacccctaaggatttggatatggacgaaaagttcggtatctccttgcaaaaaactaagccattgcagttgatccccatcttgaagtactaa

SEQ ID NO: 30 - protein, 495 aa, Abronia nealleyi
MDYLTIVMLVSIVFFLYSLLKMMFITHSNAQLPPGPKPMPLIGNILEIGEKPHRSFANLAKSHGPLMSLRLGRVTTIVVSSAEVAKEMFLKNDQSLCDRCVPNSVTAGDHHKLTMSWLPVSPKWKNFRKITAVHLLSPQRLDACHALRHAKVKQLYEYIQECAIKGEAVDIGKAAFTTSLNLLSNLFFSVELAHHTSNTSQEFKQLIWDIMEDIGKPNYADYFPLLKYVDPLGIRHRLAANFDKLIDVFQSFIRKRLLSSYSSATSLNDVLDVLLKLYKEKALNMGEINHLLVDIFDAGTDTTSNTFEWAMAELIRHPIMMRRAQTEIALVLGKDPTIKEADVANMPYLQAIIKETLRLHPPTVFLLPRKAITDVKLYGYVVPKNSQILVNLWAIGRDPKVWNNPNEFMPDRFLNSDIDVKGRDFGLLPFGAGRRICPGMNLAYRMLTLMLATLLQSFDWKLPHGITPKDLDMDEKFGISLQKTKPLQLIPILKY

SEQ ID NO: 31 - DNA, 1491 bp, Phytolacca Americana
  source 1..1491: mol_type = other DNA
atggatcataccaccttggccatgatcttgtctgttattttcctgctgtacaacttggttaaggccatcttttctcaatccaacacaaaattgccaccaggtccaaaaccagttccaatttttggtaacatcttcgagttgggtgataagccacatagatcttttgctaacttggctaagattcacggtccattgattactttgaagttgggttctgttacgaccatcgttgtttcatctgctgaagttgctaaagaaatgttcttgaccaacgatcagttgctggctaatagaaacgttccaaattctgttactgctggtgatcatcataagttgactatgtcttggttgccagtttctccaaagtggaaaaccttcagaaagattaccgctgttcatttgttgtccccacaaagattggatgcttgtcaagctttgagacataccaaagttaagcagttgtacgaatacgttcaagaatgtgctaaaagaggtgaagccgttgatattggtaaagctgcttttactacctccttgaacttgctgtctaacttgttcttctctgttgaattggctaaccacacctcttcatcttcccaagaattcaaagaattgatctgggacatcatggaagatatcggtaagccaaattacgctgattacttcccagttttgaagtgtgttgatccatggggtattagaagaaggttggaatctaacttcgacaagttgatcgaggtgttccagtctttcattagaaagagattgtctaccgaaccattttctgcctctgctaaaactccaaatgatgttttggacgtgctgctgaacttgttgaaagaagaggaattgaacatgggtgagatcaaccatttgttggttgatattttcgatgccggtactgataccacttcttctacatttgaatgggctatggctgaattggttagaaacccagaaatgatgaagaaggcccaagacgaaattgaacaagttttgggtaaagacgccatcatccaagaatctgatattccaaaaatgccatacttgcaggccattatcaaagaaaccttgagattgcatccaccaaccgtttttttgttgccaagaaaagcctcttctaacgttgagttgtatggttacgttgttccaaagaacgcccaaatcttggttaatttgtgggctattggtagagatccaactgtttgggataatccaaatatgttctctccagagaggttcttgaactccgatattgatgttaagggtagagactttggtttgttgccttttggtgctggtagaagaatttgtccaggtatgaatttggcctacagaatgttgactttgatgttggctactctgttgcaatctttcgattggaaattaggtgatggtgttaacccaaaggatttggatatggacgaaaagtttggtatcgccttgcaaaaaactaagccattgcaagtcattcccgtcttgaagtactaa

SEQ ID NO: 32 - protein, 496 aa, Phytolacca Americana
MDHTTLAMILSVIFLLYNLVKAIFSQSNTKLPPGPKPVPIFGNIFELGDKPHRSFANLAKIHGPLITLKLGSVTTIVVSSAEVAKEMFLTNDQLLANRNVPNSVTAGDHHKLTMSWLPVSPKWKTFRKITAVHLLSPQRLDACQALRHTKVKQLYEYVQECAKRGEAVDIGKAAFTTSLNLLSNLFFSVELANHTSSSSQEFKELIWDIMEDIGKPNYADYFPVLKCVDPWGIRRRLESNFDKLIEVFQSFIRKRLSTEPFSASAKTPNDVLDVLLNLLKEEELNMGEINHLLVDIFDAGTDTTSSTFEWAMAELVRNPEMMKKAQDEIEQVLGKDAIIQESDIPKMPYLQAIIKETLRLHPPTVFLLPRKASSNVELYGYVVPKNAQILVNLWAIGRDPTVWDNPNMFSPERFLNSDIDVKGRDFGLLPFGAGRRICPGMNLAYRMLTLMLATLLQSFDWKLGDGVNPKDLDMDEKFGIALQKTKPLQVIPVLKY

SEQ ID NO: 33 - DNA, 1500 bp, Chenopodium quinoa
  source 1..1500: mol_type = other DNA
atggaacataccaccttggccttgatcttgtccattttgttcatttgcttccacttggtcaggtcctttgtttctcattctactaagtctaacaagttgccaccaggtccaaaaagaatgccaattttcggtaacatcttggacttgggtgaaaagccacatagatcttttgctaacttggctaagattcacggtccattggtttctttacaattgggttctatcacgaccatcatcgtttcttcagctgatgttgctaaagaaatgttcttgaagaacgatcagttgttggctaacagaaccattccagattctgttagagctggtaaccatgacaaattgtctatgtcttggttgccaatttctgctaagtggcgtaacttgagaaagatttccgctgtccaactgttgtccaatcaaagattggatgcttctcaagctcatagacaagctaaagttgaacaactgttggcttacgttcaagactgttctaaaaaaggtcaaccagttgatattggtagagctgcttttactacctccttgaacttgttgtctaacaccttcttctctaccgaattggcttctcatgaatccaacaactctcaagaattcaagcagttgatgtggaacatcatggaagaaattggtagaccaaactacgctgattacttcccaattttgggttacgttgatccattcggcattagaagaagattggctgcatacttcgataagttgattgctgttttccaagacatcatctgcgaaagacaaaagatcaggtctacgaaggtttcctctgaaaaacaaaccggtgatatcttggataccctgttgaacttatacgacgaaaacgaattatccatgggtgagatcaaccacttgttggttgatatttttgatgccggtactgataccactgcttctactttggaatgggctatggctgaattggttaagaatccaggtatgatgatcagagtccagaacgaaattgaattggctattggtaagggttgctccatggttcaagaagctgatatttctaagttgccatacttgcaggccatcatcaaagaaactttgagattgcatccaccaaccgtttttttgttgccaagaaaggctgatatcgatgttgagttgtacggttatgttgttccaaagaacgctcaagttttggttaacttgtgggctataggtagagatccaaaggtttggaaaaacccagaaatcttctcaccagaaagattcttgggttgcgatattgatttcaagggtagagattttgagttgttgccatttggtgctggtagaagaatttgtccaggtttgactttggcttacaggatgttgaatctaatgatggccaacttcgttcactctttcgattggaaattggaggatggtatgaaccctaaggatttggatatggacgaaaagttcggtatcaccttgcaaaaagttaagccattgcaagttatcccagtccacagataa

SEQ ID NO: 34 - protein, 499 aa, Chenopodium quinoa
MEHTTLALILSILFICFHLVRSFVSHSTKSNKLPPGPKRMPIFGNILDLGEKPHRSFANLAKIHGPLVSLQLGSITTIIVSSADVAKEMFLKNDQLLANRTIPDSVRAGNHDKLSMSWLPISAKWRNLRKISAVQLLSNQRLDASQAHRQAKVEQLLAYVQDCSKKGQPVDIGRAAFTTSLNLLSNTFFSTELASHESNNSQEFKQLMWNIMEEIGRPNYADYFPILGYVDPFGIRRRLAAYFDKLIAVFQDIICERQKIRSTKVSSEKQTGDILDTLLNLYDENELSMGEINHLLVDIFDAGTDTTASTLEWAMAELVKNPGMMIRVQNEIELAIGKGCSMVQEADISKLPYLQAIIKETLRLHPPTVFLLPRKADIDVELYGYVVPKNAQVLVNLWAIGRDPKVWKNPEIFSPERFLGCDIDFKGRDFELLPFGAGRRICPGLTLAYRMLNLMMANFVHSFDWKLEDGMNPKDLDMDEKFGITLQKVKPLQVIPVHR

SEQ ID NO: 35 - DNA, 1485 bp, Mirabilis multiflora
  source 1..1485: mol_type = other DNA
atggaccaaactaccttggccatgttgttgtctgcattatacctgctgtacaacttgttcaaggttatcttcacccagtccaattctaaattgccaccaggtccaaaaccattgccaattttgggtaacattttcgagttgggtgataagccacataggtcttttaacaacttggctaagattcacggtccattgattactttgaagttgggttctgttacgaccatcgttgtttcttcagctaaagttgccgaagagatgttcttgaagaatgatttgccattggccaacagaaacgttccaaattctgttactgctggtgatcatcataagttgactatgtcttggttgccagtttctccaaagtggaaaaccttcagaaagattaccgctgttcatttgttgtccccacaaagattggatgcttgtcaagctttgagacatgctaaagttaagcagttgtaccaatacgttcaagattgtgctaaaaagggtgaagccgttgatattggtaaagctgcctttactacttccctgaacttgttgtctaacctgttcttctctgttgaattggctcaacatacctcctcttcatcccaacatttcaaagaattgatctgggacatcatggaagatatcggtaagccaaattacgctgattatttcccagctttgaagtgtgttgatccatggggtattagaagaagattggctgctaatttcgaaaggttgatccaagtgttccagaacttcattagacagagattgtctaccgatccatcttctgttacaaatgcttctgatgttttggacgtcctgctgaacctatacaaagaaaaagaactgaacatgggcgagatcaaccatttgttggttgatatttttgatgccggtactgataccacttcttctacatttgaatgggctatggctgaattggttagacatccagaaattatgaagaaggcccaagacgaaatcgaacaagttttaggtaaggatgccaccattcaagaagctgatattccaaaaatgccatacttgcaggccatcatcaaagaaactttgagattgcatccaccaaccgtttttttgttgccaagaaaggctactaccaacgtcgaattatatggttacgttgttccaaagaacgcccaaatcttggttaatttgtgggctattggtagagatccattggtttgggatcagccaaacaaattttctccagaaaggttcttgaactccgatatcgatgttaagggtagagattttggtttgttgccttttggtgctggtagaagaatttgtccaggtatgaatttggcctacagaatgttgactttgatgttggctactctgttgcaatctttcgaatggaaggttcaaaatggtgaaaagcctgaagatttggacatggacgaaaaatttggtatcgccttgcaaaagactaagccattggaaattattcccgtcctgaagtactga

SEQ ID NO: 36 - protein, 494 aa, Mirabilis multiflora
MDQTTLAMLLSALYLLYNLFKVIFTQSNSKLPPGPKPLPILGNIFELGDKPHRSFNNLAKIHGPLITLKLGSVTTIVVSSAKVAEEMFLKNDLPLANRNVPNSVTAGDHHKLTMSWLPVSPKWKTFRKITAVHLLSPQRLDACQALRHAKVKQLYQYVQDCAKKGEAVDIGKAAFTTSLNLLSNLFFSVELAQHTSSSSQHFKELIWDIMEDIGKPNYADYFPALKCVDPWGIRRRLAANFERLIQVFQNFIRQRLSTDPSSVTNASDVLDVLLNLYKEKELNMGEINHLLVDIFDAGTDTTSSTFEWAMAELVRHPEIMKKAQDEIEQVLGKDATIQEADIPKMPYLQAIIKETLRLHPPTVFLLPRKATTNVELYGYVVPKNAQILVNLWAIGRDPLVWDQPNKFSPERFLNSDIDVKGRDFGLLPFGAGRRICPGMNLAYRMLTLMLATLLQSFEWKVQNGEKPEDLDMDEKFGIALQKTKPLEIIPVLKY

SEQ ID NO: 37 - DNA, 1500 bp, Chenopodium quinoa
  source 1..1500: mol_type = other DNA
atggaacataccatcctggccttgatcttgtccattttgttcatttgcttccacttggtcaggtcctttgtttctcattctactaagtctaacaagttgccaccaggtccaaaaagaatgccaattttcggtaacatcttcgacttgggtgaaaagccacatagatcttttgctaacttggctaagattcacggtccattggtttctttacaattgggttctatcacgaccatcgttgtttcttcagctgatgttgctaaagaaatgttcttgaagaacgatcaagctttggccaacagaactattccagattctgttagagctggtgatcacgataagttgtctatgtcttggttgccaatttctgctaagtggcgtaacttgagaaagatttccgctgtccaactgttgtccaatcaaagattggatgcttctcaagctcatagacaagctaaagttgaacagttgttggcttacgttcaagactgttctaaaaagggtcaaccagttgatattggtagagctgcttttactacctccttgaacttgttgtctaacaccttcttctctaccgaattggcttctcatgaatccaacaactctcaagaattcaagcagttgatgtggaacatcatggaagaaattggtaagccaaactacgctgatttcttcccaattttgggttacgttgatccattcggcattagaagaagattggctggttacttcgacaagttgattgctgttttccaagacatcatttgcgagagacaaaagattaggtctaccaaggtttcctctgaaaagcaaactggtgatatcttggataccctgttgaacttatacgacgaaaacgaattgtccatgggtgaaatcaaccacttgttggttgatattttcgatgctggtactgataccactgcttctactttggaatgggctatggctgaattggttaagaatccaggtatgatgatcagagtccagaacgaaattgaattggctattggtaagggttgctccatggttcaagaatctgatatttctaagctgccatacttgcaggccattatcaaagaaactttgagattgcatccaccaaccgtttttttgttgccaagaaaagcagatgttgacgttgagttgtacggttatgttgttccaaagaatgctcaagtcttggttaacttgtgggctataggtagagatccaaaggtttggaaaaacccagaaatctttagcccagaaagattcattggttgcgatatcgatttcaagggtagagatttcgagttgttgccatttggtgctggtagaagaatttgtccaggtttgactttggcatacaggatgttgaatttgatgatggcttacttcgttcactccttcgattggaaattggaagatggtatgaacccaaaggacttggatatggacgaaaagtttggtatcaccttgcaaaaggttaagccattgcaagttatcccagtccaaagatga

SEQ ID NO: 38 - protein, 499 aa, Chenopodium quinoa
MEHTILALILSILFICFHLVRSFVSHSTKSNKLPPGPKRMPIFGNIFDLGEKPHRSFANLAKIHGPLVSLQLGSITTIVVSSADVAKEMFLKNDQALANRTIPDSVRAGDHDKLSMSWLPISAKWRNLRKISAVQLLSNQRLDASQAHRQAKVEQLLAYVQDCSKKGQPVDIGRAAFTTSLNLLSNTFFSTELASHESNNSQEFKQLMWNIMEEIGKPNYADFFPILGYVDPFGIRRRLAGYFDKLIAVFQDIICERQKIRSTKVSSEKQTGDILDTLLNLYDENELSMGEINHLLVDIFDAGTDTTASTLEWAMAELVKNPGMMIRVQNEIELAIGKGCSMVQESDISKLPYLQAIIKETLRLHPPTVFLLPRKADVDVELYGYVVPKNAQVLVNLWAIGRDPKVWKNPEIFSPERFIGCDIDFKGRDFELLPFGAGRRICPGLTLAYRMLNLMMAYFVHSFDWKLEDGMNPKDLDMDEKFGITLQKVKPLQVIPVQR

SEQ ID NO: 39 - DNA, 1503 bp, Phytolacca Americana
  source 1..1503: mol_type = other DNA
atggaccataccaccattgccatgttggtttctatcgttttcttgtgcttccacctgatcaagtctttctcatctcattctaagaacccatctcaattgccaccaggtccaaaaccattaccaattttcggtaacatcttcgagttgggtgaaaagccacatagatcttttgctagattggccaaaattcacggtccattgatttctttgaagttgggttctgttacgactatcgttgtctcttcatctgaagttgccaaagaaatgttcttgaagcacgatcaagttttggctaacagaactatcccagattctgttagagctggtaaccatgataagttgtctatgtcttggttgccagtttctgttaagtggcgttctttgagaaagatcgttgttgttcagttgttctctacccaaagattggatgtctctcaatctttgagacatgccaaggttcaacaattgcatgagtacattaccgagtgttctaaaaagggtgaaccagttgatattggtagagctgcttttactacctccttgaacttgttgtctaacaccttcttctctatggaattggccaaccattcttcatcagcttctcaagaattcaagcaattgatgtggtgcatcatggaagaaattggtagaccaaattacgccgatttcttcccaattttgggttacgttgatccattcggtatcagaagaagattggctgtttacttcgataagttgatcgctgtgttccaagaaatcatcagagaaagacaaaaggccagattcactaacttgtctactaccaacgatgttttggacaccttgctgaacttgtaccaagaaaatgaattgagcatggacgagatcaaccacttgttggttgatatttttgatgccggtactgataccactgcttctacaatggaatgggctatggctgaattggttaagaatccagatattatgttgaaggccaggatcgaaatcaaacaagctttgggtaatgactccagcttgatcattcaagaatctgatattgccaagctgccatacttgcaagctattgtcaaagaaaccttgagattgcatccaccaaccgtttttttgttgccaagaaaagctgaagctaacgtccaattatacggctacttgattccaaagaatgcccagttgttggttaacttgtgggctataggtagagatccaaatgtttggaatgaccccttggttttttcaccagaaagattcttggcttcctcctcttgtgaaattgatgttaagggtagacactttgagttgttgccatttggtgctggtagaagaatttgtccaggtttgactttggcatacaggatgttgaatttgatgttggtgactctggttaactccttcgattggaaattggaagatgtcacttctgccaaggatttggatatggacgaaaagtttggtatcaccttgcaaaaggttaagccattgcaattgatccccattccaaagtga

SEQ ID NO: 40 - protein, 500 aa, Phytolacca Americana
MDHTTIAMLVSIVFLCFHLIKSFSSHSKNPSQLPPGPKPLPIFGNIFELGEKPHRSFARLAKIHGPLISLKLGSVTTIVVSSSEVAKEMFLKHDQVLANRTIPDSVRAGNHDKLSMSWLPVSVKWRSLRKIVVVQLFSTQRLDVSQSLRHAKVQQLHEYITECSKKGEPVDIGRAAFTTSLNLLSNTFFSMELANHSSSASQEFKQLMWCIMEEIGRPNYADFFPILGYVDPFGIRRRLAVYFDKLIAVFQEIIRERQKARFTNLSTTNDVLDTLLNLYQENELSMDEINHLLVDIFDAGTDTTASTMEWAMAELVKNPDIMLKARIEIKQALGNDSSLIIQESDIAKLPYLQAIVKETLRLHPPTVFLLPRKAEANVQLYGYLIPKNAQLLVNLWAIGRDPNVWNDPLVFSPERFLASSSCEIDVKGRHFELLPFGAGRRICPGLTLAYRMLNLMLVTLVNSFDWKLEDVTSAKDLDMDEKFGITLQKVKPLQLIPIPK

SEQ ID NO: 41 - DNA, 1485 bp, Abronia nealleyi
  source 1..1485: mol_type = other DNA
atggaccaaactaccttggccatgttgttgtctgcattatacctgctgtacaacctgtacaaggttatcttcactcagtccaattctaaattgccaccaggtccaaaaccattgccaatttttggtaacatctctgaattgggtgctaagccacatagatcttttgctaacttggctaagattcacggtccattgattactttgaagttgggttctgttacgaccatcgttgtttcttcagctaaagttgccgaagagatgttcttgaagaatgatttgccattggccaacagaaacgttccaaattctgttactgctggtgatcatcataagttgactatgtcttggttgccagtttctccaaagtggaaaaccttcagaaagattaccgctgttcatttgttgtccccacaaagattggatgcttgtcaagctttgagacatgctaaagttaagcagttgtacgaatacgtttacgattgtgctaaaaagggtgaagccgttgatattggtaaagctgcttttactacctccttgaacttgctgtctaacttgttcttctctgttgaattggctcaacatacctctacctcttcacaacatttcaagcaattgatctgggacatcatggaagatatcggtaagccaaattacgctgattatttcccagctttgaagtgtgttgatccatggggtattagaagaagattggctgctaatttcgaaaggttgatcgatgttttccaggacttcattagaccaaggttgtctatgaatccatcctctgttacttcagcttctgatgttttggatgtcctgctgaacttgtacaaagaaaaagaattgaacatgggcgaagtcaaccacttgttggttgatatttttgatgctggtactgacaccacttcttctacatttgaatgggctatggctgaattggttagacatccagaaactatgaagaaggcccaagacgaaattgaacaagttttgggtaaagatgccaccattcaagaagctgatattccaaaaatgccatacttgcaggccatcatcaaagaaactttgagattgcatccaccaaccgtttttttgttgccaagaaaagctgctaccaacgtcgaattatatggttacgttgttccaaaggatgcccaaatcttggttaatttgtgggctattggtagagatccattggtttgggatcaacctaatgttttctcaccagaaaggttcttgaactccgatgttgatgttaagggtagagattttggtttgttgccttttggtgcaggtagaagaatttgtccaggtatgaatttggcctacagaatgttgactttgatgttggctactctgttgcaatctttcgaatggaaggttgaaaatggtgaaaaggccgaagatttggatatggacgaaaaatttggtatcgccttgcaaaagactaagccattgcaaattattcccgtcctgaagtactga

SEQ ID NO: 42 - protein, 494 aa, Abronia nealleyi
MDQTTLAMLLSALYLLYNLYKVIFTQSNSKLPPGPKPLPIFGNISELGAKPHRSFANLAKIHGPLITLKLGSVTTIVVSSAKVAEEMFLKNDLPLANRNVPNSVTAGDHHKLTMSWLPVSPKWKTFRKITAVHLLSPQRLDACQALRHAKVKQLYEYVYDCAKKGEAVDIGKAAFTTSLNLLSNLFFSVELAQHTSTSSQHFKQLIWDIMEDIGKPNYADYFPALKCVDPWGIRRRLAANFERLIDVFQDFIRPRLSMNPSSVTSASDVLDVLLNLYKEKELNMGEVNHLLVDIFDAGTDTTSSTFEWAMAELVRHPETMKKAQDEIEQVLGKDATIQEADIPKMPYLQAIIKETLRLHPPTVFLLPRKAATNVELYGYVVPKDAQILVNLWAIGRDPLVWDQPNVFSPERFLNSDVDVKGRDFGLLPFGAGRRICPGMNLAYRMLTLMLATLLQSFEWKVENGEKAEDLDMDEKFGIALQKTKPLQIIPVLKY

SEQ ID NO: 43 - DNA, 1494 bp, Spinacia oleracea
  source 1..1494: mol_type = other DNA
atggaagataccaccttggccatcttgttgtctatttctttcgtttgcttccacgtcatcaggtcctttgtttctaagaccagatccaagtcatctgaattgccaccaggtccaaaaagaatgccaattttcggtaacatcttcgacttgggtgataagccacatagatcttttgctaacttgtccaagattcacggtccattggtttctttgaagttgggttctatcactaccatcgttgtttcttcagctgatgttgctagagaaatgttcttgaagaatgatttggctttggccaacagaaccattccagattctgttagagctggtgatcatgacaaattgtctatgtcttggttgccagtttctgctaaatggcgtaacttgagaaagatttctgctgtccagctgttgtccaatcaaagattggatgcttctcaagctcatagacaagctaaagttgaacagttgttgacctacgttaaggactgttctaaaaacggtttgccagttgatattggtagagctgcttttactacctccttgaacttgttgtcaaacaccttcttctctgttgaattggcctctcatgaatcttcagcttcccaagaattcaagcaattgatgtggaacatcatggaagaaatcggtaagccaaattacgctgattacttcccaattttgggttacgttgatccattcggtatcagaagaagattggctgcatacttcgaccaattgattgctgttttccagaacatcatccaagagagacaaaagattagatccaccaacgctaagcaaactaacgatattttggacaccttgttgaacttgcacgacgaaaatgaattatccatgggtgaaatcaaccacttgttggttgatattttcgatgctggtactgataccactgcttctactttggaatgggctatggctgaattggttaagaatccagaaatgatgaccaaggtccagaacgaaattgaacaagctattggtaaggattgcgccaccattcaagaatctgatattgctaaattgccatacttgcaggccattatcaaagagactttgagattgcatccacctaccgtttttttgttgccaagaaaagcagatgttgacgttgagttgtacggttatgttgttccaaagaacgctcaagttttggttaacttgtgggctataggtagagatccaaaggtttggaaaaacccagaagttttctcaccagaaaggttcttggaatgcgatattgattacaagggtagagacttcgagttgttgccatttggtgctggtagaagaatttgtccaggtttgactttggcatacaggatgttgaatttgatgatgggtaacttcttgcactccttcgattggaaattggaagatggtatgtctccaaaggacttggatatggacgaaaagtttggtatcaccttgcaaaaggttaagccattgcaagttattccagtgccaagatga

SEQ ID NO: 44 - protein, 497 aa, Spinacia oleracea
MEDTTLAILLSISFVCFHVIRSFVSKTRSKSSELPPGPKRMPIFGNIFDLGDKPHRSFANLSKIHGPLVSLKLGSITTIVVSSADVAREMFLKNDLALANRTIPDSVRAGDHDKLSMSWLPVSAKWRNLRKISAVQLLSNQRLDASQAHRQAKVEQLLTYVKDCSKNGLPVDIGRAAFTTSLNLLSNTFFSVELASHESSASQEFKQLMWNIMEEIGKPNYADYFPILGYVDPFGIRRRLAAYFDQLIAVFQNIIQERQKIRSTNAKQTNDILDTLLNLHDENELSMGEINHLLVDIFDAGTDTTASTLEWAMAELVKNPEMMTKVQNEIEQAIGKDCATIQESDIAKLPYLQAIIKETLRLHPPTVFLLPRKADVDVELYGYVVPKNAQVLVNLWAIGRDPKVWKNPEVFSPERFLECDIDYKGRDFELLPFGAGRRICPGLTLAYRMLNLMMGNFLHSFDWKLEDGMSPKDLDMDEKFGITLQKVKPLQVIPVPR

SEQ ID NO: 45 - DNA, 1503 bp, Spinacia oleracea
  source 1..1503: mol_type = other DNA
atggaaaacaccaccttggccttgatcttgtctatttcttttgtttgcttgcacgtcatcaggtcttttttgggtaaagcttcctccaagtcatctaaattgccaccaggtccaaaaagaatgccaattttcggtaacatcttcgacttgggtgaaaagccacatagatcttttgctaacttggctaagattcacggtccattggtttctttgaagttgggttctatcactaccatcgttgtttcatctgctgaagttgccaaagaaatgttcttgaagaacgatcaagttttggccaacagaaccattccagattctgttagagctggtgatcatgacaaattgtctatgtcttggttgccagtttctgctaaatggcgtaacttgagaaagatttctgctgtccagttgttgtctaaccagagattggatgcttctcaagctcatagacaagctaaagttgaacagttgttgacctacgttcaagactgttctaaaaagggtttgccagttgatattggtagagctgcttttactacctccttgaacttgttgtccaacacctttttctctgttgaattggcctctcatgaatcctctgcttcacaagaattcaagcaattgatgtggaacatcatggaagaaatcggtaagccaaattacgctgattacttcccaattttgggttacgttgatccattcggtatcagaagaagattggctgcttacttcgaccaattgattgctgttttccagaacatcatccaagagagacaaaagattaggtctaccaatggttctaacgccaagcaaactaacgatatcttggataccttgttgaacttgcacgacgaaaacgaattatccatgggtgaaatcaaccacttgttggttgatattttcgatgctggtactgataccactgcttctactttggaatgggctatggctgaattgattaagaacccagaaatgatgaccaaggtccagaacgaaattgaacaagctgttggtaaagattgctccgccattcaagaatctgatattgcaaaattgccctacctgcagtccattatcaaagagactttgagattgcatccaccaaccgtttttttgttgccaagaaaagctgatgttgacgttgagttgtacggttatgttgttccaaagaatgctcaagtcttggttaacttgtgggctataggtagagatccaaaggtttggaaaaacccagagattttctcaccagaaaggttcttggaatgcgatattgattacaagggtagagacttcgagttgttgccatttggtgctggtagaagaatttgtccaggtttgactttggcatacaggatgttgaatttgatgatgggtaacttcttgcactccttcgattggaaattggaagatggtatgaacccaaaggacttggatatggacgaaaagtttggtatcaccttgcaaaaggttaagccattgcaagttatcccagtctctagatga

SEQ ID NO: 46 - protein, 500 aa, Spinacia oleracea
MENTTLALILSISFVCLHVIRSFLGKASSKSSKLPPGPKRMPIFGNIFDLGEKPHRSFANLAKIHGPLVSLKLGSITTIVVSSAEVAKEMFLKNDQVLANRTIPDSVRAGDHDKLSMSWLPVSAKWRNLRKISAVQLLSNQRLDASQAHRQAKVEQLLTYVQDCSKKGLPVDIGRAAFTTSLNLLSNTFFSVELASHESSASQEFKQLMWNIMEEIGKPNYADYFPILGYVDPFGIRRRLAAYFDQLIAVFQNIIQERQKIRSTNGSNAKQTNDILDTLLNLHDENELSMGEINHLLVDIFDAGTDTTASTLEWAMAELIKNPEMMTKVQNEIEQAVGKDCSAIQESDIAKLPYLQSIIKETLRLHPPTVFLLPRKADVDVELYGYVVPKNAQVLVNLWAIGRDPKVWKNPEIFSPERFLECDIDYKGRDFELLPFGAGRRICPGLTLAYRMLNLMMGNFLHSFDWKLEDGMNPKDLDMDEKFGITLQKVKPLQVIPVSR

SEQ ID NO: 47 - DNA, 1497 bp, Spinacia oleracea
  source 1..1497: mol_type = other DNA
atggaaaacaccaccttggccttcatcttgtctatttctttcgtttgcttccacgtcatcaggtcctttatttctaaggccagatccaagtcatctaaattgccaccaggtccaaaaagaatgccaattttcggtaacatcttcgacttgggtgaaaagccacatagatcttttgctaacttgtccaagattcacggtccattgatttctttgaagttgggttctatcacgaccatcgttgtttcttcagctgatgttgctaaagaaatgttcttgaagaacgatcaagctttggccaacagaactattccagattctgttagagctggtgatcacgataagttgtcaatttcttggttgccagtttctgctaagtggcgtaatttgagaaagatttctgctgtccagttgttgtctaaccagagattggatgcttctcaagctcatagacaagctaaagttgaacagttgttgacctacgttaaggactgttctaaaaagggtttgccagttgatattggtagagctgcttttactacctccttgaacttgttgtccaacacctttttctctgttgaattggcctctcatgaatcctctgcttcacaagaattcaagcaattgatgtggaacatcatggaagaaatcggtaagccaaactacgctgattactttccaattttgggttacgttgatccattcggtatcagaagaagattggctgcttacttcgaccaattgattgctgttttccaagacatcatccagaagagacaaaagattaggtctaccaatggtgccaagcaaactaacgatattttggacactttgctgaacttgcacgaagaaaacgaattgtctatgggtgaaatcaaccacttgttggttgatattttcgatgctggtactgataccactgcttctactttggaatgggctatggctgaattggttagaaatccagaaatgatgaccaaggtccagaacgaaattgaacaagctattggtaagaactgcgccaccattcaagaatctgatattccaaaattgccatacttgcaggccatcatcaaagaaactttgagattgcatccaccaaccgtctttttgttgccaagaaaagcagatgttgatgtcgagttgtacggttatgttgttccaaagaacgcacaagttttggttaacttgtgggctataggtagagatccaaaggtttggaagaactccgaagttttttcaccagaaagattcttggattgcgacatcgattataagggtagagattttgagttgttgccatttggtgctggtagaagaatttgtccaggtttgactttggcatacaggatgttgaacttaatgatgggtaacttcttgcactccttcgattggaaattggaagatggtatgtctccaaaggacttggatatggacgaaaagtttggtatcaccttgcaaaaggttaagccattgcaagttattcccgttccaaagtga

SEQ ID NO: 48 - protein, 498 aa, Spinacia oleracea
MENTTLAFILSISFVCFHVIRSFISKARSKSSKLPPGPKRMPIFGNIFDLGEKPHRSFANLSKIHGPLISLKLGSITTIVVSSADVAKEMFLKNDQALANRTIPDSVRAGDHDKLSISWLPVSAKWRNLRKISAVQLLSNQRLDASQAHRQAKVEQLLTYVKDCSKKGLPVDIGRAAFTTSLNLLSNTFFSVELASHESSASQEFKQLMWNIMEEIGKPNYADYFPILGYVDPFGIRRRLAAYFDQLIAVFQDIIQKRQKIRSTNGAKQTNDILDTLLNLHEENELSMGEINHLLVDIFDAGTDTTASTLEWAMAELVRNPEMMTKVQNEIEQAIGKNCATIQESDIPKLPYLQAIIKETLRLHPPTVFLLPRKADVDVELYGYVVPKNAQVLVNLWAIGRDPKVWKNSEVFSPERFLDCDIDYKGRDFELLPFGAGRRICPGLTLAYRMLNLMMGNFLHSFDWKLEDGMSPKDLDMDEKFGITLQKVKPLQVIPVPK

SEQ ID NO: 49 - DNA, 1503 bp, Chenopodium quinoa
  source 1..1503: mol_type = other DNA
atggactctaccaccttggttatggttgtcatttccatcttgttcgtgttcttgtaccacgtcaagtcattcttcatcaggtacttctctaatagattgccaccaggtccaaaacctaagccaatttttggtaacatcttcgacttgggtgaaaagccacatagatcttttgctaacttggctaagattttcggtcccttgatttctttgaagttgggtaatgttactaccgtcgttgtctcttcttcatacgttgctgaagaaatgttcttgaagaacgatcaatccttcgccaacagaactattccagattctgttagagctggtaaccacgataagttgtctatgtcttggttgccaatttctccacaatggcgtaacttgagaaagatttccgctgtccaattattgtctacccaaagattggatgcttcccaagctttgagacaagctaaagtttctcaattgcatgcctacgttcaagactgttctaaaaagttgcaaccagtcaacattggtagagctgcttttactacttccctgaacttgttgtctaacaccttcttctctattgaattggcctctcatgaatcctctacctctcaagaattcaagcaattgatgtggaacatcatggaagaaatcggtagaccaaattacgctgattacttcccaatcttgggttacattgatccattcggtatcagaagaagattggcttcttacttcgatgaactgatcgtcgttttccaaaacatcatttgcgaaaggcagaacatcagatcctctgatgattcttctgctaagcacactaacgatgtcttggatactttgttgaacctgtacgacaagaacgaattgtctatggacgaaatcaaccacttgttggttgatattttcaacgctggtactgataccactgcttctactttggaatggactatgaccgagttgattaagaacccaaagtccatgattatctgccagaacgaaattgaacaagccttaggtaaaggctccttgtccattcaagaatccgatatttctaagctgccatacttgcaagccattatgaaggaaactttgagattgcatccaccaaccgtttttttgttgccaagaaaagctgattccgatgttgaattgtgtggttacgttgttcctaaacatgcccaagttttggttaacttgtgggctataggtagagatccaaaggtttgggaaaatccagaagttttctcaccagaaagattcttggattgcgaaattgacttgaagggtcaagattttgagttgttgccatttggtgctggtagaagaatttgtccaggtttgaatttggcctacaggatgttgaacttgatgttggctactctgttgcataactacaactggaaattggaagatggcatgaccttgaacgatttggatatggatgagaagttcggtattaccttgcaaaaggttaagccattgcaagttgttccaattccaaggtactga

SEQ ID NO: 50 - protein, 500 aa, Chenopodium quinoa
MDSTTLVMVVISILFVFLYHVKSFFIRYFSNRLPPGPKPKPIFGNIFDLGEKPHRSFANLAKIFGPLISLKLGNVTTVVVSSSYVAEEMFLKNDQSFANRTIPDSVRAGNHDKLSMSWLPISPQWRNLRKISAVQLLSTQRLDASQALRQAKVSQLHAYVQDCSKKLQPVNIGRAAFTTSLNLLSNTFFSIELASHESSTSQEFKQLMWNIMEEIGRPNYADYFPILGYIDPFGIRRRLASYFDELIVVFQNIICERQNIRSSDDSSAKHTNDVLDTLLNLYDKNELSMDEINHLLVDIFNAGTDTTASTLEWTMTELIKNPKSMIICQNEIEQALGKGSLSIQESDISKLPYLQAIMKETLRLHPPTVFLLPRKADSDVELCGYVVPKHAQVLVNLWAIGRDPKVWENPEVFSPERFLDCEIDLKGQDFELLPFGAGRRICPGLNLAYRMLNLMLATLLHNYNWKLEDGMTLNDLDMDEKFGITLQKVKPLQVVPIPRY

SEQ ID NO: 51 - DNA, 1497 bp, Spinacia oleracea
  source 1..1497: mol_type = other DNA
atggactacaccaccttggtcatcatcttgtctattatgttcgtttgcttccacctgttgaagtcttttttcactactccatcctctcataagttgccaccaggtccaaaacctattccaattttcggtaacatcttctacttgggtgaaaagccacatagatcttttgctaacttggctaagattcacggtccattgatttctttgaagttgggttctgttacgacgatcgttgtttcttcttcatacgttgctgaagagatgttcttgaaacatgatagagctttcgccaacagaaccattccagattctattagagcttgcgatcacgacaaattgtctatgtcttggttgcccatttccttgaattggagaaacttgaggaagatctcttccgttcagttgttgtccaatcaaagattggatgcttctcaagctcatagacaagctcaagtcgaacaattattggcttacgttcaagactgctccattaagggtattccagttgatattggtagagctgcttttaccacttccttgaacttgttgtctaacaccttcttctctgttgaattggcctctcatgaatcttcagcttcccaagaattcaagcaattgatgtggaacatcatggaagaaatcggtaagccaaattacgctgattacttcccaattttggcctacgtagatccatttggtgttagaagaagattggctgcctacttcgaccaattgattgatgttttccaggacatcatcaaagagcgtcaaaagatcagattgaagaacggttcatctgccaagcaaactaacgatattttggacaccttgttgaacttgcacgacgaaaatgaattatccatgggtgaaatcaaccacttgttggttgatattttcaacgctggtactgataccactgcttctactttggaatgggctatgactgaattggttagaaaccctagaatcatgggtagagttcaacacgaaattgaacaagctttggaaaaggactgctcctctattcaagaagcctacattttgaaactgccatacttgcaggccattatcaaagaaactttgagattgcatccaccaaccgtttttttgttgccaagaaaggctaacatcgaagtcgaattgcatggttttactgttccaaagaacgcccaaatcttggttaatttgtgggctgttggtagagatccaaaggtttgggaaaatcctaaggttttcatcccagacagattcttggaatgcgatattgatgttaagggtcagaactttgagttgttgccttttggtgctggtagaagaatttgtccaggtttgggtttagcttacaggatgttgaatttgatcttggccaccttgatccataactacgattggaaattggaggatggtatgaacgttaacgatttggatatggacgaaaagttcggtatcaccttgcaaaaagttgttccattgaaggttatccccgttccaagataa

SEQ ID NO: 52 - protein, 498 aa, Spinacia oleracea
MDYTTLVIILSIMFVCFHLLKSFFTTPSSHKLPPGPKPIPIFGNIFYLGEKPHRSFANLAKIHGPLISLKLGSVTTIVVSSSYVAEEMFLKHDRAFANRTIPDSIRACDHDKLSMSWLPISLNWRNLRKISSVQLLSNQRLDASQAHRQAQVEQLLAYVQDCSIKGIPVDIGRAAFTTSLNLLSNTFFSVELASHESSASQEFKQLMWNIMEEIGKPNYADYFPILAYVDPFGVRRRLAAYFDQLIDVFQDIIKERQKIRLKNGSSAKQTNDILDTLLNLHDENELSMGEINHLLVDIFNAGTDTTASTLEWAMTELVRNPRIMGRVQHEIEQALEKDCSSIQEAYILKLPYLQAIIKETLRLHPPTVFLLPRKANIEVELHGFTVPKNAQILVNLWAVGRDPKVWENPKVFIPDRFLECDIDVKGQNFELLPFGAGRRICPGLGLAYRMLNLILATLIHNYDWKLEDGMNVNDLDMDEKFGITLQKVVPLKVIPVPR

SEQ ID NO: 53 - DNA, 1491 bp, Mirabilis jalapa
  source 1..1491: mol_type = other DNA
atggacttcttgaccctggtcatgatcttgtctattatcttcttcttctacaacctgctgaagatgaagttcactactcattctgatgctcaattgccaccaggtccaaaacctatgccaatttttggtaacatcttcgagttgggtgaaaagccacatagatcttttgctaacttggctaaaactcacggtccattgatgtctttgagattgggttctgttactaccatcgttgtttcatctgctgaagttgccaaagaaatgttcttgaagaacgatcaatccttggccgatagatctgttccaaattctgttacagctggtgatcatcataagttgactatgtcttggttgccagtttctccaaagtggaagaacttcagaaagattaccgctgttcatttgttgtccccacaaagattggatgcttgtcatgctttgagacatgctaaagttaagcagttgtacgaatacgttcaagaatgtgctttgaagggtgaagctgttgatattggtaaagctgctttcaccacttccttgaacttgttgtctaacttgttcttctccgttgaattggctaaccatacttctaacacctctcaagaattcaagcaattgatctgggacatcatggaagatatcggtaagccaaattacgctgattacttccccttgttgaaatacgttgatccatccggtattagaagaaggttggctgctaatttcgataagttgatcgacgttttccagtccttcatctctaagagattatcctctgcttactcttctgctacctctttggatgatgttttggatgtcttgttgaagttgttgaaagaaaaagaactgaacatgggcgagatcaaccatttgttggttgatatttttgatgccggtactgataccacctctaatacttttgaatgggctatggctgaattgatgagaaacccaattatgatgaagagagcccaaaacgaaattgccttggttttgggtaaagataacgccaccattcaagaatccgatattgctaatatgccatacttgcaggccatcatcaaagaaacattgagattgcatccaccaaccgtttttttgttgccaagaaaggctattaccaacgtcaagttgtatggttacatcgttccaaagaacgcccaaatcttggttaatttgtgggctattggtagagatccaaaggtttggaagaatccaaacgaattcttgccagataggttcttgaactctgatatcgatgttaagggtagagactttggtttgttaccatttggtgctggtagaagaatttgtccaggtatgaatttggcctacagaatgttgactttgatgttggctactctgttgcaatctttcgattggaagttgcctcatagaaactctccattggatttggatatggacgaaaagtttggtatcgccttgcaaaaaactaagccattggaaatcatcccactgatcaagtactga

SEQ ID NO: 54 - protein, 496 aa, Mirabilis jalapa
MDFLTLVMILSIIFFFYNLLKMKFTTHSDAQLPPGPKPMPIFGNIFELGEKPHRSFANLAKTHGPLMSLRLGSVTTIVVSSAEVAKEMFLKNDQSLADRSVPNSVTAGDHHKLTMSWLPVSPKWKNFRKITAVHLLSPQRLDACHALRHAKVKQLYEYVQECALKGEAVDIGKAAFTTSLNLLSNLFFSVELANHTSNTSQEFKQLIWDIMEDIGKPNYADYFPLLKYVDPSGIRRRLAANFDKLIDVFQSFISKRLSSAYSSATSLDDVLDVLLKLLKEKELNMGEINHLLVDIFDAGTDTTSNTFEWAMAELMRNPIMMKRAQNEIALVLGKDNATIQESDIANMPYLQAIIKETLRLHPPTVFLLPRKAITNVKLYGYIVPKNAQILVNLWAIGRDPKVWKNPNEFLPDRFLNSDIDVKGRDFGLLPFGAGRRICPGMNLAYRMLTLMLATLLQSFDWKLPHRNSPLDLDMDEKFGIALQKTKPLEIIPLIKY

SEQ ID NO: 55 - DNA, 1491 bp, Mirabilis multiflora
  source 1..1491: mol_type = other DNA
atggacttcttgaccctggtcatgatcttgtctatgatcttcttcttctacaacctgctgaagatgattttcactacccattctgatgctcaattgccaccaggtccaaaacctatgccaatttttggtaacatcttcgagttgggtgaaaagccacatagatcttttgctaacttggctaaaactcacggtccattgatgtctttgagattgggtagagttactaccatcgttgtttcatctgctgaagttgccaaagaaatgttcttgaagaacgatcaatccttggccgatagatctgttccaaattctgttactgctggtgatcatcataagttgactatgtcttggttgccagtttctccaaagtggaagaacttcagaaagattaccgctgttcatttgttgtccccacaaagattggatgcttgtaatgctttgagacatgctaaggttaagcagttgtacgaatacgttcaagaatgtgctttgaagggtgaagctgttgatattggtaaagctgctttcaccacttccttgaacttgttgtctaacttgttcttctccgttgaattggctaaccatacttctaacacctctcaagaattcaagcaattgatctgggacatcatggaagatatcggtaagccaaattacgctgattacttccccttgttgaaatacgttgatccatccggtattagaagaaggttggctgctaatttcgataagttgatcgacgttttccagtccttcatctgtaagagattatcctctgcttactcttctgctacctctttggatgatgttttggatgtcttgttgaagctgtacaaagaaaaagaattgaacatgggcgagatcaaccacttgttggttgatatttttgatgccggtactgataccacctctaatacttttgaatgggccatgtccgaattgattagaaacccaactatgatgaagagagcccaaaacgaaattgctttggttttgggtaaagataacggcaccattcaagaatccgatattgctaatatgccatacttgcaggccatcatcaaagaaacattgagattgcatccaccaaccgtttttttgttgccaagaaaggctattacctacgtcaagttgtatggttacatcgttccaaagaacgcccaaatcttggttaatttgtgggctattggtagagatccaaaggtttggaagaatccaaacgaattcttgccagataggttcttgaactctgatatcgatgttaagggtagagactttggtttgttaccatttggtgctggtagaagaatttgtccaggtatgaatttggcctacagaatgttgactttgatgttggctactctgttgcaatctttcgattggaaattgccacatggtaactctccaatggatttggatatggacgaaaagtttggtatcgccttgcaaaaaactaagcccttggaaattatccccgtcatcaagtactaa

SEQ ID NO: 56 - protein, 496 aa, Mirabilis multiflora
MDFLTLVMILSMIFFFYNLLKMIFTTHSDAQLPPGPKPMPIFGNIFELGEKPHRSFANLAKTHGPLMSLRLGRVTTIVVSSAEVAKEMFLKNDQSLADRSVPNSVTAGDHHKLTMSWLPVSPKWKNFRKITAVHLLSPQRLDACNALRHAKVKQLYEYVQECALKGEAVDIGKAAFTTSLNLLSNLFFSVELANHTSNTSQEFKQLIWDIMEDIGKPNYADYFPLLKYVDPSGIRRRLAANFDKLIDVFQSFICKRLSSAYSSATSLDDVLDVLLKLYKEKELNMGEINHLLVDIFDAGTDTTSNTFEWAMSELIRNPTMMKRAQNEIALVLGKDNGTIQESDIANMPYLQAIIKETLRLHPPTVFLLPRKAITYVKLYGYIVPKNAQILVNLWAIGRDPKVWKNPNEFLPDRFLNSDIDVKGRDFGLLPFGAGRRICPGMNLAYRMLTLMLATLLQSFDWKLPHGNSPMDLDMDEKFGIALQKTKPLEIIPVIKY

SEQ ID NO: 57 - DNA, 1500 bp, Beta vulgaris
  source 1..1500: mol_type = other DNA
atggatcatgctaccttggctatgattttggccatcttgttcattagcttccacttcatcaagctgttgttctctcaacaaactaccaagttgttgccaccaggtccaaaaccattgccaattattggtaacatcttggaggttggtaagaagccacatagatcttttgctaatttggccaagattcacggtccattgatttctttgagattgggttctgttacgaccatcgttgtttcttcagctgatgttgctaaagagatgttcttgaagaaggatcacccattgtccaacagaactattccaaattctgttaccgctggtgatcatcataagttgactatgtcttggttgccagtttctccaaagtggcgtaatttcagaaagattaccgctgttcatttgttgtccccacaaagattggatgcttgtcaaacttttagacacgctaaggttcaacagttgtacgaatacgttcaagaatgtgctcaaaaaggtcaagccgttgatattggtaaagctgcttttactacctccttgaacttgctgtctaagttgttcttctctgttgaattggctcatcacaagtctcatacctctcaagaattcaaagagctgatctggaacatcatggaagatatcggtaagccaaattacgctgattacttcccaattttgggttgcgttgatccatctggtattagaagaagattggcttgctctttcgataagttgattgctgttttccaaggtatcatctgtgaaagattagccccagattcttctactactacaactactaccactgatgatgttttggatgtgttgttgcagttgttcaagcaaaacgaattgaccatgggtgaaatcaaccacttgttggttgatattttcgatgctggtactgataccacttcctctactttggaatgggttatgaccgaattgatcagaaacccagaaatgatggaaaaggcccaagaagagattaagcaagttttgggtaaagacaagcagatccaagaatccgatattatcaacttgccatacttgcaggccatcatcaaagaaacattgagattgcatccaccaaccgtttttttgttgccaagaaaagctgataccgatgttgagttgtatggttacatcgttccaaaggatgcccaaatcttggttaatttgtgggctattggtagagatccaaatgcttggcaaaacgccgatattttctcaccagaaaggttcattggttgcgaaattgatgttaagggtagagactttggtttgttgccttttggtgctggtagaaggatttgtccaggtatgaatttggctatcagaatgttgactttgatgctggctactctgttgcaatttttcaactggaaattggagggtgacatctcaccaaaagatttggatatggacgaaaagttcggtatcgccttgcaaaaaactaagccattgaagttgatccccattcctagatacggttcttga

SEQ ID NO: 58 - protein, 499 aa, Beta vulgaris
MDHATLAMILAILFISFHFIKLLFSQQTTKLLPPGPKPLPIIGNILEVGKKPHRSFANLAKIHGPLISLRLGSVTTIVVSSADVAKEMFLKKDHPLSNRTIPNSVTAGDHHKLTMSWLPVSPKWRNFRKITAVHLLSPQRLDACQTFRHAKVQQLYEYVQECAQKGQAVDIGKAAFTTSLNLLSKLFFSVELAHHKSHTSQEFKELIWNIMEDIGKPNYADYFPILGCVDPSGIRRRLACSFDKLIAVFQGIICERLAPDSSTTTTTTTDDVLDVLLQLFKQNELTMGEINHLLVDIFDAGTDTTSSTLEWVMTELIRNPEMMEKAQEEIKQVLGKDKQIQESDIINLPYLQAIIKETLRLHPPTVFLLPRKADTDVELYGYIVPKDAQILVNLWAIGRDPNAWQNADIFSPERFIGCEIDVKGRDFGLLPFGAGRRICPGMNLAIRMLTLMLATLLQFFNWKLEGDISPKDLDMDEKFGIALQKTKPLKLIPIPRYGS

SEQ ID NO: 59 - DNA, 1419 bp, unidentified
  source 1..1419: mol_type = other DNA
atgaccccagaacaattcagacaatacggtcaccaattgattgatttgatcgccgattacagacaaaccgttggtgaaagaccagttatggctcaagttgaaccaggttatttgaaagctgctttgccagctactgctccacaacaaggtgaaccatttgctgctattttggatgatgttaacaacttggttatgccaggtttgtctcattggcaacatccagatttttacggttactttccatccaacggtactttgtcatctgttttgggtgatttcttgtctactggtttgggtgttttaggtttgtcatggcaatcttctccagctttgtcagaattggaagaaactactttggattggttgagacagttgttgggtttatctggtcaatggtctggtgttattcaagatactgcttctacttctaccttggttgctttgatttctgctagagaaagagctactgattacgctttggttagaggtggtttacaagctgaacctaaaccattgatcgtttacgtttctgctcatgcccattcttcagttgataaggctgctttgttggctggttttggtagagataacattagattgattccaaccgacgaaagatacgccttaagaccagaagctttacaagcagctattgaacaagatttggctgctggtaatcaaccatgtgctgttgttgctactactggtactactactacaactgctttggatccattaagacctgtaggtgaaattgctcaagctaatggtttgtggttgcatgttgattcagctatggctggttctgctatgattttgccagaatgtagatggatgtgggatggtattgaattggctgattctgttgttgttaacgcccataagtggttgggtgttgcttttgattgctctatctactacgttagagatccacaacacttgatcagagtcatgtctactaatccatcctacttgcaatctgctgttgatggtgaagttaagaacttgagagattggggtattccattgggtagaagattcagagctttgaagttgtggtttatgttgagatccgaaggtgttgatgcattgcaagctagattgagaagagatttggataatgctcaatggttggctggacaagttgaagctgctgctgaatgggaagttttggctccagttcaattgcaaaccttgtgcattagacatagaccagcaggtttggaaggtgaagctttggatgctcatacaaaaggttgggctgaaagattgaatgcttctggtgctgcttatgttactccagctactttagatggaagatggatggttagagtttccattggtgctttaccaactgaaagaggtgacgttcaaagattgtgggctagattacaagatgtcatcaaaggtggttcctaa

SEQ ID NO: 60 - protein, 472 aa, unidentified
MTPEQFRQYGHQLIDLIADYRQTVGERPVMAQVEPGYLKAALPATAPQQGEPFAAILDDVNNLVMPGLSHWQHPDFYGYFPSNGTLSSVLGDFLSTGLGVLGLSWQSSPALSELEETTLDWLRQLLGLSGQWSGVIQDTASTSTLVALISARERATDYALVRGGLQAEPKPLIVYVSAHAHSSVDKAALLAGFGRDNIRLIPTDERYALRPEALQAAIEQDLAAGNQPCAVVATTGTTTTTALDPLRPVGEIAQANGLWLHVDSAMAGSAMILPECRWMWDGIELADSVVVNAHKWLGVAFDCSIYYVRDPQHLIRVMSTNPSYLQSAVDGEVKNLRDWGIPLGRRFRALKLWFMLRSEGVDALQARLRRDLDNAQWLAGQVEAAAEWEVLAPVQLQTLCIRHRPAGLEGEALDAHTKGWAERLNASGAAYVTPATLDGRWMVRVSIGALPTERGDVQRLWARLQDVIKGGS

SEQ ID NO: 61 - protein, 196 aa, Camellia japonica
MRMEVVLVVFLMFIGTINCERLIFNGRPLLHRVTKEETVMLYHELEVAASADEVWSVEGSPELGLHLPDLLPAGIFAKFEITGDGGEGSILDMTFPPGQFPHHYREKFVFFDHKNRYKLVEQIDGDFFDLGVTYYMDTIRVVATGPDSCVIKSTTEYHVKPEFAKIVKPLIDTVPLAIMSEAIAKVVLENKHKSSE

SEQ ID NO: 62 - protein, 346 aa, Papaver somniferum
METVSKIDQQNQAKIWKQIYGFAESLVLKCAVQLEIAETLHNNVKPMSLSELASKLPVAQPVNEDRLFRIMRYLVHMELFKIDATTQKYSLAPPAKYLLRGWEKSMVDSILCINDKDFLAPWHHLGDGLTGNCDAFEKALGKSIWVYMSVNPEKNQLFNAAMACDTRLVTSALANECKSIFSDGISTLVDVGGGTGTAVKAISKAFPDIKCTIYDLPHVIADSPEIPNITKISGDMFKSIPSADAIFMKCILHDWNDDECIQILKRCKEALPKGGKVIIVDVVIDMDSTHPYAKIRLTLDLDMMLNTGGKERTKEEWKTLFDAAGFASHKVTQISAVQSVIEAYPY

SEQ ID NO: 63 - protein, 350 aa, Papaver somniferum
MQLKAKEELLRNMELGLIPDQEIRQLIRVELEKRLQWGYKETHEEQLSQLLDLVHSLKGMKMATEMENLDLKLYEAPMEFLKIQHGSNMKQSAGYYTDESTTLDEAEIAMLDLYMERAQIKDGQSVLDLGCGLGAVALFGANKFKKCQFTGVTSSVEQKDYIEGKCKELKLTNVKVLLADITTYETEERFDRIFAVELIEHMKNYQLLLKKISEWMKDDGLLFVEHVCHKTLAYHYEPVDAEDWYTNYIFPAGTLTLSSASMLLYFQDDVSVVNQWTLSGKHYSRSHEEWLKNMDKNIVEFKEIMRSITKTEKEAIKLLNFWRIFCMCGAELFGYKNGEEWMLTHLLFKK

SEQ ID NO: 64 - DNA, 1613 bp, Eschscholzia californica
  misc_difference 612: note = n is a, c, g, or t
  source 1..1613: mol_type = other DNA
aagtccgagtgagaagcagagttagagaaaaaaaaaatggaggttgtcacagtagcacttattgcagtaataatttcttcaatactctacctcctctttggtagtagtggtcacaaaaatctcccaccaggaccaaaaccatggccaatagtaggaaatcttctccaacttggtgagaaaccacacgctcaattcgccgaactagctcaaacctatggtgacattttcactcttaaaatgggtactgaaactgtagttgttgcatcaacatcttcggcagcttccgaaatactaaaaacccatgatcgaattctatccgctcgttacgtttttcaaagttttcgagtaaaagggcatgtagaaaattcaatagtttggtcagattgtactgaaacttggaagaatttaagaaaagtttgtaggacggaacttttcacacagaagatgatagaaagtcaagctcatgttagagagaaaaaatgtgaagaaatggttgaatacttgatgaaaaaacaaggggaagaagtgaaaattgtggaagtaatatttggaacattagtgaatatattcggaaatttgatattttcacagaatatatttgaattgggtganccaaatagtggaagttcagagttcaaggaatatctatggaggatgttggaattagggaattcaacaaatccagctgattattttccaatgttgggtaaatttgatttgtttggacagaggaaagaagttgcagagtgtttaaaagggatttatgctatttggggagctatgcttcaagaaaggaaattagctaaaaaagttgatggataccaaagcaagaatgattttgttgatgtttgtcttgattctggacttaatgattatcagatcaatgccttgcttatggaattatttggggcaggcacagaaacaagcgcatcgacaattgagtgggccatgactgaactaacaaagaatccaaagataacagctaagcttagatcagaacttcaaacagtggtaggcgagagatcggtaaaagaatccgacttccccaatcttccataccttgaagctactgttaaagaaaccctaagacttcacccaccaactccattgctactcccacgtcgagcacttgaaacctgtacaatcctcaactacaccatcccaaaagattgtcaaattatggtcaacgcttggggaatcggacgtgatcccaagacttggatcgatccgttgactttctcaccagagagattcttgaattctagtgttgactttagggggaatgatttcagtttgataccatttggtgcaggaagaaggatatgccccggtctgccaatagcaaatcagtttattgcattgctagtggcaacatttgtgcaaaatttggattggtgtctaccaaatgggatgagtgttgaccatttgatagtggaggagaagtttgggttgactcttcaaaaagaaccacctctattcattgttcctaaatcaagggtttgatcttctcatcatggttcatgtgaaaactagattatctattgtgaaggctatgtatgttttctttccttcctataaaaaaaaaaaaaaaaaaaaaaaaaaaaaaaaaaaaa

SEQ ID NO: 65 - protein, 488 aa, Eschscholzia californica
  VARIANT 192: note = Xaa can be any naturally occurring amino acid
MEVVTVALIAVIISSILYLLFGSSGHKNLPPGPKPWPIVGNLLQLGEKPHAQFAELAQTYGDIFTLKMGTETVVVASTSSAASEILKTHDRILSARYVFQSFRVKGHVENSIVWSDCTETWKNLRKVCRTELFTQKMIESQAHVREKKCEEMVEYLMKKQGEEVKIVEVIFGTLVNIFGNLIFSQNIFELGXPNSGSSEFKEYLWRMLELGNSTNPADYFPMLGKFDLFGQRKEVAECLKGIYAIWGAMLQERKLAKKVDGYQSKNDFVDVCLDSGLNDYQINALLMELFGAGTETSASTIEWAMTELTKNPKITAKLRSELQTVVGERSVKESDFPNLPYLEATVKETLRLHPPTPLLLPRRALETCTILNYTIPKDCQIMVNAWGIGRDPKTWIDPLTFSPERFLNSSVDFRGNDFSLIPFGAGRRICPGLPIANQFIALLVATFVQNLDWCLPNGMSVDHLIVEEKFGLTLQKEPPLFIVPKSRV

SEQ ID NO: 66 - protein, 357 aa, Papaver somniferum
MGSLDAKPAAATQEVSIKDQAQLWNIIYGFADSLVLRCAVEIGIADIIKNNDGAITLAQLAAKLPITNVSSDYLYRMVRYLVHLNIIEQETCNGGVEKVYSLKPVGTLLLRDAERSMVPMILGMTQKDFMVSWHFMKEGLGNGSTTAFEKGMGMDIWKYLEGNPDQSQLFNEGMAGETRLLTKTLIEDCRDTFQGLDSLVDIGGGNGTTIKAIYEAFPHIKCTLYDLPHVVANSHDLPNIEKVPGDMFKSVPSAQAILLKLILHDWTDEECVNILKKCKEAIPKETGKVIIVDVALEEESNHELTKTRLILDIDMLVNTGGRERTADDWENLLKRAGFRSHKIRPIRAIQSVIEAFP

SEQ ID NO: 67 - DNA, 2664 bp, Papaver bracteatum
  source 1..2664: mol_type = other DNA
atggaattgcaatacttctcctacttccaacctacctcttctgttgttgctttgttgttggcattggtcagtatcttgttttccgttgttgttttgagaaagaccttctccaacaactattcttctccagcttcttctactgaaaccgctgttttgtgtcatcaaagacaacaatcttgcgccttgccaatttctggtttgttgcatgttttcatgaacaagaacggtttgatccatgttaccttgggtaatatggctgataagtacggtccaattttctcttttccaaccggttctcatagaaccttggttgtttcttcttgggaaatggtcaaagaatgtttcaccggtaacaacgataccttctttagtaacagaccaattccattggccttcaagattattttctatgccggtggtgttgactcttatggtttggctttggttccatacggtaaatattggagagaattgagaaagatctgcgtccacaacttgttgtccaatcaacaattattgaagttcagacacttgatcatctcccaagttgatacctccttcaacaagttatacgaattgtgcaagaactccgaagataatcaaggtatggttagaatggatgattggttggctcaattgtccttctcagttattggtagaatcgtttgcggtttccaatctgatccaaaaactggtgctccatctagagtcgaacaattcaaagaagctattaacgaagcctcctacttcatgtctacttctccagtttctgataacgttccaatgttgggttggatcgatcaattgactggtttgactagaaacatgacccattgtggtaagaagttggatttggttgtcgaatccatcatcaacgatcacagacaaaagagaagattctccagaacaaaaggtggtgacgaaaaggatgatgaacaagatgatttcatcgacatctgcttgtccattatggaacaaccacaattgccaggtaacaacaatccaccaaaaatcccaatcaagtccatcgttttggatatgattggtggtggtactgataccactaagttgactactatttggaccttgtccttgttgttgaacaacccacatgttttggacaaggctaaacaagaagttgacgctcatttcttgaccaagagaagaagtacaaacgacgctgctgttgttgatttcgatgacattagaaacttggtctacatccaagccattatcaaagaatccatgagattatacccagcctctccagttgttgaaagattgtctggtgaagattgtgttgttggtggttttcatgttccagctggtactagattgtgggttaatgtttggaagatgcaaagagatcctaacgtttgggctgatccaatggtttttagaccagaaagattcttgtcccacggtcaaaaaaagatggttgatgttagaggtaagaactacgaattattgccatttggtgccggtagaagaatttgtccaggtatttctttctccttggatttgatgcaattggtcttgaccagattgatcttggaattcgaaatgaagtctccatccggtaaggttgatatgactgctactccaggtttgatgtcttacaaagttgttccattggacatcttgttgacccatagaagaatcaagtcttgcgttcaattggcctcttctgaaagagatatggaatcttctggtgttccagttatcactttgagatctggtaaagttatgccagttttgggtatgggtacttttgaaaaagctggtaagggttccgaaagagaaagattggctattttgaaggccatcgaagttggttacagatactttgatactgctgctgcttacgaaaccgaagaagttttaggtgaagctattgctgaagccttgcaattgggtttaatcaagtcaagagatgaattattcatttcctccatgttgtggtgtactgatgctcatccagatagagttttgttggcattgcaaaactcattgagaaacttgaagttggaatacgtcgacttgtacatgttgccatttccagcttcattgaagccaggtaagattaccatggatatcccagaagaagatatctgcccaatggattatagatctgtttggtctgctatggaagaatgccaaaatttgggtttgaccaagtccattggtgtctctaatttctcctgcaaaaagttggaagaattgatggctactgctaacattccaccagctgtaaatcaagttgaaatgtctccagctttccaacaaaagaagttgagagaatactgcaacgctaacaacattttggtttccgccgtttctattttgggttctaatggtactccatggggttcaaatgctgttttaggttctgaagtcttgaaaaagattgctatggccaagggtaaatccgttgctcaagtttcaatgagatgggtttatgaacaaggtgcttccttggttgttaagtcctttagtgaagaaagattaagagaaaacttgaacatcttcgactggcaattgaccaaagaagataacgaaaagatcggtgaaatcccacaatgcagaattttgtctgcttacttcttggttagtccaaagggtccattcaagtctcaagaagaattatgggatgataaggcttaa

SEQ ID NO: 68 - protein, 887 aa, Papaver bracteatum
MELQYFSYFQPTSSVVALLLALVSILFSVVVLRKTFSNNYSSPASSTETAVLCHQRQQSCALPISGLLHVFMNKNGLIHVTLGNMADKYGPIFSFPTGSHRTLVVSSWEMVKECFTGNNDTFFSNRPIPLAFKIIFYAGGVDSYGLALVPYGKYWRELRKICVHNLLSNQQLLKFRHLIISQVDTSFNKLYELCKNSEDNQGMVRMDDWLAQLSFSVIGRIVCGFQSDPKTGAPSRVEQFKEAINEASYFMSTSPVSDNVPMLGWIDQLTGLTRNMTHCGKKLDLVVESIINDHRQKRRFSRTKGGDEKDDEQDDFIDICLSIMEQPQLPGNNNPPKIPIKSIVLDMIGGGTDTTKLTTIWTLSLLLNNPHVLDKAKQEVDAHFLTKRRSTNDAAVVDFDDIRNLVYIQAIIKESMRLYPASPVVERLSGEDCVVGGFHVPAGTRLWVNVWKMQRDPNVWADPMVFRPERFLSHGQKKMVDVRGKNYELLPFGAGRRICPGISFSLDLMQLVLTRLILEFEMKSPSGKVDMTATPGLMSYKVVPLDILLTHRRIKSCVQLASSERDMESSGVPVITLRSGKVMPVLGMGTFEKAGKGSERERLAILKAIEVGYRYFDTAAAYETEEVLGEAIAEALQLGLIKSRDELFISSMLWCTDAHPDRVLLALQNSLRNLKLEYVDLYMLPFPASLKPGKITMDIPEEDICPMDYRSVWSAMEECQNLGLTKSIGVSNFSCKKLEELMATANIPPAVNQVEMSPAFQQKKLREYCNANNILVSAVSILGSNGTPWGSNAVLGSEVLKKIAMAKGKSVAQVSMRWVYEQGASLVVKSFSEERLRENLNIFDWQLTKEDNEKIGEIPQCRILSAYFLVSPKGPFKSQEELWDDKA

SEQ ID NO: 69 - DNA, 1512 bp, Papaver bracteatum
  source 1..1512: mol_type = other DNA
atggccccaatcaacatcgaagaaaatgatttctggatgattgcctgcaccgttattatcgtttttgccttgatgaagttcatggtttccttctaccaatctgctaacactactgaatggccagaaggtccaaaaaccttgccaattattggtaacttgcatcaattgggtggtggtgttccattgcaagttgctttagctaatttggctaaagtttacggtggtgctttcaccatttggattggttcttgggttccaatgatcgttatctccgatattgataacgccagagaagttttggttaacaagtctgctgattactccgctagagatgttccagatattttgaagattattaccgccaacggtaagaacattgctgattgtgattctggtccattctggcatcatttgaagaagggtttacaatcctgcatcaacccatctaacgttatgtctttgtccagattgcaagaaaaggacatgcaaaacttgattaagtccatgcaagaaagagcctctcaacaaaacggtatcttgaaaccattggatcatgctaaagaagcctccatcagattattgtccagagttattttcggtcaagacttctccaacgaagatttggttattggtgttaaggatgccttggacgaaatggttagaatttctggtttggcttctttggctgatgctttcaaaattgctaagtacttgccatcccaaaaaaagaacatcagagatatgtacgccaccagagatagagtttacaacttgattcaaccacacatcgtcagtaatttgccagccaattcattcttgcatttcttgacctctcaagactactctgacgaaatcatctactccatggttttggaaattttcggtttgggtgttgattctactgctgctacagctgtttgggctttgtcttttttggttggtgaacaagaaatccaagaaaagttgtacagagaaatcaacaacttgaccggtggtcaaagaccagttaaggttgttgacttgaaagaattgccatacttgcaagccgttatgaaagaaaccttgagaatgaagccaattgctccattggctgttccacatgttgctgctaaagataccacttttaagggtagaagaatcgtcaagggtactaaggttatggttaacttgtacgccattcatcacgatccaaatgtttttccagctccatacaagttcatgcctgaaagatttttgaagggtgttaactccgatggtagatacggtgatattaacaccatggaatcctcattgattccatttggtgctggtatgagaatttgcggtggtgttgaattggcaaaacaaatggttggttttgctttggcctctatggtcaatgaatttaagtgggattgtgtctccgaaggtaacttaccagatttgtctgaagctatctccttcatcttgtacatgaagaatccattggaagctaaggttaccccaagaactaagccattcgattctagatga

SEQ ID NO: 70 - protein, 503 aa, Papaver bracteatum
MAPINIEENDFWMIACTVIIVFALMKFMVSFYQSANTTEWPEGPKTLPIIGNLHQLGGGVPLQVALANLAKVYGGAFTIWIGSWVPMIVISDIDNAREVLVNKSADYSARDVPDILKIITANGKNIADCDSGPFWHHLKKGLQSCINPSNVMSLSRLQEKDMQNLIKSMQERASQQNGILKPLDHAKEASIRLLSRVIFGQDFSNEDLVIGVKDALDEMVRISGLASLADAFKIAKYLPSQKKNIRDMYATRDRVYNLIQPHIVSNLPANSFLHFLTSQDYSDEIIYSMVLEIFGLGVDSTAATAVWALSFLVGEQEIQEKLYREINNLTGGQRPVKVVDLKELPYLQAVMKETLRMKPIAPLAVPHVAAKDTTFKGRRIVKGTKVMVNLYAIHHDPNVFPAPYKFMPERFLKGVNSDGRYGDINTMESSLIPFGAGMRICGGVELAKQMVGFALASMVNEFKWDCVSEGNLPDLSEAISFILYMKNPLEAKVTPRTKPFDSR

SEQ ID NO: 71 - protein, 311 aa, Papaver bracteatum
MPETCPNTVTKMRCAVVTGGNKGIGFEICKQLSSSGIMVVLTCRDVTRGLEAVEKLKNSNHENVVFHQLDVTDPITTMSSLADFIKARFGKLDILVNNAGVAGFSVDADRFKAMISDIGEDSEEVVKIYEKPEAQELMSETYELAEECLKINYYGVKSVTEVLLPLLQLSDSPRIVNVSSSTGSLKYVSNETALEILGDGDALTEERIDMVVNMLLKDFKENLIETNGWPSFGAAYTTSKACLNAYTRVLAKKIPKFQVNCVCPGLVKTEMNYGIGNYTADEGAKHVVRIALFPDDGPSGFFYDCSELSAF

SEQ ID NO: 72 - skipped

SEQ ID NO: 73 - protein, 474 aa, Papaver somniferum
MATMYSAAVEVISKETIKPTTPTPSQLKNFNLSLLDQCFPLYYYVPIILFYPATAANSTGSSNHHDDLDLLKSSLSKTLVHFYPMAGRMIDNILVDCHDQGINFYKVKIRGKMCEFMSQPDVPLSQLLPSEVVSASVPKEALVIVQVNMFDCGGTAICSSVSHKIADAATMSTFIRSWASTTKTSRSGGSTAAVTDQKLIPSFDSASLFPPSERLTSPSGMSEIPFSSTPEDTEDDKTVSKRFVFDFAKITSVREKLQVLMHDNYKSRRQTRVEVVTSLIWKSVMKSTPAGFLPVVHHAVNLRKKMDPPLQDVSFGNLSVTVSAFLPATTTTTTNAVNKTINSTSSESQVVLHELHDFIAQMRSEIDKVKGDKGSLEKVIQNFASGHDASIKKINDVEVINFWISSWCRMGLYEIDFGWGKPIWVTVDPNIKPNKNCFFMNDTKCGEGIEVWASFLEDDMAKFELHLSEILELI

SEQ ID NO: 74 - DNA, 537 bp, Coptis japonica
  source 1..537: mol_type = other DNA
atggaaagattgatcttcaatggtagacctttgttgcacagagttaccaaagaagaaaccgttatgttgtaccacgaattggaagttgctgcttctgctgatgaagtttggtctgttgaaggttctccagaattgggtttacatttgccagatttgttgccagctggtatttttgccaagttcgaaattactggtgatggtggtgaaggttccattttggatatgacttttccaccaggtcaattcccacatcattacagagaaaagttcgtctttttcgaccacaagaacagatacaagttggtcgaacaaatcgatggtgatttcttcgatttgggtgttacttactacatggacaccattagagttgttgctactggtccagattcttgcgttattaagtctactactgaataccacgtcaagccagaatttgctaaaatcgttaagccattgatcgataccgttccattggctattatgtctgaagctattgccaaggttgtcttggaaaacaaacacaagtcatctgaatga

SEQ ID NO: 75 - protein, 178 aa, Coptis Japonica
MERLIFNGRPLLHRVTKEETVMLYHELEVAASADEVWSVEGSPELGLHLPDLLPAGIFAKFEITGDGGEGSILDMTFPPGQFPHHYREKFVFFDHKNRYKLVEQIDGDFFDLGVTYYMDTIRVVATGPDSCVIKSTTEYHVKPEFAKIVKPLIDTVPLAIMSEAIAKVVLENKHKSSE

SEQ ID NO: 76 - protein, 705 aa, Beta vulgaris
MDSNSIKVSPLDLMSALLKGNFEQLNASFEDSSAAALIVENREVLMFVTTSIAVLIGCAFLFIWKKSNGGKSSKVVSELPKPLILKDDDVEVDDGKKKVTVFFGTQTGTAEGFAKALVEEAKARYEKATFKIVDLDDYAGDDDEYERKLKKENLAFFFLATYGDGEPTDNAARFYKWFLEGKDRGEWLQNLEYGVFGLGNRQYEHFNKVGKEIDELLAEQGAKRLVPVGLGDDDQCIEDDFTAWKDLVWPELDRLLRDEDDVAVSTPYTATIPEYRVVFHEAAEVAQQDKSWANANGHAVFDAQHPCRSNVAVRRELHTPASDRSCTHLEFDVSGTGLKYETGDHVGVYCENLVETVEEAERLLGLKDTYFSIHTDNEDGTPVAGTSLPPPFPPCTLRSALTRYADLLSSPKKSALLALAAHASDPNEAKRLKYLASPAGKDEYSQWVVASQRSLLEVMAEFPSAKPPLGVFFAAVAPRLQPRYYSISSSPRVAPSRIHVTCALVLDKMPTGRIHKGVCSTWMKNSVAQGESHDCSWAPIFVRQSNFKLPADSSVPIIMIGPGTGLAPFRGFLQERSALKEDGVDLGQAILFFGCRNRKVDFIYEDELQNFVETGVLSELVVAFSREGPTKEYVQHKMMERASDVWNVINQGGYVYVCGDAKGMARDVHRVLHTILQEQGGMGSSQAEGMVKNLQTTGRYLRDVW

SEQ ID NO: 77 - protein, 256 aa, Saccharomyces cerevisiae
MDFTKPETVLNLQNIRDELVRMEDSIIFKFIERSHFATCPSVYEANHPGLEIPNFKGSFLDWALSNLEIAHSRIRRFESPDETPFFPDKIQKSFLPSINYPQILAPYAPEVNYNDKIKKVYIEKIIPLISKRDGDDKNNFSSVATRDIECLQSLSRRIHFGKFVAEAKFQSDIPLYTKLIKSKDVEGIMKNITNSAVEEKILERLTKKAEVYGVDPTNESGERRITPEYLVKIYKEIVIPITKEVEVEYLLRRLEE

SEQ ID NO: 78 - DNA, 2118 bp, Beta vulgaris
  source 1..2118: mol_type = other DNA
atggactccaactccatcaaggtttctccattggatttgatgtctgctttgttgaagggtaacttcgaacaattgaacgcctctttcgaagattcttctgctgctgctttgatcgttgaaaacagagaagttttgatgttcgtcaccacctctattgctgttttgattggttgtgccttcttgttcatctggaaaaagtctaacggtggcaagtcctctaaagttgtttctgaattgccaaagccactgatcttgaaggatgatgatgttgaagttgatgacggtaagaagaaggttactgttttcttcggtactcaaactggtactgctgaaggttttgctaaagctttggttgaagaggctaaagccagatacgaaaaggctacttttaagatcgttgacttggatgattacgccggtgatgatgacgaatacgaaagaaagttgaagaaagagaacctggctttcttcttcttggctacttatggtgatggtgaacctactgataatgctgctagattttacaagtggttcttggaaggtaaggatagaggtgaatggttgcaaaatttggagtacggtgtttttggtttgggtaacagacaatacgaacacttcaacaaggtcggtaaagaaatcgatgaactgttggctgaacaaggtgctaaaagattggttccagttggtttaggtgatgacgatcaatgtatcgaagatgattttaccgcttggaaagatttggtatggccagaattggacagactgttgagagatgaagatgatgtagctgtttctactccatacactgctactattccagagtacagagttgtttttcatgaagctgctgaagttgcccaacaagataagtcttgggctaatgctaatggtcatgctgtttttgatgctcaacatccatgtagatctaacgttgctgttagaagagaattgcatactccagcttcagatagatcttgtacccatttggaattcgatgtttctggtactggtttgaagtacgaaactggtgatcatgttggtgtttactgcgaaaacttggttgaaactgttgaagaagctgaaaggttgttgggtttgaaggatacctacttctctatccataccgataacgaagatggtactccagttgctggtacttctttgccaccaccatttccaccatgtactttgagatctgctttgactagatacgccgatttgttgtcctctccaaaaaaatctgctttattggccttggctgctcatgcttctgatccaaatgaagctaagagattgaagtacttggcttctccagctggtaaagatgaatattctcaatgggttgttgcctctcagagatctttgttggaagttatggctgaatttccatctgctaaaccaccattgggtgttttttttgctgctgttgctccaagattgcagccaagatattactccatttcatcttctccaagagttgccccatctagaattcatgttacttgtgctttggtgttggataagatgccaactggtagaattcacaagggtgtttgttcaacctggatgaagaattctgttgctcaaggtgaatctcatgattgctcttgggctccaattttcgttagacaatctaactttaagttgcccgctgattcttccgttccaattatcatgattggtccaggtacaggtttggctccttttagaggttttctacaagaaaggtccgccttgaaagaagatggcgttgatttgggtcaagccattttgttttttggctgcagaaacagaaaggtcgacttcatatacgaagatgagttgcaaaacttcgtcgaaacaggtgttttgtccgaattggttgttgctttttctagagaaggtcccaccaaagaatacgttcaacataagatgatggaaagagcttctgacgtttggaacgttatcaatcaaggtggttacgtttacgtttgcggtgatgctaaaggtatggcaagagatgttcatagagtcttgcataccatcctacaagagcaaggtggtatgggttcttctcaagctgaaggtatggttaagaacttgcaaactactggtagatacttgagggatgtttggtaa

SEQ ID NO: 79 - protein, 180 aa, Papaver somniferum
MDSINSSIYFCAYFRELIIKLLMAPLGVSGLVGKLSTELEVDCDAEKYYNMYKHGEDVKKAVPHLCVDVKIISGDPTSSGCIKEWNVNIDGKTIRSVEETTHDDETKTLRHRVFEGDVMKDFKKFDTIMVVNPKPDGNGCVVTRSIEYEKTNENSPTPFDYLQFGHQAIEDMNKYLRDSE

SEQ ID NO: 80 - protein, 160 aa, Papaver somniferum
MAPLGVSGLVGKLSTELEVDCDAEKYYNMYKHGEDVKKAVPHLCVDVKIISGDPTSSGCIKEWNVNIDGKTIRSVEETTHDDETKTLRHRVFEGDVMKDFKKFDTIMVVNPKPDGNGCVVTRSIEYEKTNENSPTPFDYLQFGHQAIEDMNKYLRDSESN